(19) United States
(12) Patent Application Publication
(10) Pub. No.: US 2025/0352666 A1
(43) Pub. Date: Nov. 20, 2025

(54) LIPID NANOPARTICLE COMPOSITIONS AND USES THEREOF

(71) Applicant: Generation Bio Co., Cambridge, MA (US)

(72) Inventors: Phillip Samayoa, Cambridge, MA (US); Nathaniel Silver, Cambridge, MA (US); Gregory Feinstein, Cambridge, MA (US); Michelle LeBlanc, Cambridge, MA (US)

(21) Appl. No.: 18/871,840

(22) PCT Filed: Jun. 7, 2023
(86) PCT No.: PCT/US2023/024642

§ 371 Date: Dec. 5, 2024

Related U.S. Application Data
(60) Provisional application No. 63/349,712, filed on Jun. 7, 2022.

(51) Int. Cl. A61K 48/00; A61K 9/00; A61K 9/51

(52) U.S. Cl. CPC A61K 48/0033; A61K 9/0048; A61K 9/5123; A61K 48/0058; A61K 48/0075; C12N 2750/14143

(57) ABSTRACT

The present disclosure relates to pharmaceutical compositions comprising a lipid nanoparticle (LNP), a therapeutic nucleic acid (TNA) and at least one pharmaceutically acceptable excipient, wherein the LNP comprises at least one lipid, and wherein the LNP is capable of delivering the TNA to a retinal cell. Methods of treating ocular diseases and disorders are also provided.

### RELATED APPLICATIONS

This application claims priority to U.S. Provisional Application No. 63/349,712, filed on Jun. 7, 2022, the contents of which are hereby incorporated by reference in their entirety.

### BACKGROUND

Vision disabilities are one of the top ten disabilities in humans. According to the Centers for Disease Control and Prevention, more than 3 million people in the United States have vision impairment. By 2050, this number is expected to grow to ˜6.95 million people (see CDC Publication “The Burden of Vision Loss”; accessed on May 13, 2022). Inherited retinal degenerations (IRDs) represent a diverse group of progressive, visually debilitating diseases that can lead to blindness. The prevalence of monogenic IRDs is approximately 1 in 2000 individuals (Berger W. et al., Prog. Retin. Eye. Res. 29, 335-375 (2010)).

The retina is a light-sensitive tissue at the back of the eye that is made up of a variety of cell types, including photoreceptor cells, retinal pigmented epithelial (RPE) cells, and retinal ganglion cells. There are over 300 genes associated with IRDs, which lead to the dysfunction and degeneration of photoreceptors and/or the RPE (Daiger, S. P. et al., The Retinal Information Network; The University of Texas Health Science Center at Houston: Houston, TX, USA, 1996). Gene therapy is a promising technology for treating both inherited and acquired retinal diseases through gene replacement.

Adeno-associated virus (AAV) vectors have been the viral vector of choice for retinal gene transfer. In 2017, a gene therapy called LUXTURNA® was approved by the U.S. Food and Drug Administration for use in children and adults with retinal disease due to two mutations in the gene RPE65. In this therapy, the RPE65 gene is packaged into an AAV vector, and injected into the eye. In clinical trials, the genetically modified AAV was able to significantly restore vision in blind children, enabling them to complete tasks such as walking through a maze without bumping into soft objects, or catching a ball. Although an effective technique has been established for delivering a gene into the retina, challenges remain. Some genes, such as the 6.8 kb ABCA4 gene causing Stargardt's hereditary maculopathy, an inherited disorder of the macula that typically causes vision loss during childhood or adolescence, are too large to be carried into the retina by the AAV virus, so other approaches are needed. What remains clear is that the choice of vector and the mode of administration are important determinants of the efficacy and safety of ocular therapeutics.

Ionizable lipid nanoparticles (LNPs) have been widely used for the systemic delivery of gene therapeutics, e.g., RNA therapeutics. Various types of ionizable lipid materials have been previously reported for LNP formulations, such as C12-200, cKK-E12, and DLin-MC3-DMA, and efficient gene silencing in the liver at a dosing level of 0.002 mg of siRNA/kg has been demonstrated (Dong, et al., Proc. Natl. Acad. Sci. U.S.A. 111, 3955-3960 (2014)). Although the inclusion of targeting ligands has been shown to enhance the delivery and therapeutic efficiency of mRNA-LNPs, it has been recognized that attaching targeting moieties may add complexity, cost, and regulatory difficulties to the process of manufacturing LNP systems (Cheng et al., Science. 2012 Nov. 16; 338(6109):903-10). In addition, it has been demonstrated that the targeting specificity of some targeting ligands may disappear when lipid nanoparticles are exposed to biological fluids where interaction with proteins in the media and the consequent formation of protein corona takes place (Salvati et al., Nat Nanotechnol. 2013 February; 8(2):137-43).

However, to fully realize the potential of nucleic acid therapeutics for ocular diseases and disorders, a targeted, efficient and long-lasting in vivo delivery system is needed.

### SUMMARY

AAV vectors are currently the viral vector of choice for retinal gene transfer. However, the optimal method to deliver these treatments to the photoreceptor (PR) cells remains to be improved, with a goal to increase transduction efficacy and to reduce complications associated with the highly invasive surgery required for subretinal injection of the viral vector suspension. Previously, when LNPs have been used for retinal gene transduction, the majority of the expression has been seen in the retinal pigmented epithelium (RPE) (Patel et al., Journal of Controlled Release Volume 303, 10 Jun. 2019, Pages 91-100), while getting into the other layers of the retina where the photoreceptor cells are, has remained a challenge.

The present disclosure describes for the first time the combination of a LNP incorporating, for example, Lipid A or Lipid 58 as the ionizable lipid, with an mRNA cargo for retinal delivery. Using mouse, rat, and non-human primate (NHP) in vivo systems, the present disclosure surprisingly demonstrated that GFP expression can be achieved evenly in the RPE cells (eye cup) and PR cells (neural retina) using LNP/GFP mRNA constructs. Importantly, the data presented herein shows that saturation can be achieved at low doses of LNP/mRNA construct; that is, the dose of LNPs does not need to be high for expression, which is an important consideration for toxicity. The results presented in the instant disclosure surprisingly demonstrate not only are the LNP compositions of the invention consistently able to be internalized by the photoreceptor cells, but they are able to do so and achieve satisfactory expression at low doses and without causing toxicity issues such as retinal degradation. In one aspect, the disclosure provides a pharmaceutical composition comprising a lipid nanoparticle (LNP), a therapeutic nucleic acid (TNA) and at least one pharmaceutically acceptable excipient, wherein the LNP comprises at least one lipid, and wherein the LNP is capable of delivering the TNA to a retinal cell. According to some embodiments, the LNP is capable of delivering the TNA to a photoreceptor (PR) cell. According to some embodiments, the LNP is capable of delivering the TNA to a retinal pigment epithelium (RPE) cell. According to some embodiments of the aspects and embodiments herein, the LNP is capable of delivering the TNA to a photoreceptor (PR) cell and a retinal pigment epithelium (RPE) cell, wherein expression of the TNA in the PR cell and expression of the TNA in RPE cell is evenly distributed. According to some embodiments of the aspects and embodiments herein, the pharmaceutical composition is for administration to a subject. According to some embodiments, the pharmaceutical composition is for administration to a subject via subretinal injection, suprachoroidal injection, or intravitreal injection. According to some embodiments, the LNP/TNA is for administration at a dose of about 0.03 μg to about 2.0 μg, for example about 0.03 μg to about 1.5 μg, about 0.05 μg to about 2.0 μg, about 1.0 μg to about 1.5 μg, about 1.0 μg to about 2.0 μg, about 1.5 μg to about 2.0 μg, about 0.5 μg to about 1.0 μg, or about 0.5 μg to about 1.5 μg. According to some embodiments, the LNP/TNA is for administration at a dose of about 0.1 μg to about 1.0 μg, for example about 0.1 μg to about 0.5 μg or about 0.5 μg to about 1.0 μg or about 0.25 μg to about 0.5 μg or about 0.1 μg to about 0.25 μg or about 0.75 μg to about 1.0 μg. According to some embodiments, the LNP/TNA is for administration at a dose of about 0.1 μg to about 0.5 μg, for example 0.1 μg, 0.2 μg, 0.25 μg, 0.3 μg, 0.35 μg, 0.4 μg, 0.45 μg or 0.5 μg. According to some embodiments, the subject is a human in need of treatment with LNP encapsulated with TNA. According to some embodiments of the aspects and embodiments herein, the LNP is capable of being internalized into the PR cell and/or the RPE cell. According to some embodiments of the aspects and embodiments herein, retinal degeneration does not exceed more than 5% of outer nuclear layer (ONL) loss after 7 days. According to some embodiments of the aspects and embodiments herein, retinal degeneration does not exceed more than 5% of outer nuclear layer (ONL) loss after 14 days. According to some embodiments of the aspects and embodiments herein, retinal degeneration does not exceed more than 5% of outer nuclear layer (ONL) loss after 21 days. According to some embodiments of the aspects and embodiments herein, retinal degeneration does not exceed more than 5% of outer nuclear layer (ONL) loss after 4 weeks. According to some embodiments of the aspects and embodiments herein, retinal degeneration does not exceed more than 10% of outer nuclear layer (ONL) loss after 7 days. According to some embodiments of the aspects and embodiments herein, retinal degeneration does not exceed more than 10% of outer nuclear layer (ONL) loss after 14 days. According to some embodiments of the aspects and embodiments herein, retinal degeneration does not exceed more than 10% of outer nuclear layer (ONL) loss after 21 days. According to some embodiments of the aspects and embodiments herein, retinal degeneration does not exceed more than 10% of outer nuclear layer (ONL) loss after 4 weeks. According to some embodiments of the aspects and embodiments herein, retinal degeneration does not exceed more than 15% of outer nuclear layer (ONL) loss after 7 days. According to some embodiments of the aspects and embodiments herein, retinal degeneration does not exceed more than 15% of outer nuclear layer (ONL) loss after 14 days. According to some embodiments of the aspects and embodiments herein, retinal degeneration does not exceed more than 15% of outer nuclear layer (ONL) loss after 21 days. According to some embodiments of the aspects and embodiments herein, retinal degeneration does not exceed more than 15% of outer nuclear layer (ONL) loss after 4 weeks. According to some embodiments of the aspects and embodiments herein, ONL loss is measured by Optical Coherence Tomography (OCT). According to some embodiments of the aspects and embodiments herein, the LNP comprises a lipid selected from the group consisting of: a cationic lipid, a sterol or a derivative thereof, a non-cationic lipid, and a PEGylated lipid. According to some embodiments of the aspects and embodiments herein, the TNA is encapsulated in the lipid. According to some embodiments of the aspects and embodiments herein, the TNA is selected from the group consisting of minigenes, plasmids, minicircles, small interfering RNA (siRNA), microRNA (miRNA), antisense oligonucleotides (ASO), ribozymes, closed-ended (ceDNA), ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, gRNA, DNA viral vectors, viral RNA vector, non-viral vector and any combination thereof. According to some embodiments, the TNA is ceDNA. According to some embodiments, the ceDNA is linear duplex DNA. According to some embodiments, the TNA is mRNA. According to some embodiments, the TNA is siRNA. According to some embodiments, the TNA is a plasmid.

According to some embodiments of the aspects and embodiments herein, the pharmaceutical composition is administered to a subject. According to some embodiments, the subject is a human patient in need of treatment with the TNA encapsulated by the LNP.

According to some embodiments, the cationic lipid is represented by Formula (I):

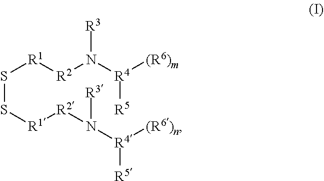

or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl; or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or -[C(R^{a})₂]₂CR^{a};
R^{a}, for each occurrence, is independently H or C₁₋₃ alkyl;
or alternatively, when R⁴ is —C(R^{a})₂CR^{a}, or -[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or -[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

According to some embodiments, the cationic lipid is represented by Formula (II):

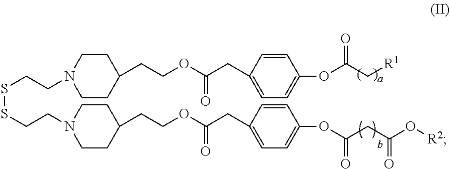

or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

According to some embodiments, the cationic lipid is 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(tridecan-5-yl) nonanedioate (Lipid 58), represented by the following structural formula:

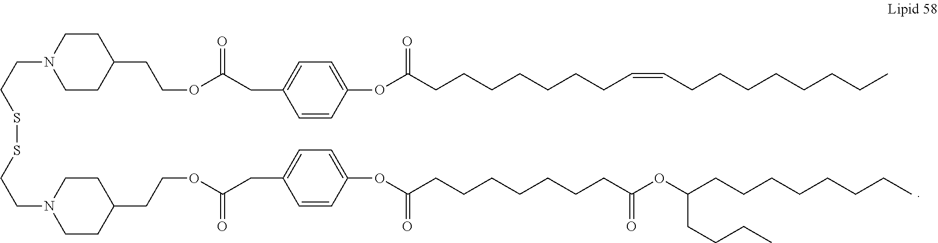 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

According to some embodiments, the lipid is represented by the Formula (V):

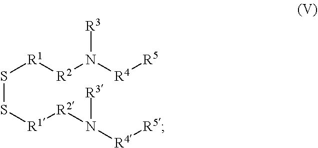

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

According to some embodiments, the cationic lipid is represented by Formula (XV):

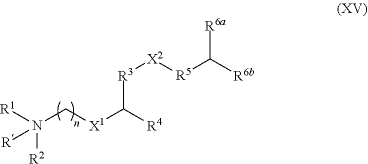

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl; R³ is C₁-C₁₂ alkylene or C₂-C₁₂ alkenylene;
R⁴ is C₁-C₁₆ unbranched alkyl, C₂-C₁₆ unbranched alkenyl, or

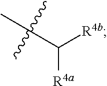

wherein:
R^{4a} and R^{4b} are each independently C₁-C₁₆ unbranched alkyl or C₂-C₁₆ unbranched alkenyl;
R⁵ is absent, C₁-C₅ alkylene, or C₂-C₅ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and n is an integer selected from 1, 2, 3, 4, 5, and 6.

According to some embodiments, the cationic lipid is represented by Formula (XX):

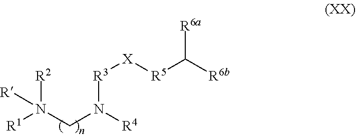

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R′, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or

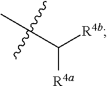

wherein:
R^{4a} and R^{4b} are each independently C₁-C₆ unbranched alkyl or C₂-C₆ unbranched alkenyl;
R⁵ is absent, C₁-C₆ alkylene, or C₂-C₆ alkenylene;
R^{6a} and R^{4b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6. According to some embodiments, the cationic lipid is selected from any lipid in Table 2, Table 5, Table 6, Table 7, or Table 8.

According to some embodiments, the cationic lipid is Lipid A represented by the following structure:

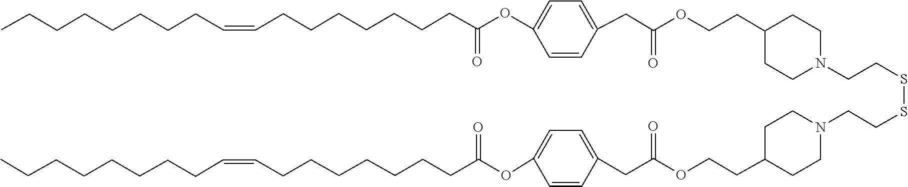

or a pharmaceutically acceptable salt thereof.

According to some embodiments, the cationic lipid is 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(tridecan-5-yl) nonanedioate (Lipid 58), represented by the following structural formula:

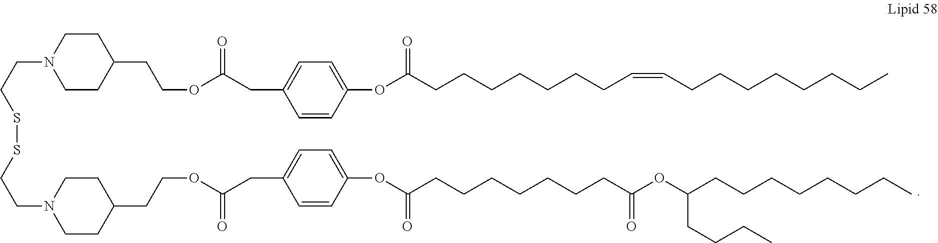 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

or a pharmaceutically acceptable salt thereof.

According to some embodiments, the sterol or a derivative thereof is a cholesterol.

According to some embodiments, the sterol or a derivative thereof is beta-sitosterol.

According to some embodiments, the non-cationic lipid is selected from the group consisting of distearoyl-sn-glycero-phosphoethanolamine (DSPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, lysophosphatidylcholine, dilinoleoylphosphatidylcholine, and mixtures thereof. According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE).

According to some embodiments, the PEGylated lipid is selected from the group consisting of PEG-dilauryloxypropyl; PEG-dimyristyloxypropyl; PEG-dipalmityloxypropyl, PEG-distearyloxypropyl; l-(monomethoxy-polyethyleneglycol)-2,3-dimyristoylglycerol (DMG-PEG); 1,2-distearoyl-sn-glycero-3-phosphoethanolamine-N-[azido(polyethylene glycol], and distearoyl-rac-glycerol-poly(ethylene glycol) (DSG-PEG); PEG-dilaurylglycerol; PEG-dipalmitoylglycerol; PEG-disterylglycerol; PEG-dilaurylglycamide; PEG-dimyristylglycamide; PEG-dipalmitoylglycamide; PEG-disterylglycamide; (1-[8′-(Cholest-5-en-3[beta]-oxy)carboxamido-3′,6′-dioxaoctanyl]carbamoyl-[omega]-methyl-poly(ethylene glycol) (PEG-cholesterol); 3,4-ditetradecoxylbenzyl-[omega]-methyl-poly(ethylene glycol) ether (PEG-DMB), and 1,2-dimyristoyl-sn-glycero-3-phosphoethanolamine-N-[methoxy(polyethylene glycol) (DSPE-PEG), and 1,2-distearoyl-sn-glycero-3-phosphoethanolamine-N-poly(ethylene glycol)-hydroxyl (DSPE-PEG-OH). According to some embodiments, the PEGylated lipid is DMG-PEG, DSPE-PEG, DSPE-PEG-OH, DSG-PEG, or a combination thereof. According to some embodiments, the PEGylated lipid is DMG-PEG2000, DSPE-PEG2000, DSPE-PEG2000-OH, DSG-PEG2000, or a combination thereof.

According to some embodiments of the aspects and embodiments herein, the cationic lipid is present at a molar percentage of about 30% to about 80%, for example about 30% to about 70%, about 30% to about 60%, about 30% to about 50%, about 30% to about 40%, about 40% to about 80%, about 40% to about 70%, about 40% to about 60%, about 40% to about 50%, about 50% to about 80%, about 50% to about 70%, about 50% to about 60%, about 60% to about 80%, about 60% to about 70%, or about 70% to about 80%. According to some embodiments of the aspects and embodiments herein, the sterol is present at a molar percentage of about 20% to about 50%, about 20% to about 40%, about 20% to about 30%, about 30% to about 50%, about 30% to about 40%, or about 40% to about 50%. According to some embodiments of the aspects and embodiments herein, the non-cationic lipid is present at a molar percentage of about 2% to about 20%, for example about 2% to about 15%, about 2% to about 10%, about 2% to about 5%, about 5% to about 20%, about 5% to about 15%, about 5% to about 10%, about 10% to about 15%, about 10% to about 20%, or about 15% to about 20%. According to some embodiments of the aspects and embodiments herein, the PEGylated lipid is present at a molar percentage of about 2.1% to about 10%, for example about 2.1% to about 5% or about 5% to about 10% or wherein the PEGylated lipid is present at a molar percentage of about 1% to about 2%, for example about 1%, 1.1%, 1.2%, 1.3%, 1.4%, 1.5%, 1.6%, 1.7%, 1.8%, 1.9% or 2% According to some embodiments of the aspects and embodiments herein, the pharmaceutical composition further comprises dexamethasone palmitate. According to some embodiments, the LNP comprises Lipid A, DOPC, cholesterol and DMG-PEG. According to some embodiments, the LNP comprises Lipid A, DOPC, cholesterol, DMG-PEG, and DSPE-PEG. According to some embodiments, the LNP comprises Lipid A, DOPE, cholesterol and DMG-PEG. According to some embodiments, the LNP comprises Lipid A, DOPE, cholesterol, DMG-PEG, and DSPE-PEG. According to some embodiments, the LNP comprises Lipid A, DSPC, cholesterol and DMG-PEG. According to some embodiments, the LNP comprises Lipid A, DSPC, cholesterol, DMG-PEG, and DSPE-PEG. According to some embodiments, the LNP comprises Lipid A, DOPC, beta-sitosterol and DMG-PEG. According to some embodiments, the LNP comprises Lipid A, DOPC, beta-sitosterol, DMG-PEG, and DSPE-PEG. According to some embodiments, the LNP comprises Lipid A, DOPE, beta-sitosterol and DMG-PEG. According to some embodiments, the LNP comprises Lipid A, DOPE, beta-sitosterol, DMG-PEG, and DSPE-PEG. According to some embodiments, the LNP comprises Lipid A, DSPC, beta-sitosterol and DMG-PEG. According to some embodiments, the LNP comprises Lipid A, DSPC, beta-sitosterol, DMG-PEG, and DSPE-PEG.

According to some embodiments of the aspects and embodiments herein, the DMG-PEG is DMG-PEG2000. According to some embodiments of the aspects and embodiments herein, the DSPE-PEG is DSPE-PEG2000. According to some embodiments of the aspects and embodiments herein, the e DSPE-PEG is DSPE-PEG5000. According to some embodiments, the LNP comprises Lipid A, DOPC, sterol, DMG-PEG and DSPE-PEG at molar ratios of about 51:7.3:38.3:2.9:0.5. According to some embodiments of the aspects and embodiments herein, the LNP has a total lipid to TNA ratio of about 10:1 to about 40:1, for example about 10:1, about 15:1, about 20:1, about 25:1 about 30:1, about 35:1, or about 40:1.

According to other aspects, the disclosure provides a method of treating an ocular disorder in a subject, the method comprising administering to the subject a therapeutically effective amount of the pharmaceutical composition of any one of the aspects and embodiments herein. According to some embodiments, the subject is a human.

According to some embodiments of the aspects and embodiments herein, the ocular disorder is Stargardt macular dystrophy. According to some embodiments of the aspects and embodiments herein, the ocular disorder is LCA10. According to some embodiments of the aspects and embodiments herein, the ocular disorder is Usher syndrome. According to some embodiments of the aspects and embodiments herein, the ocular disorder is wet AMD.

According to some aspects, the disclosure provides a method of treating a genetic disorder in a subject, comprising administering to the subject an effective amount of the pharmaceutical composition of any one of the aspects and embodiments herein. According to some embodiments, the subject is a human. According to some embodiments, the genetic disorder is an ocular disorder.

According to some aspects, the disclosure provides a method of delivering a therapeutic nucleic acid (TNA) or increasing the concentration of the TNA in the retina of a subject, the method comprising administering to the subject a therapeutically effective amount of the pharmaceutical composition of any one of the aspects and embodiments herein.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A-1D present graphs that show LNP-delivered mRNA and LNP-delivered ceDNA were tolerated in both mouse (bottom panels, FIG. 1B and FIG. 1D) and rat (top panels, FIG. 1A and

FIG. 1C). Degeneration score is shown at Day 21.

FIGS. 2A-2I show the results of fundus imaging for GFP expression in rats and mice at 24 hours, as described in Example 2. FIGS. 2A-2I show fundus imaging of Lipid A LNP/GFP mRNA treated mice (0.4 μg) (Group 2 as described in Table 9, FIGS. 2A-2E) compared to non-treated control mice (Group 1 as described in Table 9, FIGS. 2F-2I).

FIG. 3 is a graph that shows the amount of GFP in the neural retina and RPE/eye cup as determined by ELISA after dosing of wild type mice with Lipid A LNP/GFP mRNA (0.4 μg) (Group 2 as described in Table 9) at 12 hours and 24 hours post-treatment.

FIG. 4A and FIG. 4B show a comparison of GFP expression pattern in Lipid A LNP/GFP mRNA (0.4 μg) (FIG. 4B) compared to GFP transgenic mice (FIG. 4A).

FIG. 5A and FIG. 5B show a comparison of GFP expression pattern in Lipid A LNP/GFP mRNA (0.4 μg) (FIG. 5B) compared to non-treated vehicle control mice (FIG. 5A).

FIG. 6 is a graph quantifying GFP expression. FIG. 6 shows that Lipid A LNP/GFP mRNA delivery in mice resulted in an even distribution of GFP expression within the neural retina and eye cup.

FIGS. 7A-7E show GFP expression in the neural retina and RPE in mice treated with Lipid A LNP/GFP mRNA (0.4 μg) and AAV.GFP (AAV5-CAG-GFP), as described in Example 2. FIG. 7A-FIG. 7D are images showing the results from immunohistochemistry (IHC). FIG. 7E is a graph quantifying the results.

FIGS. 8A and 8B are graphs that quantitate GFP expression by ELISA in the neural retina (with photoreceptors or PR) and eyecup (with retinal pigment epithelium or RPE cells) at increasing doses (0.2 μg, 0.4 μg, 1.0 μg) at 12 and 24 hours, with the GFP concentration expressed as ng/eye (FIG. 8A) and ng/μg cargo (FIG. 8B).

FIGS. 9A-9F show the results of fundus imaging in mouse and rat models as described in Example 4. When LNP-delivered mRNA such as Lipid A LNP/GFP mRNA was dose matched in the mouse and rat models, GFP expression by fundus in the rat was found to be comparable to that in the mouse. As shown in FIG. 9E and FIG. 9F, Lipid A LNP/GFP mRNA given at the medium and high doses to rats (0.3 μg and 1.2 μg, respectively) achieved expression levels in rats that were comparable to the expression levels of Lipid A LNP/GFP mRNA given at the medium and high doses to mice (0.1 μg and 0.4 μg, respectively, see FIG. 9B and FIG. 9C)

FIGS. 10A-10D are images that show retinal degeneration in mice treated as described in Example 4. FIG. 10A shows vehicle treatment for reference. The images in FIGS. 10B, 10C. and 10D show that no retinal degeneration occurred at day 1 after the mice were administered with increased Lipid A LNP/GFP mRNA doses of 0.03 μg, 0.1 μg, and 0.4 μg, thereby indicating a large tolerability window for LNP-delivered mRNA.

FIGS. 11A-11E show color fundus imaging of mouse eyes at day 2 post-treatment via subretinal injections with various LNP compositions formulated with GFP mRNA and with different ionizable lipids as described in Table 12 (all 0.2 μg dose) while FIGS. 11F-11J show corresponding cobalt blue fundus imaging (for GFP expression) of the same mouse eye samples.

FIG. 12 is a graph that quantifies GFP expression in both the neural retina and eye cup from the experiments performed in Example 5.

FIG. 13A and FIG. 13B are graphs showing day 1 inflammation scores and degeneration scores, respectively, of mice eyes post-treatment via subretinal injections with various LNP compositions formulated with GFP mRNA and with different ionizable lipids as described in this example (all 0.2 μg dose). FIG. 13C and FIG. 13D are graphs showing day 1 inflammation scores and degeneration scores, respectively, of the same samples.

FIGS. 14A-14F are panels showing the results of OCT imaging at day 1 as described in Example 5. FIG. 14A shows the vehicle reference. FIG. 14B shows Lipid A LNP/GFP mRNA, FIG. 14C shows MC3 LNP/GFP mRNA, FIG. 14D and FIG. 14E show control (CTRL) Lipid Z LNP 1/GFP mRNA and control (CTRL) Lipid Z LNP 2/GFP mRNA, respectively, and FIG. 14F shows Lipid 58 LNP/GFP mRNA.

FIGS. 15A-15F are panels showing the results of OCT imaging at day 28 as described in Example 5. FIG. 15A shows the vehicle reference. At day 28, degeneration scores as high as ˜2.0 were observed in LNP compositions formulated with either MC3 or control (CTRL) Lipid Z; and such high degeneration scores were corroborated by the thinning of the outer nuclear layer (ONL) or retinal degeneration seen in OCT images taken on day 28 (see FIGS. 15C-15E). In contrast, at day 28, Lipid A LNP/GFP mRNA and Lipid 58 LNP/GFP mRNA recorded degeneration scores of <0.5 and ˜1.0, respectively, and their corresponding OCT images in FIG. 15B and FIG. 15F (using FIG. 15A vehicle as a reference) corroboratively indicate that the ONL layer maintained a healthy thickness. At day 1, none of the samples exhibited any retinal degradation (see FIGS. 14A-14F), using FIG. 14A vehicle as a reference.

FIGS. 16A-16D show the images of OCT (taken at day 22) and hematoxylin and eosin (H&E) qualitative analysis (taken at day 28) for the vehicle control and the low dose of 6 μg.

FIGS. 17A-17C are immunohistochemistry (IHC) images taken of the untreated area that served as negative control (FIG. 17A), the 6 μg low dose treatment (FIG. 17B), and the 30 μg high dose (FIG. 17C), 24 hours post-treatment.

### DETAILED DESCRIPTION

AAV vectors are currently the viral vector of choice for retinal gene transfer. However, the optimal method to deliver these treatments to the retinal pigment epithelial (RPE) cells and/or photoreceptor cells remains to be improved to increase transduction efficacy and to reduce complications associated with the highly invasive surgery required for subretinal injection of the viral vector suspension. The present disclosure describes for the first time the combination of a lipid nanoparticle, such as a lipid nanoparticle having Lipid A as described herein as an ionizable or cationic lipid, with an mRNA cargo for retinal delivery. Using mouse, rat, and non-human primate (NHP) in vivo systems, the present disclosure demonstrated that using LNP-delivered mRNA cargo, robust transgene expression can be achieved in the eye cup (RPE) and the neural retina, where the photoreceptors are. Importantly, the data presented herein showed that saturation was achieved at a low dose of LNP-delivered mRNA, thereby successfully achieving an excellent therapeutic index and tolerability of potential therapy Previously, when LNPs have been used for retinal gene transduction, the majority of the expression has been seen in the retinal pigmented epithelium (RPE) (Patel et al., Journal of Controlled Release Volume 303, 10 Jun. 2019, Pages 91-100) in the eyecup, while getting into the actual photoreceptors in the retina has remained a significant challenge. The results presented in the present disclosure surprisingly demonstrate not only that the LNPs are able to get into eye cup with expression equal to that of the RPE, but did so at a low dose.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), Fields Virology, 6th Edition, published by Lippincott Williams & Wilkins, Philadelphia, PA, USA (2013), Knipe, D. M. and Howley, P.M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used in this specification and the appended claims, the singular forms “a”, “an” and “the” include plural references unless the content clearly dictates otherwise.

The abbreviation, “e.g.” is derived from the Latin exempli gratia and is used herein to indicate a non-limiting example. Thus, the abbreviation “e.g.” is synonymous with the term “for example.”

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of 20% or +10%, more preferably +5%, even more preferably +1%, and still more preferably +0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

As used herein the terms, “administration,” “administering” and variants thereof refers to introducing a composition or agent (e.g., nucleic acids, in particular ceDNA) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrase “anti-therapeutic nucleic acid immune response”, “anti-transfer vector immune response”, “immune response against a therapeutic nucleic acid”, “immune response against a transfer vector”, or the like refers to any undesired immune response against a therapeutic nucleic acid, viral or non-viral in its origin. In some embodiments, the undesired immune response is an antigen-specific immune response against the viral transfer vector itself. In some embodiments, the immune response is specific to the transfer vector which can be double stranded DNA, single stranded RNA, or double stranded RNA. In other embodiments, the immune response is specific to a sequence of the transfer vector. In other embodiments, the immune response is specific to the CpG content of the transfer vector.

As used herein, the term “aqueous solution” refers to a composition comprising in whole, or in part, water.

As used herein, the term “bases” includes purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the terms “carrier” and “excipient” are meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “ceDNA” refers to capsid-free closed-ended linear double stranded (ds) duplex DNA for non-viral gene transfer, synthetic or otherwise. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministering DNA. According to some embodiments, the ceDNA is a dumbbell shaped linear duplex closed-ended DNA comprising two hairpin structures of ITRs in the 5′ and 3′ ends of an expression cassette. According to some embodiments, the ceDNA is a doggybone™ DNA. Detailed description of ceDNA is described in International Patent Application No. PCT/US2017/020828, filed Mar. 3, 2017, the entire contents of which are expressly incorporated herein by reference. Certain methods for the production of ceDNA comprising vanous inverted terminal repeat (ITR) sequences and configurations using cell-based methods are described in Example 1 of International Patent Application Nos. PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and are meant to refer to an invertebrate host cell (including, but not limited to an insect cell (e.g., an Sf9 cell)) infected with a ceDNA-baculovirus.

As used herein, the term “ceDNA genome” refers to an expression cassette that further incorporates at least one inverted terminal repeat (ITR) region. A ceDNA genome may further comprise one or more spacer regions. In some embodiments the ceDNA genome is incorporated as an intermolecular duplex polynucleotide of DNA into a plasmid or viral genome.

As used herein, the terms “DNA regulatory sequences,” “control elements,” and “regulatory elements,” are used interchangeably herein, and are meant to refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

The “ITR” can be artificially synthesized using a set of oligonucleotides comprising one or more desirable functional sequences (e.g., palindromic sequence, RBS). The ITR sequence can be an AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, ITR can be present in only one of end of the linear vector. For example, the ITR can be present on the 5′ end only. Some other cases, the ITR can be present on the 3′ end only in synthetic AAV vector. For convenience herein, an ITR located 5′ to (“upstream of”) an expression cassette in a synthetic AAV vector is referred to as a “5′ ITR” or a “left ITR”, and an ITR located 3′ to (“downstream of”) an expression cassette in a vector or synthetic AAV is referred to as a “3′ ITR” or a “right ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR″ are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single stranded AAV genome that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5′ ITR” or a “left ITR”, and an ITR located 3′ to (downstream of) an expression cassette in a synthetic AAV vector is referred to as a “3′ ITR” or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization -that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same R″shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the phrase an “effective amount” or “therapeutically effective amount” of an active agent or therapeutic agent, such as a therapeutic nucleic acid, is an amount sufficient to produce the desired effect, e.g., inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the term “expression” refers to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “expression vector” refers to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The expression vector may be a recombinant vector.

As used herein, the term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “spacer region” refers to an intervening sequence that separates functional elements in a vector or genome. In some embodiments, spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of base pair.

As used herein, the terms “expression cassette” and “expression unit” are used interchangeably, and meant to refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, the phrase “genetic disease” or “genetic disorder” refers to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

As used herein, the term “polypeptide” refers to a repeating sequence of amino acids.

As used herein, the term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and p-acyloxyacids, are also within the group designated as amphipathic lipids. Additionally, the amphipathic lipids described above can be mixed with other lipids including triglycerides and sterols.

In one embodiment, the lipid compositions comprise one or more tertiary amino groups, one or more phenyl ester bonds, and a disulfide bond.

As used herein, the term “lipid conjugate” refers to a conjugated lipid that inhibits aggregation of lipid particles (e.g., lipid nanoparticles). Such lipid conjugates include, but are not limited to, PEGylated lipids such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in International Patent Application Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

As used herein, the term “lipid encapsulated” refers to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the terms “lipid particle” or “lipid nanoparticle” refers to a lipid formulation that can be used to deliver a therapeutic agent such as nucleic acid therapeutics to a target site of interest (e.g., cell, tissue, organ, and the like). In one embodiment, the lipid particle of the disclosure is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In one embodiment, the lipid particle comprises a nucleic acid (e.g., ceDNA) and a lipid comprising one or more a tertiary amino groups, one or more phenyl ester bonds and a disulfide bond.

According to some embodiments, the lipid particles of the disclosure typically have a mean diameter of from about 20 nm to about 75 nm, about 20 nm to about 70 nm, about 25 nm to about 75 nm, about 25 nm to about 70 nm, from about 30 nm to about 75 nm, from about 30 nm to about 70 nm, from about 35 nm to about 75 nm, from about 35 nm to about 70 nm, from about 40 nm to about 75 nm, from about 40 nm to about 70 nm, from about 45 nm to about 75 nm, from about 50 nm to about 75 nm, from about 50 nm to about 70 nm, from about 60 nm to about 75 nm, from about 60 nm to about 70 nm, from about 65 nm to about 75 nm, from about 65 nm to about 70 nm, or about 20 nm, about 25 nm, about 30 nm, about 35 nm, about 40 nm, about 45 nm, about 50 nm, about 51 nm, about 52 nm, about 53 nm, about 54 nm, about 55 nm, about 56 nm, about 57 nm, about 58 nm, about 59 nm about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, or about 75 nm (+3 nm) in size.

Generally, the lipid particles (e.g., lipid nanoparticles) of the disclosure have a mean diameter selected to provide an intended therapeutic effect.

According to some embodiments, the lipid particles of the disclosure typically have a mean diameter of less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm in size.

As used herein, the term “cationic lipid” refers to any lipid that is positively charged at physiological pH. The cationic lipid in the lipid particles may comprise, e.g., one or more cationic lipids such as 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), “SS-cleavable lipid”, or a mixture thereof. In some embodiments, a cationic lipid is also an ionizable lipid, i.e., an ionizable cationic lipid. Corresponding quaternary lipids of all cationic lipids described herein (i.e., where the nitrogen atom in the cationic moiety is protonated and has four substituents) are contemplated within the scope of this disclosure. Any cationic lipid described herein may be converted to corresponding quaternary lipids, for example, by treatment with chloromethane (CH₃Cl) in acetonitrile (CH₃CN) and chloroform (CHCl₃).

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

As used herein, the term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

As used herein, the term “ionizable lipid” refers to a lipid, e.g., cationic lipid, having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. In some embodiments, ionizable lipid may include “cleavable lipid” or “SS-cleavable lipid”.

As used herein, the term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” refers to any amphipathic lipid as well as any other neutral lipid or anionic lipid.

As used herein, the term “cleavable lipid” or “SS-cleavable lipid” refers to a lipid comprising a disulfide bond cleavable unit. Cleavable lipids may include cleavable disulfide bond (“ss”) containing lipid-like materials that comprise a pH-sensitive tertiary amine and self-degradable phenyl ester. For example, a SS-cleavable lipid can be an ss-OP lipid (COATSOME® SS-OP), an ss-M lipid (COATSOME® SS-M), an ss-E lipid (COATSOME® SS-E), an ss-EC lipid (COATSOME® SS-EC), an ss-LC lipid (COATSOME® SS-LC), an ss-OC lipid (COATSOME® SS-OC), and an ss-PalmE lipid (see, for example, Formulae I-IV), or a lipid described by Togashi et al., (2018) Journal of Controlled Release “A hepatic pDNA delivery system based on an intracellular environment sensitive vitaminE-scaffold lipid-like material with the aid of an anti-inflammatory drug” 279:262-270. Additional examples of cleavable lipids are described in U.S. Pat. Nos. 9,708,628, and 10,385,030, the entire contents of which are incorporated herein by reference. In one embodiment, cleavable lipids comprise a tertiary amine, which responds to an acidic compartment, e.g., an endosome or lysosome for membrane destabilization and a disulfide bond that can be cleaved in a reducing environment, such as the cytoplasm. In one embodiment, a cleavable lipid is a cationic lipid. In one embodiment, a cleavable lipid is an ionizable cationic lipid. Cleavable lipids are described in more detail herein.

As used herein, the term “organic lipid solution” refers to a composition comprising in whole, or in part, an organic solvent having a lipid.

As used herein, the term “liposome” refers to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “local delivery” refers to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

As used herein, the term “nucleic acid,” refers to a polymer containing at least two nucleotides (i.e., deoxyribonucleotides or ribonucleotides) in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone™ DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, gRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, DOGGYBONE™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, “nucleotides” contain a sugar deoxyribose (DNA) or ribose (RNA), a base, and a phosphate group. Nucleotides are linked together through the phosphate groups.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “gap” refers to a discontinued portion of synthetic DNA vector of the present invention, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

The term “receptor” as used herein is intended to encompass the entire receptor or ligand-binding portions thereof. These portions of the receptor particularly include those regions sufficient for specific binding of the ligand to occur.

As used herein, the term “ocular disorder” is meant to include conditions associated with ocular angiogenesis, dry eye, inflammatory conditions, ocular hypertension and ocular diseases associated with elevated intraocular pressure (IOP), such as glaucoma.

As used herein, the term “subject” refers to a human or animal, to whom treatment, including prophylactic treatment, with the therapeutic nucleic acid according to the present disclosure, is provided. Usually, the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the phrase “subject in need” refers to a subject that (i) will be administered a ceDNA lipid particle (or pharmaceutical composition comprising a ceDNA lipid particle) according to the described disclosure, (ii) is receiving a ceDNA lipid particle (or pharmaceutical composition comprising a ceDNA lipid particle) according to the described disclosure; or (iii) has received a ceDNA lipid particle (or pharmaceutical composition comprising a ceDNA lipid particle) according to the described disclosure, unless the context and usage of the phrase indicates otherwise.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the term “systemic delivery” refers to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles (e.g., lipid nanoparticles) can be by any means known in the art including, for example, intravenous, subcutaneous, and intraperitoneal. In a preferred embodiment, systemic delivery of lipid particles (e.g., lipid nanoparticles) is by intravenous delivery.

As used herein, the term “terminal repeat” or “TR″ includes any viral or non-viral terminal repeat or synthetic sequence that comprises at least one minimal required origin of replication and a region comprising a palindromic hairpin structure. A Rep-binding sequence (“RBS” or also referred to as Rep-binding element (RBE)) and a terminal resolution site (“TRS”) together constitute a “minimal required origin of replication” for an AAV and thus the TR comprises at least one RBS and at least one TRS. TRs that are the inverse complement of one another within a given stretch of polynucleotide sequence are typically each referred to as an “inverted terminal repeat” or “ITR″. In the context of a virus, ITRs plays a critical role in mediating replication, viral particle and DNA packaging, DNA integration and genome and provirus rescue. TRs that are not inverse complement (palindromic) across their full length can still perform the traditional functions of ITRs, and thus, the term ITR is used to refer to a TR in an viral or non-viral AAV vector that is capable of mediating replication of in the host cell. It will be understood by one of ordinary skill in the art that in a complex AAV vector configurations more than two ITRs or asymmetric ITR pairs may be present.

As used herein, the terms “therapeutic amount”, “therapeutically effective amount”, an “amount effective”, or “pharmaceutically effective amount” of an active agent (e.g., a ceDNA lipid particle as described herein) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “alkyl” refers to a saturated monovalent hydrocarbon radical of 1 to 20 carbon atoms (i.e., C₁₋₂₀ alkyl). “Monovalent” means that alkyl has one point of attachment to the remainder of the molecule. In one embodiment, the alkyl has 1 to 12 carbon atoms (i.e., C₁₋₁₂ alkyl) or 1 to 10 carbon atoms (i.e., C₁₋₁₀ alkyl). In one embodiment, the alkyl has 1 to 8 carbon atoms (i.e., C₁₋₈ alkyl), 1 to 7 carbon atoms (i.e., C₁₋₇ alkyl), 1 to 6 carbon atoms (i.e., C₁₋₆ alkyl), 1 to 4 carbon atoms (i.e., C₁₋₄ alkyl), or 1 to 3 carbon atoms (i.e., C₁₋₃ alkyl). Examples include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. A linear or branched alkyl, such as a “linear or branched C₁₋₆ alkyl,” “linear or branched C₁₋₄ alkyl,” or “linear or branched C₁₋₃ alkyl” means that the saturated monovalent hydrocarbon radical is a linear or branched chain. As used herein, the term “linear” as referring to aliphatic hydrocarbon chains means that the chain is unbranched.

The term “alkylene” as used herein refers to a saturated divalent hydrocarbon radical of 1 to 20 carbon atoms (i.e., C₁₋₂₀ alkylene), examples of which include, but are not limited to, those having the same core structures of the alkyl groups as exemplified above. “Divalent” means that the alkylene has two points of attachment to the remainder of the molecule. In one embodiment, the alkylene has 1 to 12 carbon atoms (i.e., C₁₋₁₂ alkylene) or 1 to 10 carbon atoms (i.e., C₁₋₁₀ alkylene). In one embodiment, the alkylene has 1 to 8 carbon atoms (i.e., C₁₋ₛ alkylene), 1 to 7 carbon atoms (i.e., C₁₋₇ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), ethylene, or methylene. A linear or branched alkylene, such as a “linear or branched C₁₋₆ alkylene,” “linear or branched C₁₋₄ alkylene,” or “linear or branched C₁₋₃ alkylene” means that the saturated divalent hydrocarbon radical is a linear or branched chain.

The term “alkenyl” refers to straight or branched aliphatic hydrocarbon radical with one or more (e.g., one or two) carbon-carbon double bonds, wherein the alkenyl radical includes radicals having “cis” and “trans” orientations, or by an alternative nomenclature, “E” and “Z” orientations. [00126]“Alkenylene” as used herein refers to aliphatic divalent hydrocarbon radical of 2 to 20 carbon atoms (i.e., C₂₋₂₀ alkenylene) with one or two carbon-carbon double bonds, wherein the alkenylene radical includes radicals having “cis” and “trans” orientations, or by an alternative nomenclature, “E” and “Z” orientations. “Divalent” means that alkenylene has two points of attachment to the remainder of the molecule. In one embodiment, the alkenylene has 2 to 12 carbon atoms (i.e., C₂₋₁₆ alkenylene), 2 to 10 carbon atoms (i.e., C₂₋₁₀ alkenylene). In one embodiment, the alkenylene has 2 to four carbon atoms (C₂₋₄). Examples include, but are not limited to, ethylenylene or vinylene (—CH═CH—), allyl (—CH₂CH═CH—), and the like. A linear or branched alkenylene, such as a “linear or branched C₂₋₆ alkenylene,” “linear or branched C₂₋₄ alkenylene,” or “linear or branched C₂₋₃ alkenylene” means that the unsaturated divalent hydrocarbon radical is a linear or branched chain.

“Cycloalkylene” as used herein refers to a divalent saturated carbocyclic ring radical having 3 to 12 carbon atoms as a monocyclic ring, or 7 to 12 carbon atoms as a bicyclic ring. “Divalent” means that the cycloalkylene has two points of attachment to the remainder of the molecule. In one embodiment, the cycloalkylene is a 3- to 7-membered monocyclic or 3- to 6-membered monocyclic. Examples of monocyclic cycloalkyl groups include, but are not limited to, cyclopropylene, cyclobutylene, cyclopentylene, cyclohexylene, cycloheptylene, cyclooctylene, cyclononylene, cyclodecylene, cycloundecylene, cyclododecylene, and the like. In one embodiment, the cycloalkylene is cyclopropylene.

The terms “heterocycle,” “heterocyclyl,” heterocyclic and “heterocyclic ring” are used interchangeably herein and refer to a cyclic group which contains at least one N atom has a heteroatom and optionally 1-3 additional heteroatoms selected from N and S, and are non-aromatic (i.e., partially or fully saturated). It can be monocyclic or bicyclic (bridged or fused). Examples of heterocyclic rings include, but are not limited to, aziridinyl, diaziridinyl, thiaziridinyl, azetidinyl, diazetidinyl, triazetidinyl, thiadiazetidinyl, thiazetidinyl, pyrrolidinyl, pyrazolidinyl, imidazolinyl, isothiazolidinyl, thiazolidinyl, piperidinyl, piperazinyl, hexahydropyrimidinyl, azepanyl, azocanyl, and the like. The heterocycle contains 1 to 4 heteroatoms, which may be the same or different, selected from N and S. In one embodiment, the heterocycle contains 1 to 3 N atoms. In another embodiment, the heterocycle contains 1 or 2 N atoms. In another embodiment, the heterocycle contains 1 N atom. A “4- to 8-membered heterocyclyl” means a radical having from 4 to 8 atoms (including 1 to 4 heteroatoms selected from N and S, or 1 to 3 N atoms, or 1 or 2 N atoms, or 1 N atom) arranged in a monocyclic ring. A “5- or 6-membered heterocyclyl” means a radical having from 5 or 6 atoms (including 1 to 4 heteroatoms selected from N and S, or 1 to 3 N atoms, or 1 or 2 N atoms, or 1 N atom) arranged in a monocyclic ring. The term “heterocycle” is intended to include all the possible isomeric forms. Heterocycles are described in Paquette, Leo A., Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs (John Wiley & Sons, New York, 1950 to present), in particular Volumes 13, 14, 16, 19, and 28; and J. Am. Chem. Soc. (1960) 82:5566. The heterocyclyl groups may be carbon (carbon-linked) or nitrogen (nitrogen-linked) attached to the rest of the molecule where such is possible.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent.

Suitable substituents for an alkyl, alkylene, alkenylene, cycloalkylene, and heterocyclyl, are those which do not significantly adversely affect the biological activity of the bifunctional compound. Unless otherwise specified, exemplary substituents for these groups include linear, branched or cyclic alkyl, alkenyl or alkynyl having from 1 to 10 carbon atoms, aryl, heteroaryl, heterocyclyl, halogen, guanidinium [—NH(C═NH)NH₂], —OR₁₀₀, NR₁₀₁R₁₀₂, —NO₂, —NR₁₀₁COR₁₀₂, —SR₁₀₀, a sulfoxide represented by —SOR₁₀₁, a sulfone represented by —SO₂R₁₀₁, a sulfonate —SO₃M, a sulfate —OSO₃M, a sulfonamide represented by —SO₂NR₁₀₁R₁₀₂, cyano, an azido, —COR₁₀₁, —OCOR₁₀₁, —OCONR₁₀₁R₁₀₂ and a polyethylene glycol unit (—OCH₂CH₂)nR₁₀₁ wherein M is H or a cation (such as Na⁺ or K⁺); R₁₀₁, R₁₀₂ and R₁₀₃ are each independently selected from H, linear, branched or cyclic alkyl, alkenyl or alkynyl having from 1 to 10 carbon atoms, a polyethylene glycol unit (—OCH₂CH₂)ₙ-R₁₀₄, wherein n is an integer from 1 to 24, an aryl having from 6 to 10 carbon atoms, a heterocyclic ring having from 3 to 10 carbon atoms and a heteroaryl having 5 to 10 carbon atoms; and R₁₀₄ is H or a linear or branched alkyl having 1 to 4 carbon atoms, wherein the alkyl, alkenyl, alkynyl, aryl, heteroaryl and heterocyclcyl in the groups represented by R₁₀₀, R₁₀₁, R₁₀₂, R₁₀₃ and R₁₀₄ are optionally substituted with one or more (e.g., 2, 3, 4, 5, 6 or more) substituents independently selected from halogen, —OH, —CN, —NO₂, and unsubstituted linear or branched alkyl having 1 to 4 carbon atoms. Preferably, the substituent for the optionally substituted alkyl, alkylene, alkenylene, cycloalkylene, and heterocyclyl described above is selected from the group consisting of halogen, —CN, —NR₁₀₁R₁₀₂, —CF₃, —OR₁₀₀, aryl, heteroaryl, heterocyclyl, —SR₁₀₁, —SOR₁₀₁, —SO₂R₁₀₁, and —SO₃M. Alternatively, the suitable substituent is selected from the group consisting of halogen, —OH, —NO₂, —CN, C₁₋₄ alkyl, —OR₁₀₀, NR₁₀₁R₁₀₂, —NR₁₀₁COR₁₀₂, —SR₁₀₀, —SO₂R₁₀₁, —SO₂NR₁₀₁R₁₀₂, —COR₁₀₁, —OCOR₁₀₁, and —OCONR₁₀₁R₁₀₂, wherein R₁₀₀, R₁₀₁, and R₁₀₂ are each independently —H or C₁₋₄ alkyl.

“Halogen” as used herein refers to F, Cl, Br or I. “Cyano” is —CN.

“Amine” or “amino” as used herein interchangeably refers to a functional group that contains a basic nitrogen atom with a lone pair.

The term “pharmaceutically acceptable salt” as used herein refers to pharmaceutically acceptable organic or inorganic salts of an ionizable lipid of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited in any manner to the particular methodology, protocols, and reagents, etc., described herein and as such can vary. The terminology used herein is for the purpose of describing particular embodiments only and is not intended to limit the scope of the present invention, which is defined solely by the claims.

### II. Lipid Nanoparticle (LNP) Compositions for Retinal Delivery

Provided herein are compositions, e.g., pharmaceutical compositions, comprising a lipid nanoparticle (LNP), a therapeutic nucleic acid (TNA) and at least one pharmaceutically acceptable excipient, wherein the LNP comprises at least one lipid, and wherein the LNP is capable of delivering the TNA to a retinal cell. According to some embodiments, the LNP is capable of delivering the TNA to a photoreceptor (PR) cell. According to some embodiments, the LNP is capable of delivering the TNA to a retinal pigment epithelium (RPE) cell. According to some embodiments, the LNP is capable of delivering the TNA to a photoreceptor (PR) cell and a retinal pigment epithelium (RPE) cell, wherein expression of the TNA in the PR cell and expression of the TNA in RPE cell is evenly distributed.

As discussed above, the compositions, e.g., pharmaceutical compositions, comprising a LNP described herein surprisingly and advantageously were able to get into the neural retina (photoreceptor cells) with expression equal to that of the RPE, but did so at a low dose.

In one embodiment, a composition, e.g., pharmaceutical composition, described herein does not comprise any targeting ligand, such as an ApoE polypeptide, or a fragment thereof, and/or an ApoB polypeptide, or fragment thereof.

### A. Cationic Lipids

In some embodiments, the lipid nanoparticle having mean diameter of 20-74 nm comprises a cationic lipid. In some embodiments, the cationic lipid is, e.g., a non-fusogenic cationic lipid. By a “non-fusogenic cationic lipid” is meant a cationic lipid that can condense and/or encapsulate the nucleic acid cargo, such as ceDNA, but does not have, or has very little, fusogenic activity.

In some embodiments, the cationic lipid is described in the international and U.S. patent application publications listed below in Table 1, and determined to be non-fusogenic, as measured, for example, by a membrane-impermeable fluorescent dye exclusion assay, e.g., the assay described in the Examples section herein. Contents of all of these patent documents international and U.S. patent application publications listed below in Table 1 are incorporated herein by reference in their entireties.

TABLE 1
Exemplary patent documents describing cationic or ionizable lipids

| International Patent | U.S. Patent Application |
|---|---|
| Application Publication No. | Publication No. |
| WO2015/095340 | US2016/0311759 |
| WO2015/199952 | US2015/0376115 |
| WO2018/011633 | US2016/0151284 |
| WO2017/049245 | US2017/0210697 |
| WO2015/061467 | US2015/0140070 |
| WO2012/040184 | US2013/0178541 |
| WO2012/000104 | US2013/0303587 |
| WO2015/074085 | US2015/0141678 |
| WO2016/081029 | US2015/0239926 |
| WO2017/004143 | US2016/0376224 |
| WO2017/075531 | US2017/0119904 |
| WO2017/117528 | |
| WO2011/022460 | US2012/0149894 |
| WO2013/148541 | US2015/0057373 |
| WO2013/116126 | |
| WO2011/153120 | US2013/0090372 |
| WO2012/044638 | US2013/0274523 |
| WO2012/054365 | US2013/0274504 |
| WO2011/090965 | US2013/0274504 |
| WO2013/016058 | |
| WO2012/162210 | |
| WO2008/042973 | US2009/0023673 |
| WO2010/129709 | US2012/0128760 |
| WO2010/144740 | US201/003241240 |
| WO2012/099755 | US2014/0200257 |
| WO2013/049328 | US2015/0203446 |
| WO2013/086322 | US2018/0005363 |
| WO2013/086373 | US2014/0308304 |
| WO2011/071860 | US2013/0338210 |
| WO2009/132131 | |
| WO2010/048536 | |
| WO2010/088537 | US2012/0101148 |
| WO2010/054401 | US2012/0027796 |
| WO2010/054406 | |
| WO2010/054405 | |
| WO2010/054384 | US2012/0058144 |
| WO2012/016184 | US2013/0323269 |
| WO2009/086558 | US2011/0117125 |
| WO2010/042877 | US2011/0256175 |
| WO2011/000106 | US2012/0202871 |
| WO2011/000107 | US2011/0076335 |
| WO2005/120152 | US2006/0083780 |
| WO2011/141705 | US2013/0123338 |
| WO2013/126803 | US2015/0064242 |
| WO2006/07712 | US2006/0051405 |
| WO2011/038160 | US2013/0065939 |
| WO2005/121348 | US2006/0008910 |
| WO2011/066651 | US2003/0022649 |
| WO2009/127060 | US2010/0130588 |
| WO2011/141704 | US2013/0116307 |
| WO2006/069782 | US2010/0062967 |
| WO2012/031043 | US2013/0202684 |
| WO2013/006825 | US2014/0141070 |
| WO2013/033563 | US2014/0255472 |
| WO2013/089151 | US2014/0039032 |
| WO2017/099823 | US2018/0028664 |
| WO2015/095346 | US2016/0317458 |
| WO2013/086354 | US2013/0195920 |

In some embodiments, the cationic lipid is selected from the group consisting of N-[1-(2,3-dioleyloxy)propyll-N,N,N-trimethylammonium chloride (DOTMA); N-[1-(2,3-dioleoyloxy)propyll-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), Ni-[2-((1S)-1-[(3-aminopropyl)amino]-4-[di(3-amino-propyl) ammnolbutylcarboxamidoiethyl 1-3,4-di[oleyloxy]-benzamide(MVL5); Dioctadecylamido-glycylspermine (DOGS); 3b-[N-(N′,N′-dimethylaminoethyl)carbamoyl]cholesterol (DC-Chol); Dioctadecyldimethylammonium Bromide (DDAB); a Saint lipid (e.g., SAINT-2, N-methyl1-4-(dioleyl)methylpyridinium); 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); Di-alkylated Amino Acid (DILA2) (e.g., C18:1-norArg-C16); Dioleyldimethylammonium chloride (DODAC); 1-palmitoyl-2-oleoyl-sn-glycero-3-ethylpho sphocholine (POEPC); and 1,2-dimyristoleoyl-sn-glycero-3-ethylphosphocholine (MOEPC). In some variations, the condensing agent, e.g. a cationic lipid, is a lipid such as, e.g., Dioctadecyldimethylammonium bromide (DDAB), 1,2-dilinoleyloxy-3-dimethylaminopropane (DLinDMA), 2,2-dilinoley 1-4-(2dimethylaminoethyl)-[1,31-dioxolane (DLin-KC2-DMA), heptatriaconta-6,9,28,31-tetraen-19-yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diy1 dioleate hydrochloride (DODAPen-Cl), (R)-5-guanidinopentane-1,2-diyl dioleate hydrochloride (DOPen-G), (R)-N,N,N-trimethyl-4,5-bis(oleoyloxy)pentan-1-aminium chloride(DOTAPen).

In some embodiments, the condensing lipid is DOTAP.

### Ionizable Lipids

According to some embodiments, also provided herein are pharmaceutical compositions containing LNPs comprising an ionizable lipid and a therapeutic nucleic acid like non-viral vector (e.g., ceDNA). Such LNPs can be used to deliver, e.g., the pharmaceutical composition comprising a lipid nanoparticle (LNP) and a therapeutic nucleic acid (TNA), as described herein, to a target site of interest (e.g., cell, tissue, organ, and the like).

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/01 19904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/01 16307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

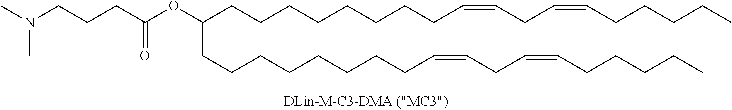 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, the contents of which is incorporated herein by reference in its entirety.

In some embodiments, the ionizable lipid is (13Z,16Z)-N,N-dimethyl-3-nonyldocosa-13,16-dien-1-amine (Compound 32), as described in WO2012/040184, the contents of which is incorporated herein by reference in its entirety.

In some embodiments, the ionizable lipid is Compound 6 or Compound 22 as described in WO2015/199952, the contents of which is incorporated herein by reference in its entirety. Formula (I)

According to some embodiments, the ionizable lipids are represented by Formula (I):

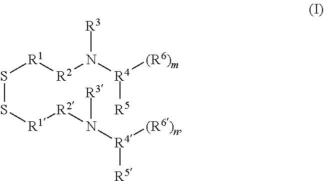

or a pharmaceutically acceptable salt thereof, wherein:
R¹ and R^{1′} are each independently C₁₋₃ alkylene;
R² and R^{2′} are each independently linear or branched C₁₋₆ alkylene, or C₃₋₆ cycloalkylene;
R³ and R^{3′} are each independently optionally substituted C₁₋₆ alkyl or optionally substituted C₃₋₆ cycloalkyl;
or alternatively, when R² is branched C₁₋₆ alkylene and when R³ is C₁₋₆ alkyl, R² and R³, taken together with their intervening N atom, form a 4- to 8-membered heterocyclyl;
or alternatively, when R^{2′} is branched C₁₋₆ alkylene and when R^{3′} is C₁₋₆ alkyl, R^{2′} and R³, taken together with their intervening N atom, form a 4- to 8-membered heterocyclyl;
R⁴ and R^{4′} are each independently —CH, —CH₂CH, or —(CH₂)₂CH;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

According to some embodiments of any of the aspects or embodiments herein, R² and R^{2′} are each independently C₁₋₃ alkylene.

According to some embodiments of any of the aspects or embodiments herein, the linear or branched C₁₋₃ alkylene represented by R¹ or R″, the linear or branched C₁₋₆ alkylene represented by R² or R^{2′}, and the optionally substituted linear or branched C₁₋₆ alkyl are each optionally substituted with one or more halo and cyano groups.

According to some embodiments of any of the aspects or embodiments herein, R¹ and R² taken together are C₁₋₃ alkylene and R^{1′} and R^{2′} taken together are C₁₋₃ alkylene, e.g., ethylene.

According to some embodiments of any of the aspects or embodiments herein, R³ and R^{3′} are each independently optionally substituted C₁₋₃ alkyl, e.g., methyl.

According to some embodiments of any of the aspects or embodiments herein, R⁴ and R^{4′} are each —CH.

According to some embodiments of any of the aspects or embodiments herein, R² is optionally substituted branched C₁₋₆ alkylene; and R² and R³, taken together with their intervening N atom, form a 5- or 6-membered heterocyclyl. According to some embodiments of any of the aspects or embodiments herein, R^{2′} is optionally substituted branched C₁₋₆ alkylene; and R^{2′} and R^{3′}, taken together with their intervening N atom, form a 5- or 6-membered heterocyclyl, such as pyrrolidinyl or piperidinyl.

According to some embodiments of any of the aspects or embodiments herein, R⁴ is —C(R^{a})₂CR^{a}, or -[C(R^{a})₂]₂CR^{a} and R^{a} is C₁₋₃ alkyl; and R³ and R⁴, taken together with their intervening N atom, form a 5- or 6-membered heterocyclyl. According to some embodiments of any of the aspects or embodiments herein, R^{4′} is —C(R^{a})₂CR^{a}, or -[C(R^{a})₂]₂CR^{a} and R^{a} is C₁₋₃ alkyl; and R^{3′} and R^{4′}, taken together with their intervening N atom, form a 5- or 6-membered heterocyclyl, such as pyrrolidinyl or piperidinyl.

According to some embodiments of any of the aspects or embodiments herein, R⁵ and R^{5′} are each independently C₁₋₁₀ alkylene or C₂₋₁₀ alkenylene. In one embodiment, R⁵ and R^{5′} are each independently C₁₋₈ alkylene or C₁₋₆ alkylene.

According to some embodiments of any of the aspects or embodiments herein, R⁶ and R^{6′}, for each occurrence, are independently C₁₋₁₀ alkylene, C₃₋₁₀ cycloalkylene, or C₂₋₁₀alkenylene. In one embodiment, C₁₋₆ alkylene, C₃₋₆ cycloalkylene, or C₂₋₆ alkenylene. In one embodiment the C₃₋₁₀ cycloalkylene or the C₃₋₆ cycloalkylene is cyclopropylene. According to some embodiments of any of the aspects or embodiments herein, m and n are each 3.

According to some embodiments of any of the aspects or embodiments herein, the ionizable lipid is selected from any one of the lipids in Table 2 or a pharmaceutically acceptable salt thereof.

TABLE 2
Exemplary ionizable lipids of Formula (I)

| Lipid No. | Structure and Name |
|---|---|
| 1 | 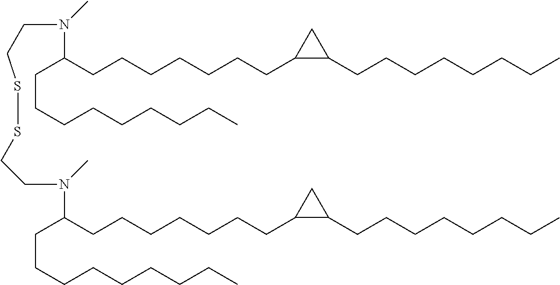 [SMILES: CCCCCCCCCC(CCCCCCCC1CC1CCCCCCCC)N(C)CCSSCCN(C)C(CCCCCCCCC)CCCCCCCC1CC1CCCCCCCC] |
| | N,N′-(disulfanediylbis(ethane-2,1-diyl))bis(N-methyl-1-(2- |
| | octylcyclopropyl)heptadecan-8-amine) |
| 2 | 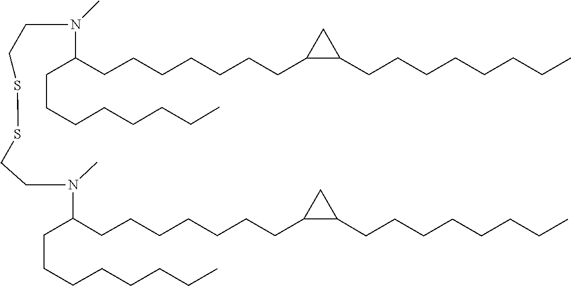 [SMILES: CCCCCCCCC1CC1CCCCCCCC(CCCCCCCC)N(C)CCSSCCN(C)C(CCCCCCCC)CCCCCCCC1CC1CCCCCCCC] |
| | N,N′-(disulfanediylbis(ethane-2,1-diyl))bis(N-methyl-1-(2- |
| | octylcyclopropyl)hexadecan-8-amine) |
| 3 | 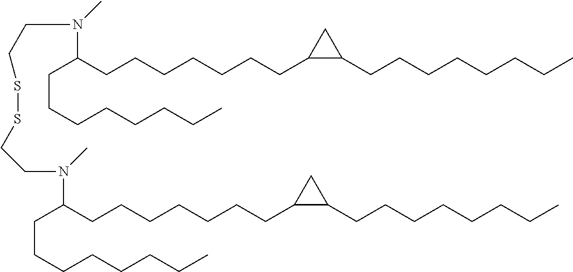 [SMILES: CCCCCCCCC1CC1CCCCCCCC(CCCCCCCC)N(C)CCSSCCN(C)C(CCCCCCCC)CCCCCCCC1CC1CCCCCCCC] |
| | N,N′-(disulfanediylbis(ethane-2,1-diyl))bis(N-methyl-1-(2- |
| | octylcyclopropyl)hexadecan-8-amine) |
| 4 | 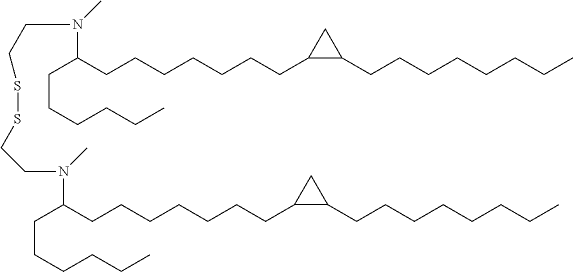 [SMILES: CCCCCCCCC1CC1CCCCCCCC(CCCCCC)N(C)CCSSCCN(C)C(CCCCCC)CCCCCCCC1CC1CCCCCCCC] |
| | N,N′-(disulfanediylbis(ethane-2,1-diyl))bis(N-methyl-14-(2- |
| | octylcyclopropyl)tetradecan-7-amine) |
| 5 | 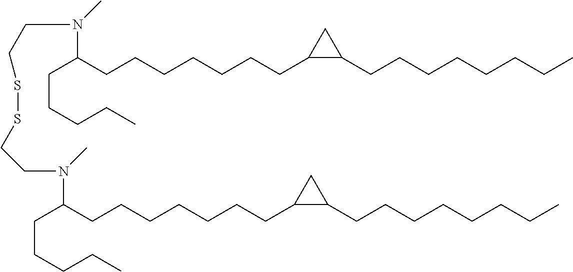 [SMILES: CCCCCCCCC1CC1CCCCCCCC(CCCCC)N(C)CCSSCCN(C)C(CCCCC)CCCCCCCC1CC1CCCCCCCC] |
| | N,N′-(disulfanediylbis(ethane-2,1-diyl))bis(N-methyl-13-(2- |
| | octylcyclopropyl)tridecan-6-amine) |
| 6 | 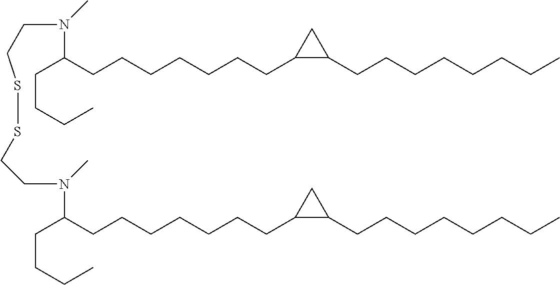 [SMILES: CCCCCCCCC1CC1CCCCCCCC(CCCC)N(C)CCSSCCN(C)C(CCCC)CCCCCCCC1CC1CCCCCCCC] |
| | N,N′-(disulfanediylbis(ethane-2,1-diyl))bis(N-methyl-12-(2- |
| | octylcyclopropyl)dodecan-5-amine) |
| 7 | 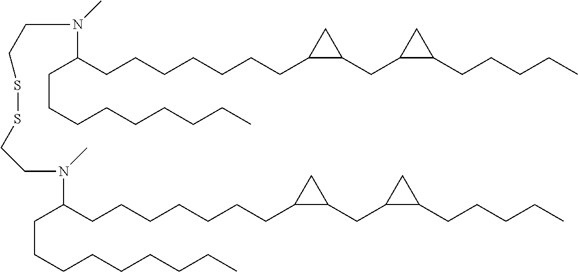 [SMILES: CCCCCCCCCC(CCCCCCCC1CC1CC1CC1CCCCC)N(C)CCSSCCN(C)C(CCCCCCCCC)CCCCCCCC1CC1CC1CC1CCCCC] |
| | N,N′-(disulfanediylbis(ethane-2,1-diyl))bis(N-methyl-1-(2-((2- |
| | pentylcyclopropyl)methyl)cyclopropyl)heptadecan-8-amine) |
| 8 | 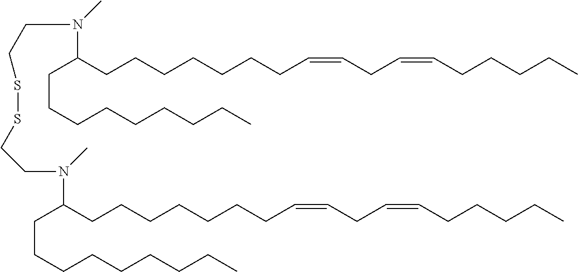 [SMILES: CCCCC/C=C\C/C=C\CCCCCCCC(CCCCCCCCC)N(C)CCSSCCN(C)C(CCCCCCC/C=C\C/C=C\CCCCC)CCCCCCCCC] |
| | (18Z,18′Z,21Z,21′Z)-N,N′-(disulfanediylbis(ethane-2,1-diyl))bis(N-methylheptacosa- |
| | 18,21-dien-10-amine) |
| 9 | 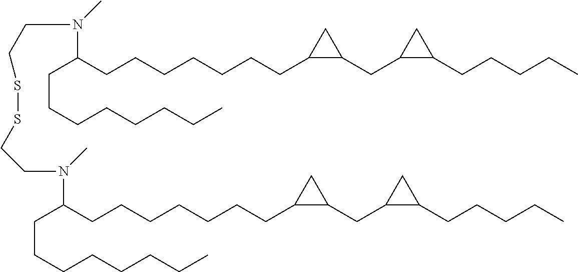 [SMILES: CCCCCCCCC(CCCCCCCC1CC1CC1CC1CCCCC)N(C)CCSSCCN(C)C(CCCCCCCC)CCCCCCCC1CC1CC1CC1CCCCC] |
| | N,N′-(disulfanediylbis(ethane-2,1-diyl))bis(N-methyl-1-(2-((2- |
| | pentylcyclopropyl)methyl)cyclopropyl)hexadecan-8-amine) |
| 10 | 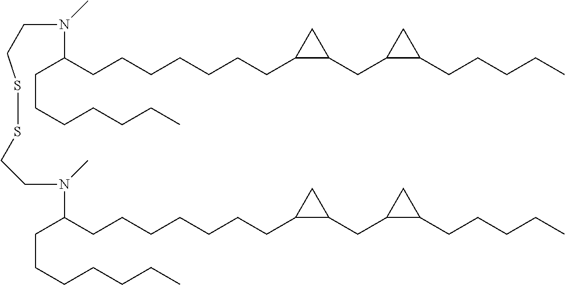 [SMILES: CCCCCCCC(CCCCCCCC1CC1CC1CC1CCCCC)N(C)CCSSCCN(C)C(CCCCCCC)CCCCCCCC1CC1CC1CC1CCCCC] |
| | N,N′-(disulfanediylbis(ethane-2,1-diyl))bis(N-methyl-1-(2-((2- |
| | pentylcyclopropyl)methyl)cyclopropyl)pentadecan-8-amine) |
| 11 | 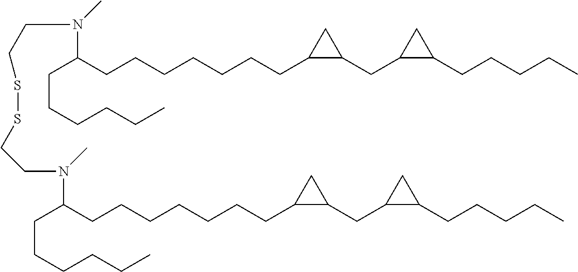 [SMILES: CCCCCCC(CCCCCCCC1CC1CC1CC1CCCCC)N(C)CCSSCCN(C)C(CCCCCC)CCCCCCCC1CC1CC1CC1CCCCC] |
| | N,N′-(disulfanediylbis(ethane-2,1-diyl))bis(N-methyl-14-(2-((2- |
| | pentylcyclopropyl)methyl)cyclopropyl)tetradecan-7-amine) |
| 12 | 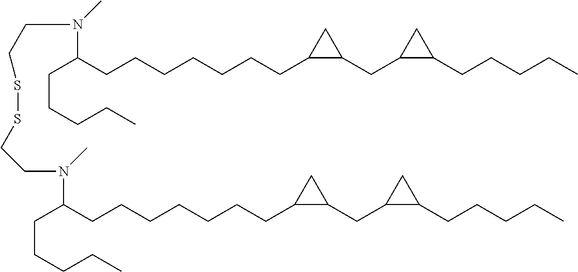 [SMILES: CCCCCC1CC1CC1CC1CCCCCCCC(CCCCC)N(C)CCSSCCN(C)C(CCCCC)CCCCCCCC1CC1CC1CC1CCCCC] |
| | N,N′-(disulfanediylbis(ethane-2,1-diyl))bis(N-methyl-13-(2-((2- |
| | pentylcyclopropyl)methyl)cyclopropyl)tridecan-6-amine) |
| 13 | 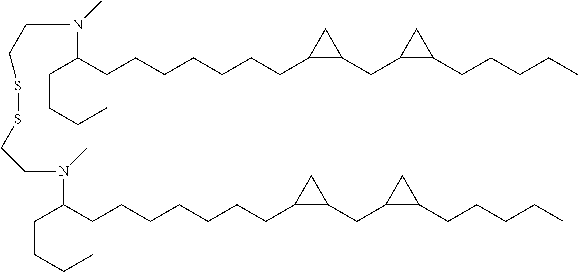 [SMILES: CCCCCC1CC1CC1CC1CCCCCCCC(CCCC)N(C)CCSSCCN(C)C(CCCC)CCCCCCCC1CC1CC1CC1CCCCC] |
| | N,N′-(disulfanediylbis(ethane-2,1-diyl))bis(N-methyl-12-(2-((2- |
| | pentylcyclopropyl)methyl)cyclopropyl)dodecan-5-amine) |
| 14 | 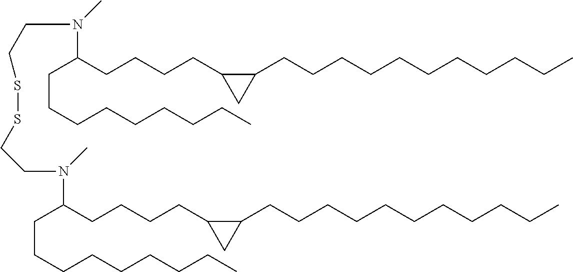 [SMILES: CCCCCCCCCCCC1CC1CCCCC(CCCCCCCCC)N(C)CCSSCCN(C)C(CCCCCCCCC)CCCCC1CC1CCCCCCCCCCC] |
| | N,N′-(disulfanediylbis(ethane-2,1-diyl))bis(N-methyl-1-(2- |
| | undecylcyclopropyl)tetradecan-5-amine) |
| 15 | 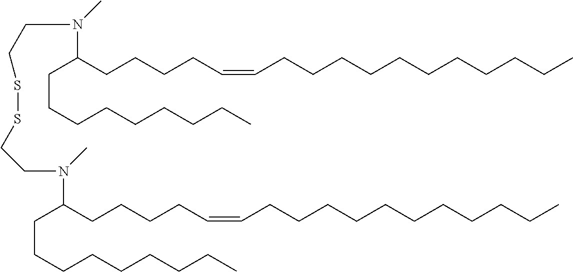 [SMILES: CCCCCCCCCCC/C=C\CCCCC(CCCCCCCCC)N(C)CCSSCCN(C)C(CCCC/C=C\CCCCCCCCCCC)CCCCCCCCC] |
| | (15Z,15′Z)-N,N′-(disulfanediylbis(ethane-2,1-diyl))bis(N-methylheptacos-15-en-10- |
| | amine) |
| 16 | 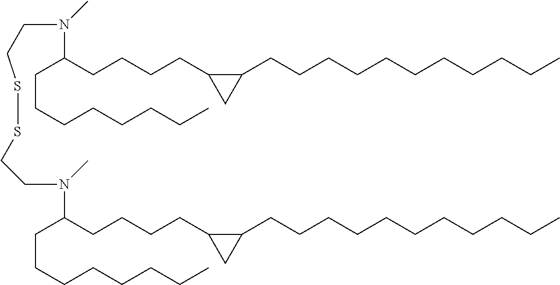 [SMILES: CCCCCCCCCCCC1CC1CCCCC(CCCCCCCC)N(C)CCSSCCN(C)C(CCCCCCCC)CCCCC1CC1CCCCCCCCCCC] |
| | N,N′-(disulfanediylbis(ethane-2,1-diyl))bis(N-methyl-1-(2- |
| | undecylcyclopropyl)tridecan-5-amine) |
| 17 | 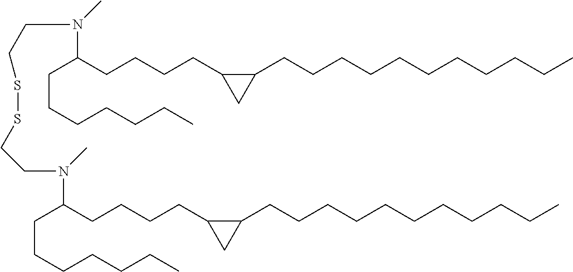 [SMILES: CCCCCCCCCCCC1CC1CCCCC(CCCCCCC)N(C)CCSSCCN(C)C(CCCCCCC)CCCCC1CC1CCCCCCCCCCC] |
| | N,N′-(disulfanediylbis(ethane-2,1-diyl))bis(N-methyl-1-(2- |
| | undecylcyclopropyl)dodecan-5-amine) |
| 18 | 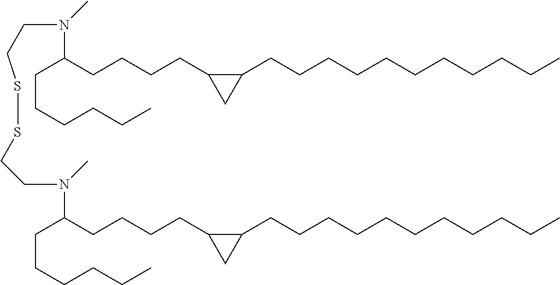 [SMILES: CCCCCCCCCCCC1CC1CCCCC(CCCCCC)N(C)CCSSCCN(C)C(CCCCCC)CCCCC1CC1CCCCCCCCCCC] |
| | N,N′-(disulfanediylbis(ethane-2,1-diyl))bis(N-methyl-1-(2- |
| | undecylcyclopropyl)undecan-5-amine) |
| 19 | 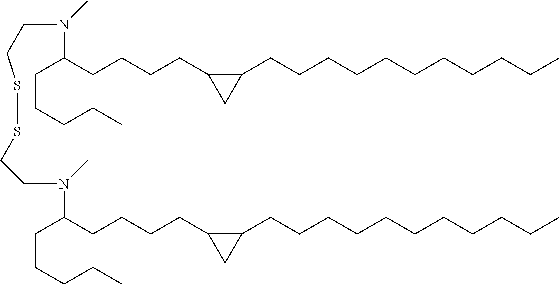 [SMILES: CCCCCCCCCCCC1CC1CCCCC(CCCCC)N(C)CCSSCCN(C)C(CCCCC)CCCCC1CC1CCCCCCCCCCC] |
| | N,N′-(disulfanediylbis(ethane-2,1-diyl))bis(N-methyl-1-(2- |
| | undecylcyclopropyl)decan-5-amine) |
| 20 | 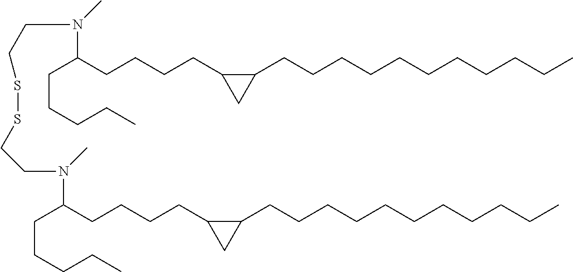 [SMILES: CCCCCCCCCCCC1CC1CCCCC(CCCCC)N(C)CCSSCCN(C)C(CCCCC)CCCCC1CC1CCCCCCCCCCC] |
| | N,N′-(disulfanediylbis(ethane-2,1-diyl))bis(N-methyl-1-(2- |
| | undecylcyclopropyl)decan-5-amine) |
| 21 | 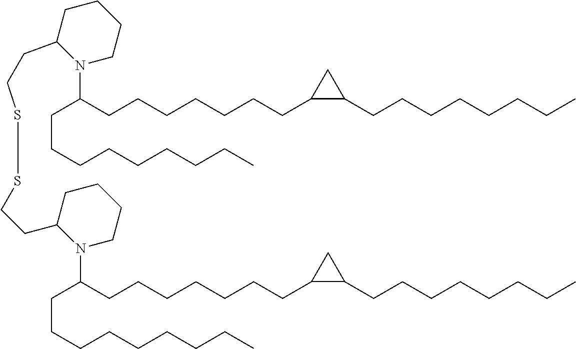 [SMILES: CCCCCCCCCC(CCCCCCCC1CC1CCCCCCCC)N1CCCCC1CCSSCCC1CCCCN1C(CCCCCCCCC)CCCCCCCC1CC1CCCCCCCC] |
| | 1,2-bis(2-(1-(1-(2-octylcyclopropyl)heptadecan-8-yl)piperidin-2-yl)ethyl)disulfane |
| 22 | 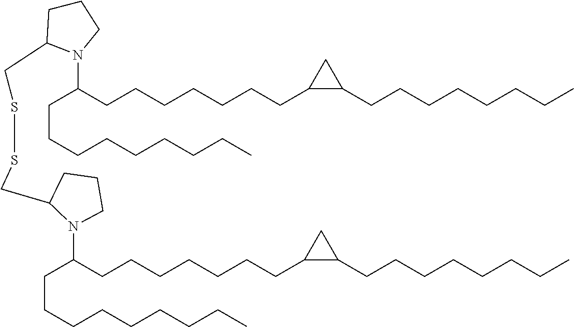 [SMILES: CCCCCCCCCC(CCCCCCCC1CC1CCCCCCCC)N1CCCC1CSSCC1CCCN1C(CCCCCCCCC)CCCCCCCC1CC1CCCCCCCC] |
| | 1,2-bis((1-(1-(2-octylcyclopropyl)heptadecan-8-yl)pyrrolidin-2-yl)methyl)disulfane |
| 23 | 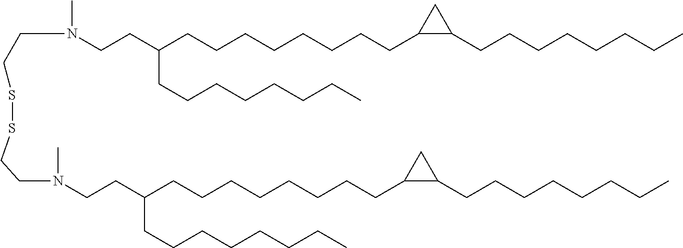 [SMILES: CCCCCCCCC(CCCCCCCCC1CC1CCCCCCCC)CCN(C)CCSSCCN(C)CCC(CCCCCCCC)CCCCCCCCC1CC1CCCCCCCC] |
| | N,N′-(disulfanediylbis(ethane-2,1-diyl))bis(N-methyl-3-octyl-11-(2- |
| | octylcyclopropyl)undecan-1-amine) |
| 24 | 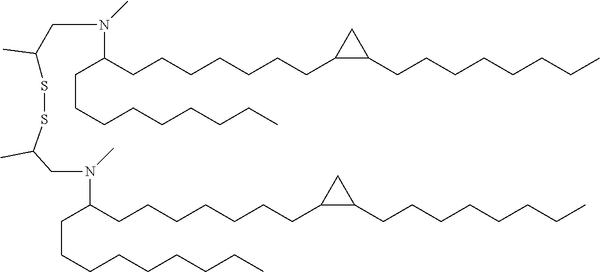 [SMILES: CCCCCCCCCC(CCCCCCCC1CC1CCCCCCCC)N(C)CC(C)SSC(C)CN(C)C(CCCCCCCCC)CCCCCCCC1CC1CCCCCCCC] |
| | N,N′-(disulfanediylbis(propane-2,1-diyl))bis(N-methyl-1-(2- |
| | octylcyclopropyl)heptadecan-9-amine) |
| 25 | 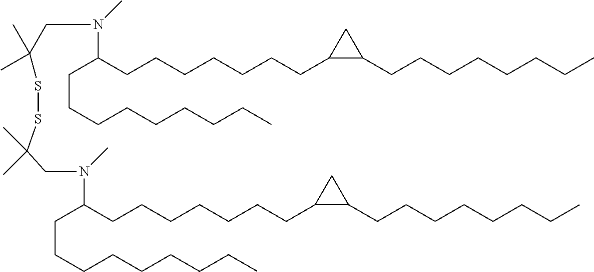 [SMILES: CCCCCCCCCC(CCCCCCCC1CC1CCCCCCCC)N(C)CC(C)(C)SSC(C)(C)CN(C)C(CCCCCCCCC)CCCCCCCC1CC1CCCCCCCC] |
| | N,N′-(disulfanediylbis(2-methylpropane-2,1-diyl))bis(N-methyl-1-(2- |
| | octylcyclopropyl)heptadecan-8-amine) |
| 26 | 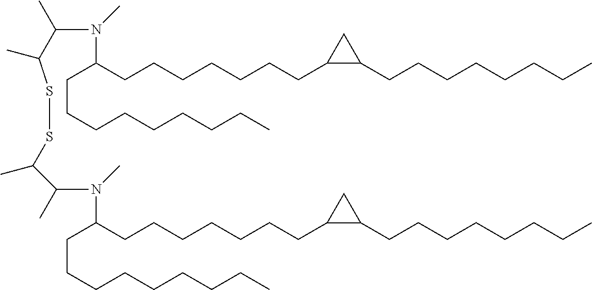 [SMILES: CCCCCCCCCC(CCCCCCCC1CC1CCCCCCCC)N(C)C(C)C(C)SSC(C)C(C)N(C)C(CCCCCCCCC)CCCCCCCC1CC1CCCCCCCC] |
| | N,N′-(disulfanediylbis(butane-3,2-diyl))bis(N-methyl-1-(2- |
| | octylcyclopropyl)heptadecan-8-amine) |
| 27 | 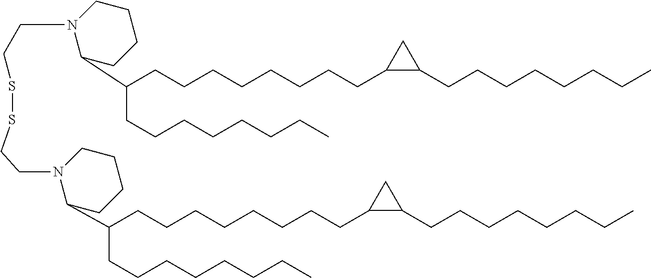 [SMILES: CCCCCCCCC1CC1CCCCCCCCC(CCCCCCCC)C1CCCCN1CCSSCCN1CCCCC1C(CCCCCCCC)CCCCCCCCC1CC1CCCCCCCC] |
| | 1,2-bis(2-(2-(1-(2-octylcyclopropyl)heptadecan-9-yl)piperidin-1-yl)ethyl)disulfane |
| 28 | 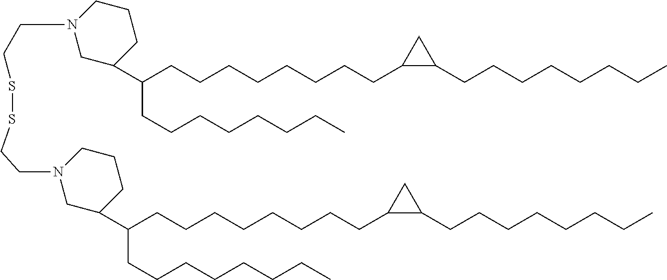 [SMILES: CCCCCCCCC(CCCCCCCCC1CC1CCCCCCCC)C1CCCN(CCSSCCN2CCCC(C(CCCCCCCC)CCCCCCCCC3CC3CCCCCCCC)C2)C1] |
| | 1,2-bis(2-(3-(1-(2-octylcyclopropyl)heptadecan-9-yl)piperidin-1-yl)ethyl)disulfane |
| 29 | 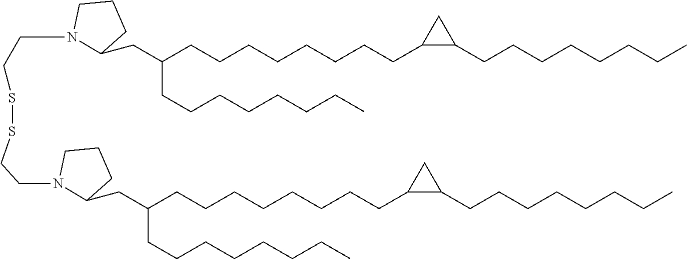 [SMILES: CCCCCCCCC(CCCCCCCCC1CC1CCCCCCCC)CC1CCCN1CCSSCCN1CCCC1CC(CCCCCCCC)CCCCCCCCC1CC1CCCCCCCC] |
| | 1,2-bis(2-(2-(2-octyl-10-(2-octylcyclopropyl)decyl)pyrrolidin-1-yl)ethyl)disulfane |
| 30 | 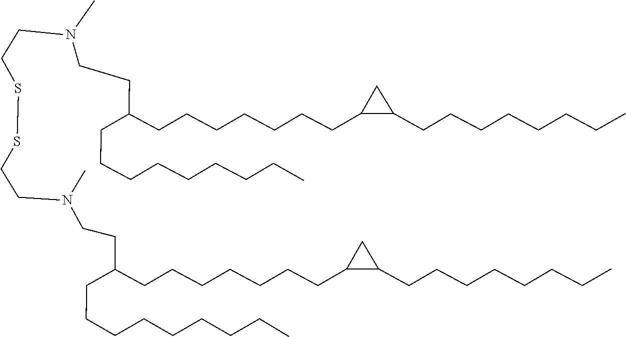 [SMILES: CCCCCCCCCC(CCCCCCCC1CC1CCCCCCCC)CCN(C)CCSSCCN(C)CCC(CCCCCCCCC)CCCCCCCC1CC1CCCCCCCC] |
| | N,N′-(disulfanediylbis(ethane-2,1-diyl))bis(N-methyl-3-(7-(2- |
| | octylcyclopropyl)heptyl)dodecan-1-amine) |
| 31 | 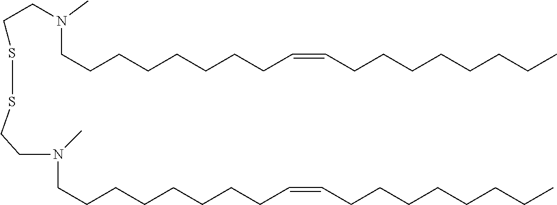 [SMILES: CCCCCCCC/C=C\CCCCCCCCN(C)CCSSCCN(C)CCCCCCCC/C=C\CCCCCCCC] |
| | (9Z,9′Z)-N,N′-(disulfanediylbis(ethane-2,1-diyl))bis(N-methyloctadec-9-en-1-amine) |
| 32 | 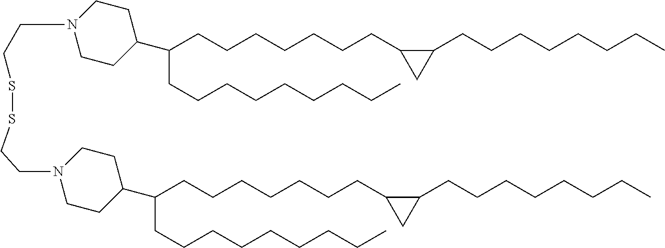 [SMILES: CCCCCCCCCC(CCCCCCCC1CC1CCCCCCCC)C1CCN(CCSSCCN2CCC(C(CCCCCCCCC)CCCCCCCC3CC3CCCCCCCC)CC2)CC1] |
| | 1,2-bis(2-(4-(1-(2-octylcyclopropyl)heptadecan-8-yl)piperidin-1-yl)ethyl)disulfane |
| 33 | 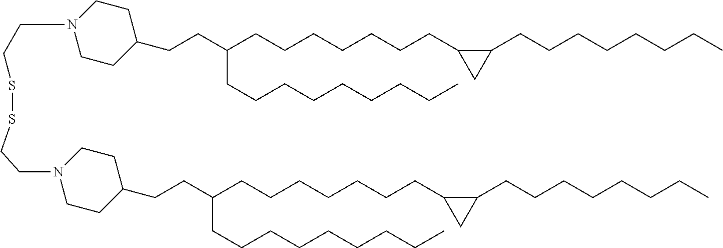 [SMILES: CCCCCCCCCC(CCCCCCCC1CC1CCCCCCCC)CCC1CCN(CCSSCCN2CCC(CCC(CCCCCCCCC)CCCCCCCC3CC3CCCCCCCC)CC2)CC1] |
| | 1,2-bis(2-(4-(3-(7-(2-octylcyclopropyl)heptyl)dodecyl)piperidin-1-yl)ethyl)disulfane |
| 34 | 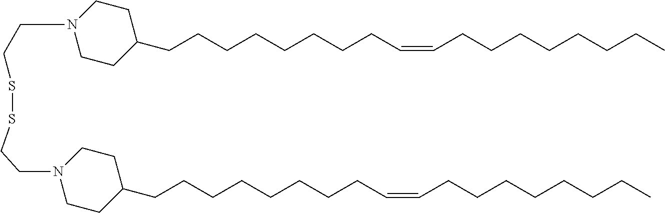 [SMILES: CCCCCCCC/C=C\CCCCCCCCC1CCN(CCSSCCN2CCC(CCCCCCCC/C=C\CCCCCCCC)CC2)CC1] |
| | 1,2-bis(2-(4-((Z)-octadec-9-en-1-yl)piperidin-1-yl)ethyl)disulfane |
| 35 | 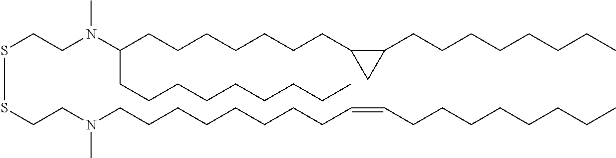 [SMILES: CCCCCCCC/C=C\CCCCCCCCN(C)CCSSCCN(C)C(CCCCCCCCC)CCCCCCCC1CC1CCCCCCCC] |
| | (Z)-N-methyl-N-(2-((2-(methyl(1-(2-octylcyclopropyl)heptadecan-8- |
| | yl)amino)ethyl)disulfaneyl)ethyl)octadec-9-en-1-amine |
| 36 | 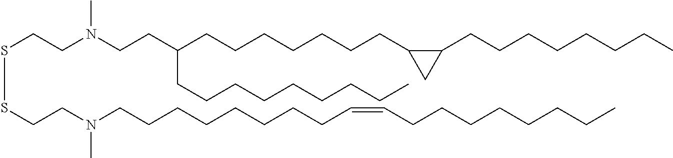 [SMILES: CCCCCCCC/C=C\CCCCCCCCN(C)CCSSCCN(C)CCC(CCCCCCCCC)CCCCCCCC1CC1CCCCCCCC] |
| | (Z)-N-methyl-N-(2-((2-(methyl(3-(7-(2- |
| | octylcyclopropyl)heptyl)dodecyl)amino)ethyl)disulfaneyl)ethyl)octadec-9-en-1- |
| | amine |
| 37 | 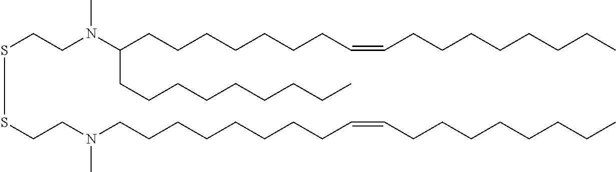 [SMILES: CCCCCCCC/C=C\CCCCCCCCN(C)CCSSCCN(C)C(CCCCCCC/C=C\CCCCCCCC)CCCCCCCCC] |
| | (Z)-N-methyl-N-(2-((2-(methyl((Z)-octadec-9-en-1- |
| | yl)amino)ethyl)disulfaneyl)ethyl)heptacos-18-en-10-amine |
| 38 | 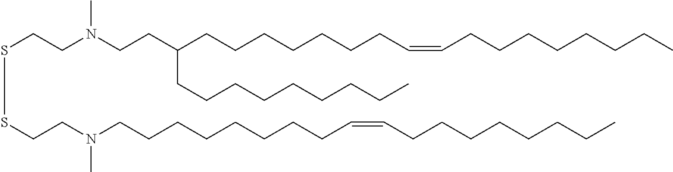 [SMILES: CCCCCCCC/C=C\CCCCCCCCN(C)CCSSCCN(C)CCC(CCCCCCC/C=C\CCCCCCCC)CCCCCCCCC] |
| | (Z)-N-methyl-N-(2-((2-(methyl((Z)-octadec-9-en-1- |
| | yl)amino)ethyl)disulfaneyl)ethyl)-3-nonylicos-11-en-1-amine |
| 39 | 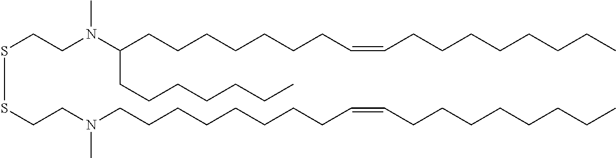 [SMILES: CCCCCCCC/C=C\CCCCCCCCN(C)CCSSCCN(C)C(CCCCCCC)CCCCCCC/C=C\CCCCCCCC] |
| | (Z)-N-methyl-N-(2-((2-(methyl((Z)-octadec-9-en-1- |
| | yl)amino)ethyl)disulfaneyl)ethyl)pentacos-16-en-8-amine |
| 40 | 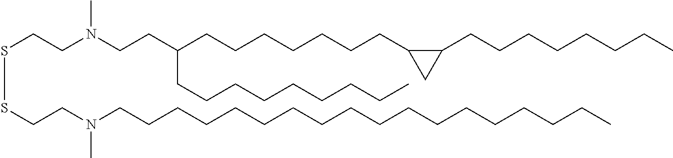 [SMILES: CCCCCCCCCCCCCCCCCCN(C)CCSSCCN(C)CCC(CCCCCCCCC)CCCCCCCC1CC1CCCCCCCC] |
| | N-methyl-N-(2-((2-(methyl(3-(7-(2- |
| | octylcyclopropyl)heptyl)dodecyl)amino)ethyl)disulfaneyl)ethyl)octadecan-1-amine |
| 41 | 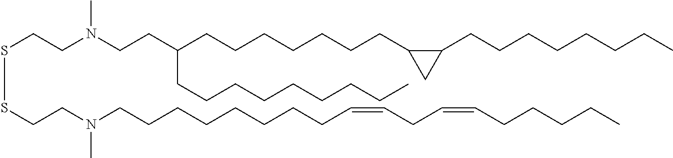 [SMILES: CCCCC/C=C\C/C=C\CCCCCCCCN(C)CCSSCCN(C)CCC(CCCCCCCCC)CCCCCCCC1CC1CCCCCCCC] |
| | (9Z,12Z)-N-methyl-N-(2-((2-(methyl(3-(7-(2- |
| | octylcyclopropyl)heptyl)dodecyl)amino)ethyl)disulfaneyl)ethyl)octadeca-9,12-dien- |
| | 1-amine |
| 42 | 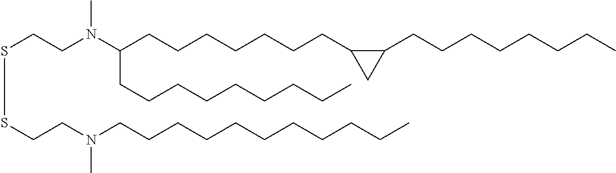 [SMILES: CCCCCCCCCCCN(C)CCSSCCN(C)C(CCCCCCCCC)CCCCCCCC1CC1CCCCCCCC] |
| | N-methyl-N-(2-((2-(methyl(undecyl)amino)ethyl)disulfaneyl)ethyl)-1-(2- |
| | octylcyclopropyl)heptadecan-8-amine |
| 43 | 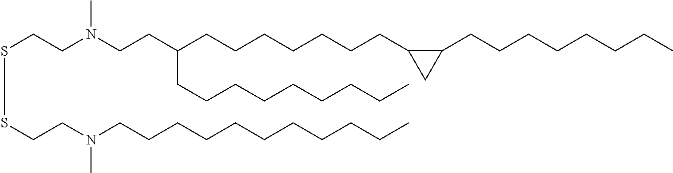 [SMILES: CCCCCCCCCCCN(C)CCSSCCN(C)CCC(CCCCCCCCC)CCCCCCCC1CC1CCCCCCCC] |
| | N-methyl-N-(2-((2-(methyl(undecyl)amino)ethyl)disulfaneyl)ethyl)-3-(7-(2- |
| | octylcyclopropyl)heptyl)dodecan-1-amine |
| 44 | 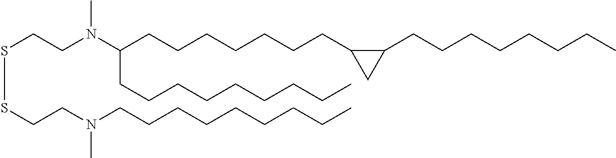 [SMILES: CCCCCCCCCC(CCCCCCCC1CC1CCCCCCCC)N(C)CCSSCCN(C)CCCCCCCCC] |
| | N-methyl-N-(2-((2-(methyl(nonyl)amino)ethyl)disulfaneyl)ethyl)-1-(2- |
| | octylcyclopropyl)heptadecan-8-amine |
| 45 | 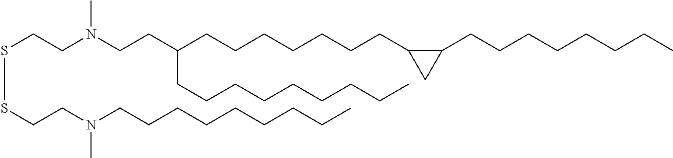 [SMILES: CCCCCCCCCC(CCCCCCCC1CC1CCCCCCCC)CCN(C)CCSSCCN(C)CCCCCCCCC] |
| | N-methyl-N-(2-((2-(methyl(nonyl)amino)ethyl)disulfaneyl)ethyl)-3-(7-(2- |
| | octylcyclopropyl)heptyl)dodecan-1-amine |
| 46 | 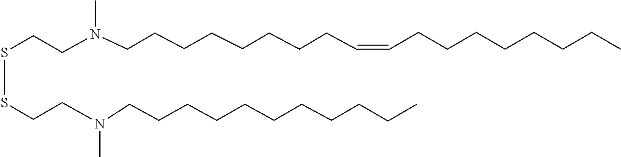 [SMILES: CCCCCCCC/C=C\CCCCCCCCN(C)CCSSCCN(C)CCCCCCCCCCC] |
| | (Z)-N-methyl-N-(2-((2-(methyl(undecyl)amino)ethyl)disulfaneyl)ethyl)octadec-9-en- |
| | 1-amine |
| 47 | 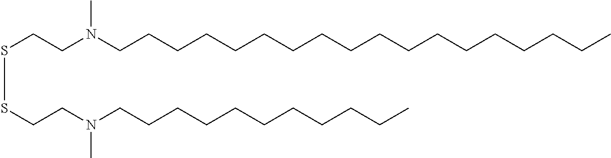 [SMILES: CCCCCCCCCCCCCCCCCCN(C)CCSSCCN(C)CCCCCCCCCCC] |
| | N-methyl-N-(2-((2-(methyl(undecyl)amino)ethyl)disulfaneyl)ethyl)octadecan-1- |
| | amine |
| 48 | 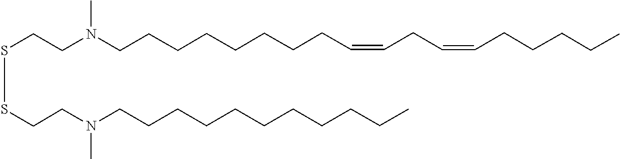 [SMILES: CCCCC/C=C\C/C=C\CCCCCCCCN(C)CCSSCCN(C)CCCCCCCCCCC] |
| | (9Z,12Z)-N-methyl-N-(2-((2- |
| | (methyl(undecyl)amino)ethyl)disulfaneyl)ethyl)octadeca-9,12-dien-1-amine |
| 49 | 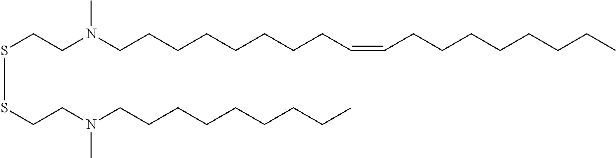 [SMILES: CCCCCCCC/C=C\CCCCCCCCN(C)CCSSCCN(C)CCCCCCCCC] |
| | (Z)-N-methyl-N-(2-((2-(methyl(nonyl)amino)ethyl)disulfaneyl)ethyl)octadec-9-en-1- |
| | amine |
| 50 | 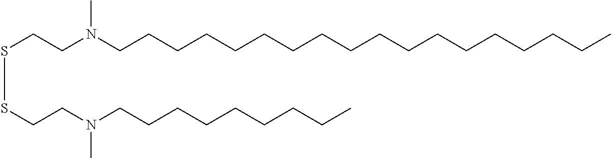 [SMILES: CCCCCCCCCCCCCCCCCCN(C)CCSSCCN(C)CCCCCCCCC] |
| | N-methyl-N-(2-((2-(methyl(nonyl)amino)ethyl)disulfaneyl)ethyl)octadecan-1-amine |
| 51 | 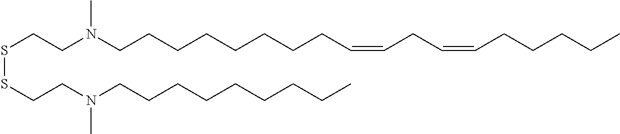 [SMILES: CCCCC/C=C\C/C=C\CCCCCCCCN(C)CCSSCCN(C)CCCCCCCCC] |
| | (9Z,12Z)-N-methyl-N-(2-((2-(methyl(nonyl)amino)ethyl)disulfaneyl)ethyl)octadeca- |
| | 9,12-dien-1-amine |

### Formula (II)

In some aspects, the ionizable lipids are of the Formula (II):

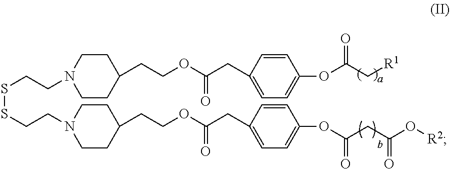

or a pharmaceutically acceptable salt thereof, wherein:
a is an integer ranging from 1 to 20 (e.g., a is 1, 2, 3, 4, 5, 6, 7, 8, 9, 10, 11, 12, 13, 14, 15, 16, 17, 18, 19, or 20);
b is an integer ranging from 2 to 10 (e.g., b is 2, 3, 4, 5, 6, 7, 8, 9, or 10);
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

In a second chemical embodiment, the ionizable lipid of the Formula (II) is of the Formula (XIII):

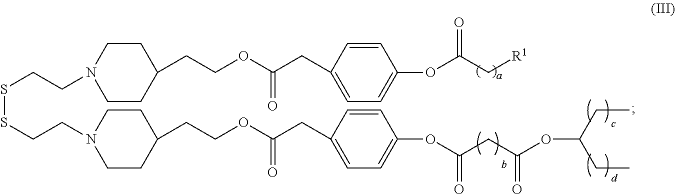

or a pharmaceutically acceptable salt thereof, wherein c and d are each independently integers ranging from 1 to 8 (e.g., 1, 2, 3, 4, 5, 6, 7, or 8), and wherein the remaining variables are as described for Formula (XII).

In a third chemical embodiment, c and d in the ionizable lipid of Formula (II) or (III) are each independently integers ranging from 2 to 8, 3 to 8, 3 to 7, 3 to 6, 3 to 5, 4 to 8, 4 to 7, 4 to 6, 5 to 8, 5 to 7, or 6 to 8, wherein the remaining variables are as described for Formula (XII).

In a fourth chemical embodiment, c in the ionizable lipid of Formula (II) or (III) is 2, 3, 4, 5, 6, 7, or 8, wherein the remaining variables are as described for Formula (XII) or the second or third chemical embodiment. Alternatively, as part of a fourth chemical embodiment, c and d in the ionizable lipid of Formula (XII) or (XIII) or a pharmaceutically acceptable salt thereof are each independently 1, 3, 5, or 7, wherein the remaining variables are as described for Formula (XII) or the second or third chemical embodiment.

In a fifth chemical embodiment, d in the ionizable lipid of Formula (II) or (III) is 2, 3, 4, 5, 6, 7, or 8, wherein the remaining variables are as described for Formula (II) or the second or third or fourth chemical embodiment. Alternatively, as part of a fourth chemical embodiment, at least one of c and d in the ionizable lipid of Formula (II) or (III) or a pharmaceutically acceptable salt thereof is 7, wherein the remaining variables are as described for Formula (II) or the second or third or fourth chemical embodiment.

In a sixth chemical embodiment, the ionizable lipid of Formula (II) or (III) is of the Formula (IV):

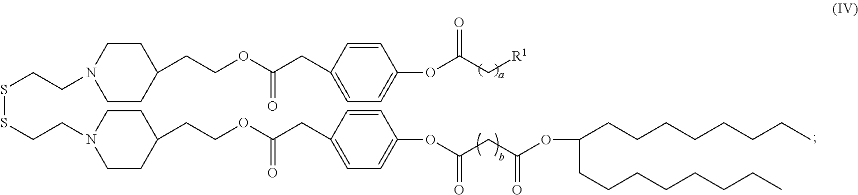

or a pharmaceutically acceptable salt thereof, wherein the remaining variables are as described for Formula (I).

In a seventh chemical embodiment, b in the ionizable lipid of Formula (II), (III), or (IV) is an integer ranging from 3 to 9, wherein the remaining variables are as described for Formula (II), or the second, third, fourth or fifth chemical embodiment. Alternatively, as part of a seventh chemical embodiment, b in the ionizable lipid of Formula (II), (III), or (IV) is an integer ranging from 3 to 8, 3 to 7, 3 to 6, 3 to 5, 4 to 9, 4 to 8, 4 to 7, 4 to 6, 5 to 9, 5 to 8, 5 to 7, 6 to 9, 6 to 8, or 7 to 9, wherein the remaining variables are as described for Formula (II), or the second, third, fourth or fifth chemical embodiment. In another alternative, as part of a seventh chemical embodiment, b in the ionizable lipid of Formula (II), (III), or (IV) is 3, 4, 5, 6, 7, 8, or 9, wherein the remaining variables are as described for Formula (XII), or the second, third, fourth or fifth chemical embodiment.

In an eighth chemical embodiment, a in the ionizable lipid of Formula (II), (III), or (IV) is an integer ranging from 2 to 18, wherein the remaining variables are as described for Formula (II), or the second, third, fourth, fifth, or seventh chemical embodiment. Alternatively, as part of an eighth embodiment, a in the ionizable lipid of Formula (II), (III), or (IV) is an integer ranging from 2 to 18, 2 to 17,2 to 16,2 to 15,2 to 14,2 to 13,2 to 12,2 to 11,2 to 10,2 to 9,2 to 8,2 to 7,2 to 6,2 to 5,2 to 4,3 to 18,3 to 17,3 to 16,3 to 15,3 to 14,3 to 13,3 to 12,3 to 11,3 to 10,3 to 9,3 to 8,3 to 7,3 to 6,3 to 5,4 to 18,4 to 17,4 to 16,4 to 15,4 to 14,4 to 13,4 to 12,4 to 11,4 to 10,4 to 9,4 to 8,4 to 7,4 to 6,5 to 18,5 to 17,5 to 16,5 to 15,5 to 14,5 to 13,5 to 12,5 to 11,5 to 10,5 to 9,25 to 8,5 to 7,6 to 18,6 to 17,6 to 16,6 to 15,6 to 14,6 to 13,6 to 12,6 to 11,6 to 10,6 to 9,6 to 8,7 to 18,7 to 17, 7 to 16, 7 to 15, 7 to 14, 7 to 13, 7 to 12, 7 to 11, 7 to 10, 7 to 9, 8 to 18, 8 to 17, 8 to 16, 8 to 15, 8 to 14,8 to 13,8 to 12,8 to 11,8 to 10, 9 to 18, 9 to 17, 9 to 16,9 to 15, 9 to 14, 9 to 13, 9 to 12, 9 to 11,10 to 18,1Oto 17,1Oto 16,1Oto 15,1Oto 14,1Oto 13,11 to 18,11 to 17,11 to 16,11 to 15,11 to 14, 11 to 13, 12 to 18, 12 to 17, 12 to 16, 12 to 15, 12 to 14,13 to 18,13 to 17,13 to 16,13 to 15, 14 to 18, 14 to 17, 14 to 16, 15 to 18, 15 to 17, or 16 to 18, wherein the remaining variables are as described for Formula (II), or the second, third, fourth, fifth, or seventh chemical embodiment. In another alternative, as part of an eighth embodiment, a in the ionizable lipid of Formula (II), (III), or (IV) is 2, 3, 4, 5, 6, 7, 8, 9, 10, 11, 12, 13, 14, 15, 16, 17, or 18, wherein the remaining variables are as described for Formula (II), or the second, third, fourth, fifth, or seventh chemical embodiment.

In a ninth chemical embodiment, R¹ in the ionizable lipid of Formula (II), (III), or (IV) or a pharmaceutically acceptable salt thereof is absent or is selected from (C₅-C₁₅)alkenyl, —C(O)O(C₄—Cis)alkyl, and cyclopropyl substituted with (C₄-C₁₆)alkyl, wherein the remaining variables are as described for Formula (II), (III), or (IV) or the second, third, fourth, fifth, seventh, or eighth chemical embodiment. Alternatively, as part of a ninth chemical embodiment, R¹ in the ionizable lipid of Formula (II), (III), or (IV) or a pharmaceutically acceptable salt thereof is absent or is selected from (C₅-C₁₅)alkenyl, —C(O)O(C₄-C₁₆)alkyl, and cyclopropyl substituted with (C₄-C₁₆)alkyl, wherein the remaining variables are as described for Formula (II), (III), or (IV) or the second, third, fourth, fifth, seventh, or eighth chemical embodiment. Alternatively, as part of a ninth chemical embodiment, R¹ in the ionizable lipid of Formula (II), (III), or (IV) or a pharmaceutically acceptable salt thereof is absent or is selected from (C₅-C₁₂)alkenyl, —C(O)O(C₄-C₁₂)alkyl, and cyclopropyl substituted with (C₄-C₁₂)alkyl, wherein the remaining variables are as described for Formula (II), (III), or (IV) or the second, third, fourth, fifth, seventh, or eighth chemical embodiment. In another alternative, as part of a ninth chemical embodiment, R¹ in the ionizable lipid of Formula (II), (III), or (IV) or a pharmaceutically acceptable salt thereof is absent or is selected from (C₅-C₁₀)alkenyl, —C(O)O(C₄—C₁₀)alkyl, and cyclopropyl substituted with (C₄-C₁₀)alkyl, wherein the remaining variables are as described for Formula (II), (III), or (IV) or the second, third, fourth, fifth, seventh, or eighth chemical embodiment.

In a tenth chemical embodiment, R¹ is C₁₀ alkenyl, wherein the remaining variables are as described in any one of the foregoing embodiments.

In an eleventh chemical embodiment, the alkyl in C(O)O(C₂-C₂₀)alkyl, —C(O)O(C₄-C₁₅)alkyl,

—C(O)O(C₄-C₁₂)alkyl, or —C(O)O(C₄-C₁₀)alkyl of R¹ in the ionizable lipid of Formula (II), (III), or (IV) or a pharmaceutically acceptable salt thereof is an unbranched alkyl, wherein the remaining variables are as described in any one of the foregoing embodiments. In one chemical embodiment, R¹ is —C(O)O(C₉ alkyl). Alternatively, in an eleventh chemical embodiment, the alkyl in —C(O)O(C₄-C₁₅)alkyl, —C(O)O(C₄-C₁₂)alkyl, or —C(O)O(C₄-C₁₀)alkyl of R¹ in the ionizable lipid of Formula (II), (III), or (IV) or a pharmaceutically acceptable salt thereof is a branched alkyl, wherein the remaining variables are as described in any one of the foregoing chemical embodiments. In one chemical embodiment, R¹ is

—C(O)O(C₁₇ alkyl), wherein the remaining variables are as described in any one of the foregoing chemical embodiments.

In a twelfth chemical embodiment, R¹ in the ionizable lipid of Formula (II), (III), or (IV) or a pharmaceutically acceptable salt thereof is selected from any group listed in Table 3 below, wherein the wavy bond in each of the groups indicates the point of attachment of the group to the rest of the lipid molecule, and wherein the remaining variables are as described for Formula (II), (III), or (IV) or the second, third, fourth, fifth, seventh, or eighth chemical embodiment. The present disclosure further contemplates the combination of any one of the R¹ groups in Table 4 with any one of the R² groups in Table 5, wherein the remaining variables are as described for Formula (II), (III), or (IV) or the second, third, fourth, fifth, seventh, or eighth chemical embodiment.

TABLE 3
Exemplary R¹ groups in Formula (II), (III), or (IV)

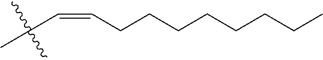 [SMILES: */C=C\CCCCCCCC]

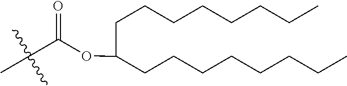 [SMILES: *C(=O)OC(CCCCCCCC)CCCCCCCC]

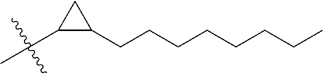 [SMILES: *C1CC1CCCCCCCC]

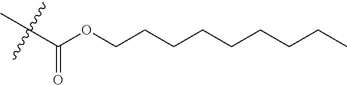 [SMILES: *C(=O)OCCCCCCCCC]

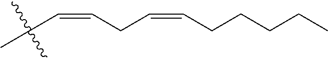 [SMILES: */C=C\C/C=C\CCCCC]

In a thirteenth chemical embodiment, R² in the ionizable lipid of Formula (II) or a pharmaceutically acceptable salt thereof is selected from any group listed in Table 4 below, wherein the wavy bond in each of the groups indicates the point of attachment of the group to the rest of the lipid molecule, and wherein the remaining variables are as described for Formula (II), or the seventh, eighth, ninth, tenth, or eleventh chemical embodiment.

TABLE 4
Exemplary R² groups in Formula (II)

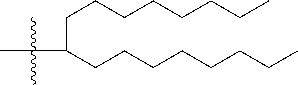 [SMILES: *C(CCCCCCCC)CCCCCCCC]

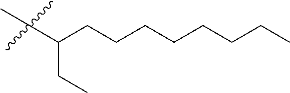 [SMILES: *C(CC)CCCCCCCC]

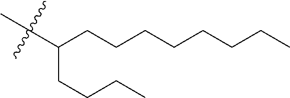 [SMILES: *C(CCCC)CCCCCCCC]

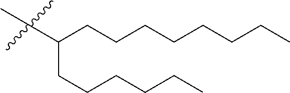 [SMILES: *C(CCCCCC)CCCCCCCC]

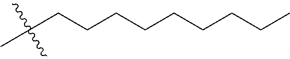 [SMILES: *CCCCCCCCC]

Specific examples are provided in Table 5 the exemplification section below and are included as part of a fourteenth chemical embodiment herein of ionizable lipids of Formula (II). Pharmaceutically acceptable salts as well as ionized and neutral forms are also included.

TABLE 5
Exemplary ionizable lipids of Formula (II), (III), or (IV)

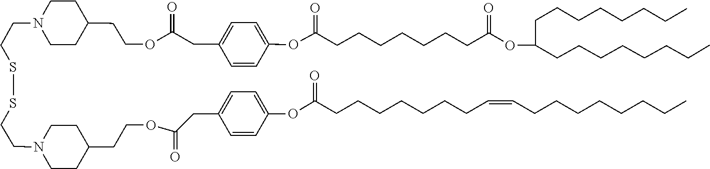 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 52
1-(heptadecan-9-yl) 9-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) nonanedioate

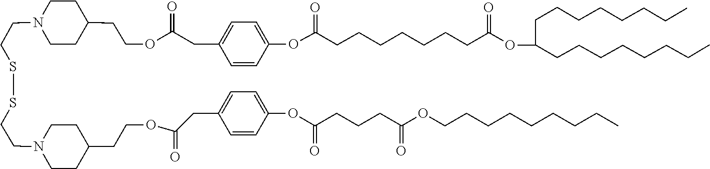 [SMILES: CCCCCCCCCOC(=O)CCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 53
1-(heptadecan-9-y1) 9-(4-(2-(2-(1-(2-((2-(4-(2-(2-(4-((5-(nonyloxy)-5-
oxopentanoyl)oxy)phenyl)acetoxy)ethyl) piperidin-1-yl)ethyl)disulfaneyl)ethyl) piperidin-4-
yl)ethoxy)-2-oxoethyl)phenyl) nonanedioate

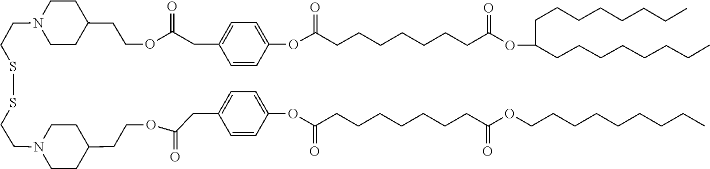 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 54
1-(heptadecan-9-yl) 9-(4-(2-(2-(1-(2-((2-(4-(2-(2-(4-((9-(nonyloxy)-9-
oxononanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

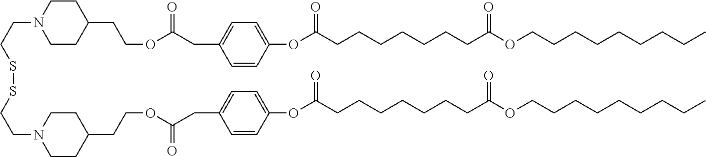 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCCCCCCCC)cc4)CC3)CC2)cc1]

Lipid 55
1-(heptadecan-9-y1) 9-(4-(2-(2-(1-(2-((2-(4-(2-(2-(4-((5-(nonyloxy)-5-
oxopentanoyl)oxy)phenyl)acetoxy)ethyl)
piperidin-1-yl)ethyl)disulfaneyl)ethyl)piperidin-4-yl)ethoxy)-2-oxoethyl)phenyl) nonanedioate

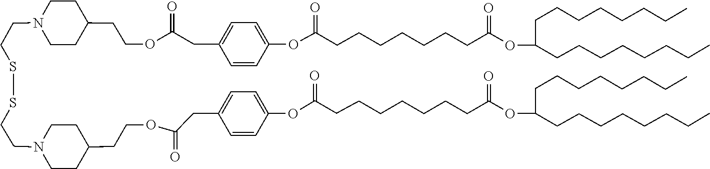 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 56
O′1,O1-((((((disulfanediylbis(ethane-2,1-diyl))bis(piperidine-1,4-diyl))bis(ethane-2,1-
diyl))bis(oxy))bis(2-oxoethane-2,1-diyl))bis(4,1-phenylene)) 9,9'-di(heptadecan-9-yl)
di(nonanedioate)

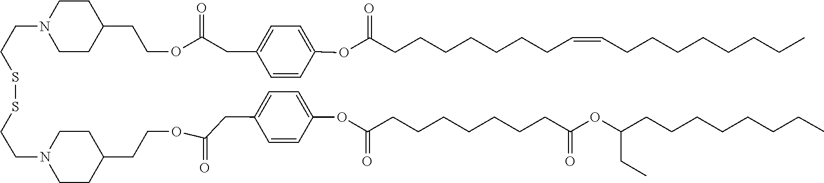 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

Lipid 57
1-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) 9-(undecan-3-yl)
nonanedioate

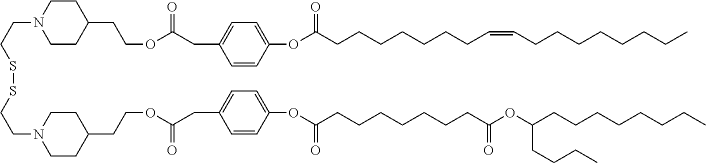 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

Lipid 58
1-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) 9-(tridecan-5-y1)
nonanedioate

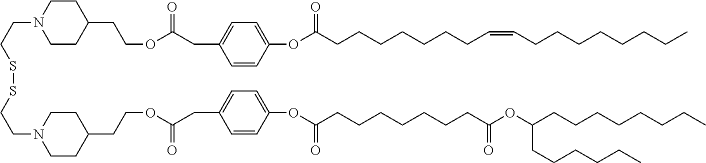 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

Lipid 59
1-(4-(2-(2-(1-(2-((2-(4-(2-(2-(4-(oleoyloxy)phenyl)acetoxy)ethyl)piperidin-1-
yl)ethyl)disulfaneyl)ethyl)piperidin-4-yl)ethoxy)-2-oxoethyl)phenyl) 9-(pentadecan-7-y1)
nonanedioate

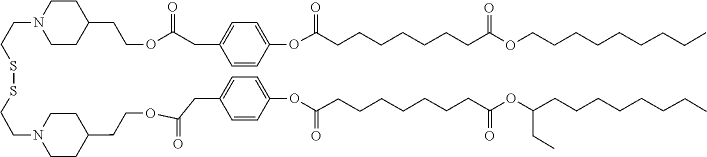 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

Lipid 60
1-nonyl 9-(4-(2-oxo-2-(2-(1-(2-((2-(4-(2-(2-(4-((9-oxo-9-(undecan-3-
yloxy)nonanoyl)oxy)phenyl)acetoxy)ethyl)piperidin-1-yl)ethyl)disulfaneyl)ethyl)piperidin-4-
yl)ethoxy)ethyl)phenyl) nonanedioate

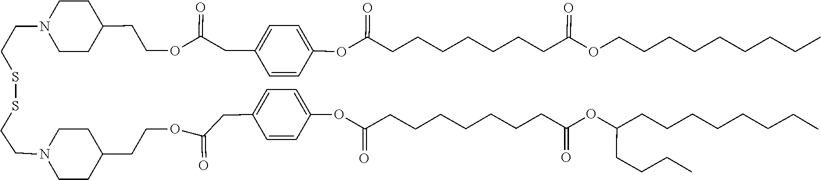 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

Lipid 61
1-nonyl 9-(4-(2-oxo-2-(2-(1-(2-((2-(4-(2-(2-(4-((9-oxo-9-(tridecan-5-
yloxy)nonanoyl)oxy)phenyl)acetoxy)ethyl)piperidin-1-yl)ethyl)disulfaneyl)ethyl)piperidin-4-
yl)ethoxy)ethyl)phenyl) nonanedioate

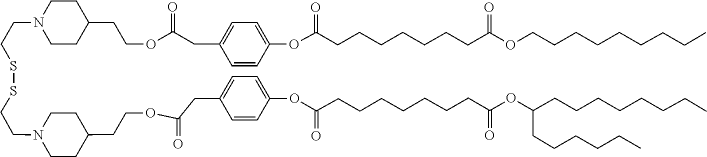 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

Lipid 62
1-nonyl 9-(4-(2-oxo-2-(2-(1-(2-((2-(4-(2-(2-(4-((9-oxo-9-(pentadecan-7-
yloxy)nonanoyl)oxy)phenyl)acetoxy)ethyl)piperidin-1-yl)ethyl)disulfaneyl)ethyl)piperidin-4-
yl)ethoxy)ethyl)phenyl) nonanedioate

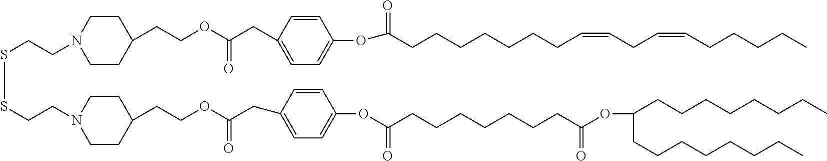 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 63
1-(heptadecan-9-y1) 9-(4-(2-(2-(1-(2-((2-(4-(2-(2-(4-(((9Z,12Z)-octadeca-9,12-
dienoyl)oxy)phenyl)acetoxy)ethyl)piperidin-1-yl)ethyl)disulfaneyl)ethyl)piperidin-4-yl)ethoxy)-2-
oxoethyl)phenyl) nonanedioate

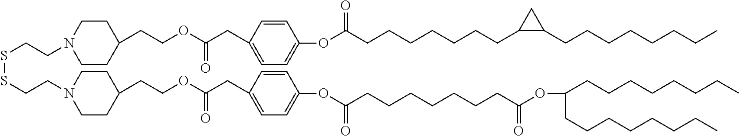 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC5CC5CCCCCCCC)cc4)CC3)CC2)cc1]

Lipid 64
1-(heptadecan-9-y1) 9-(4-(2-(2-(1-(2-((2-(4-(2-(2-(4-((8-(2-
octylcyclopropyl)octanoyl)oxy)phenyl)acetoxy)ethyl)piperidin-1-
yl)ethyl)disulfaneyl)ethyl)piperidin-4-yl)ethoxy)-2-oxoethyl)phenyl) nonanedioate

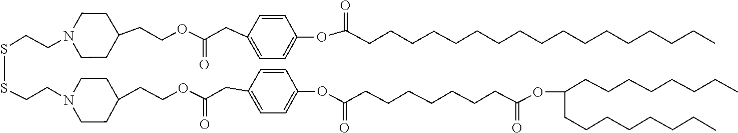 [SMILES: CCCCCCCCCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 65
1-(heptadecan-9-yl) 9-(4-(2-oxo-2-(2-(1-(2-((2-(4-(2-(2-(4-
(stearoyloxy)phenyl)acetoxy)ethyl)piperidin-1-yl)ethyl)disulfaneyl)ethyl)piperidin-4-
yl)ethoxy)ethyl)phenyl) nonanedioate

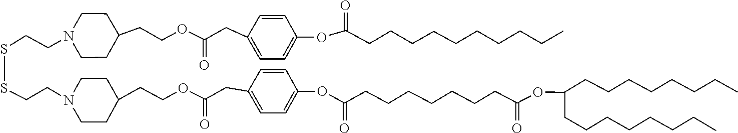 [SMILES: CCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 66
1-(heptadecan-9-yl) 9-(4-(2-oxo-2-(2-(1-(2-((2-(4-(2-(2-(4-
(undecanoyloxy)phenyl)acetoxy)ethyl)piperidin-1-yl)ethyl)disulfaneyl)ethyl)piperidin-4-
yl)ethoxy)ethyl)phenyl) nonanedioate

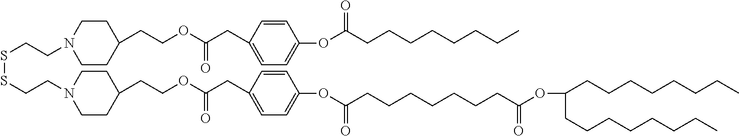 [SMILES: CCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

Lipid 67
1-(heptadecan-9-yl) 9-(4-(2-(2-(1-(2-((2-(4-(2-(2-(4-(nonanoyloxy)phenyl)acetoxy)ethyl)piperidin-
1-yl)ethyl)disulfaneyl)ethyl)piperidin-4-yl)ethoxy)-2-oxoethyl)phenyl) nonanedioate

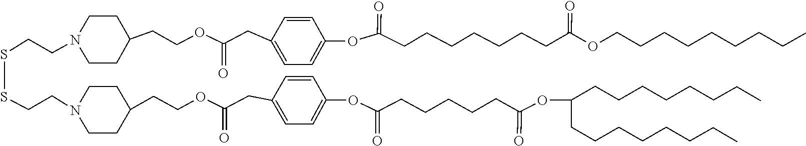 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]

Lipid 68
1-nonyl 9-(4-(2-(2-(1-(2-((2-(4-(2-(2-(4-((9-((3-octylundecyl)oxy)-9-
oxononanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

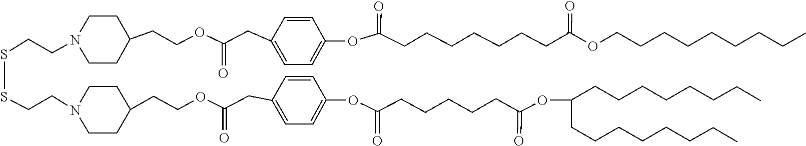 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]

Lipid 69
1-(4-(2-(2-(1-(2-((2-(4-(2-(2-(4-((7-(heptadecan-9-yloxy)-7-
oxoheptanoyl)oxy)phenyl)acetoxy)ethyl)piperidin-1-yl)ethyl)disulfaneyl)ethyl)piperidin-4-
yl)ethoxy)-2-oxoethyl)phenyl) 9-nonyl nonanedioate

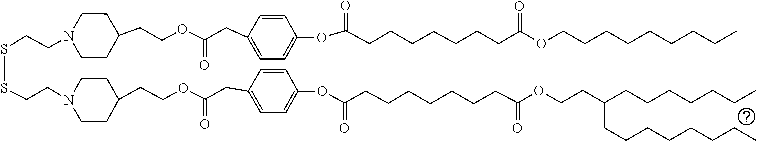 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCC(CCCCCCC)CCCCCCCC)cc4)CC3)CC2)cc1]

Lipid 70
1-nonyl 9-(4-(2-(2-(1-(2-((2-(4-(2-(2-(4-((9-((3-octylundecyl)oxy)-9-
oxononanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

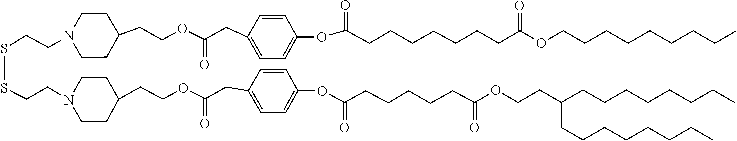 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OCCC(CCCCCCCC)CCCCCCCC)cc4)CC3)CC2)cc1]

Lipid 71
1-nonyl 9-(4-(2-(2-(1-(2-((2-(4-(2-(2-(4-((7-((3-octylundecyl)oxy)-7-
oxoheptanoyl)oxy)phenyl)acetoxy)ethyl)piperidin-1-yl)ethyl)disulfaneyl)ethyl)piperidin-4-
yl)ethoxy)-2-oxoethyl)phenyl) nonanedioate

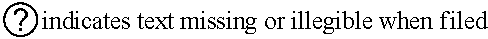

In some embodiments, a lipid nanoparticle of the present disclosure comprises 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(tridecan-5-yl) nonanedioate, listed above as Lipid 58:

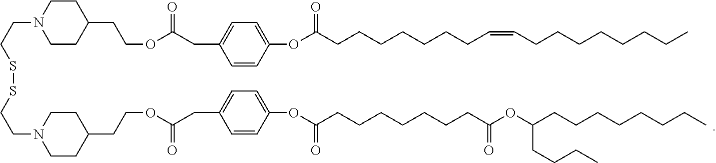 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

### Formula (V)

In some aspects, the ionizable lipids are of the Formula (V):

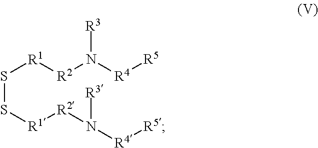

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

In a second chemical aspect, R¹ and R″ in the ionizable lipids of the Formula (V) each independently (C₁-C₆)alkylene, wherein the remaining variables are as described above for Formula (V). Alternatively, as part of a second chemical aspect, R¹ and R″ in the ionizable lipids of the Formula (V) each independently (C₁-C₃)alkylene, wherein the remaining variables are as described above for Formula (V).

In a third chemical aspect, the ionizable lipids of the Formula (V) are of the Formula (VI):

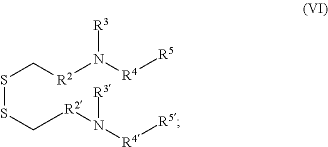

or a pharmaceutically acceptable salt thereof, wherein the remaining variables are as described above for Formula (V).

In a fourth chemical aspect, the ionizable lipids of the Formula (V) are of the Formula (VII) or (VIII):

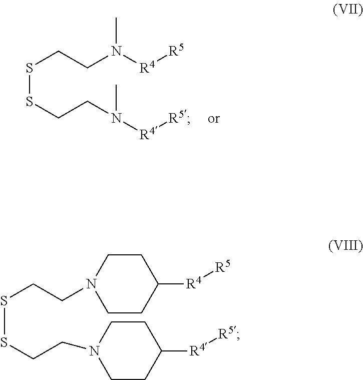

or a pharmaceutically acceptable salt thereof, wherein the remaining variables are as described above for Formula (V).

In a fifth chemical aspect, the ionizable lipids of the Formula (V) are of the Formula (IX) or

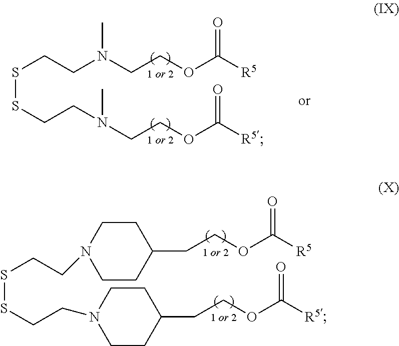

or a pharmaceutically acceptable salt thereof, wherein the remaining variables are as described above for Formula (V).

In a sixth chemical aspect, the ionizable lipids of the Formula (V) are of the Formula (XI), (XII), (XIII), or (XIV):

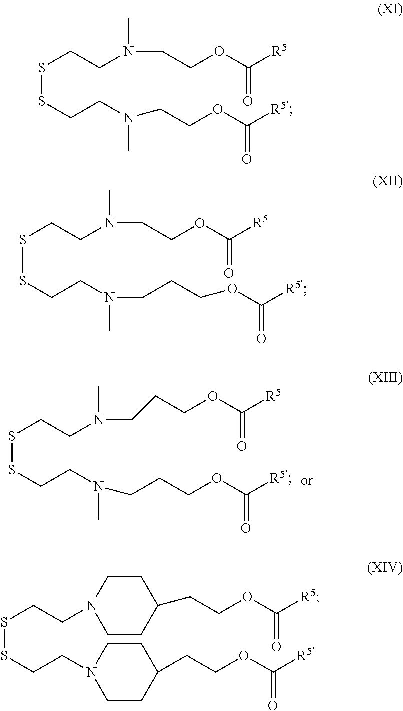

or a pharmaceutically acceptable salt thereof, wherein the remaining variables are as described above for Formula (XV).

In a seventh chemical aspect, at least one of R⁵ and R″ in the ionizable lipid of Formula (V), (VI), (VII), (VIII), (IX), (X), (XI), (XII), (XIII), or (XIV) is a branched alkyl or branched alkenyl (number of carbon atoms as described above for Formula (V), (VI), (VII), (VIII), (IX), (X), (XI), (XII), (XIII), or (XIV)). In another alternative, as part of a seventh chemical aspect, one of R⁵ and R^{5′} in the ionizable lipid of Formula (V), (VI), (VII), (VIII), (IX), (X), (XI), (XII), (XIII), or (XIV) is a branched alkyl or branched alkenyl. In another alternative, as part of a seventh chemical aspect, R in the ionizable lipid of Formula (V), (VI), (VII), (VIII), (IX), (X), (XI), (XII), (XIII), or (XIV) is a branched alkyl or branched alkenyl. In another alternative, as part of a seventh chemical aspect, R^{5′} in the ionizable lipid of Formula (V), (VI), (VII), (VIII), (IX), (X), (XI), (XII), (XIII), or (XIV) is a branched alkyl or branched alkenyl.

In an eighth chemical aspect, R⁵ in the ionizable lipid of Formula (V), (VI), (VII), (VIII), (IX), (X), (XI), (XII), (XIII), or (XIV) is a (C₆-C₂₆)alkyl or (C₆-C₂₆)alkenyl, each of which are optionally interrupted with —C(O)O— or (C₃-C₆)cycloalkyl, wherein the remaining variables are as described above for Formula (I). Alternatively, as part of a seventh chemical aspect, R⁵ in the ionizable lipid of Formula (V), (VI), (VII), (VIII), (IX), (X), (XI), (XII), (XIII), or (XIV) is a (C₆-C₂₆)alkyl or (C₆-C₂₆)alkenyl, each of which are optionally interrupted with —C(O)O— or (C₃—C₅)cycloalkyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₇-C₂₆)alkyl or (C₇-C₂₆)alkenyl, each of which are optionally interrupted with —C(O)O— or (C₃-C₅)cycloalkyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₅-C₂₆)alkyl or (C₅-C₂₆)alkenyl, each of which are optionally interrupted with —C(O)O— or (C₃—C₅)cycloalkyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₆-C₂₄)alkyl or (C₆-C₂₄)alkenyl, each of which are optionally interrupted with —C(O)O— or cyclopropyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₅-C₂₄)alkyl or (C₅-C₂₄)alkenyl, wherein said (C₅-C₂₄)alkyl is optionally interrupted with —C(O)O— or cyclopropyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₅-C₁₀)alkyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₁₄-C₁₆)alkyl interrupted with cyclopropyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₁₀-C₂₄)alkyl interrupted with —C(O)O—, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is a (C₁₆-C₁₅)alkenyl, wherein the remaining variables are as described above for Formula (V). In another alternative, as part of an eighth chemical aspect, R⁵ in the ionizable lipid of Formula (V), (VI), (VII), (VIII), (IX), (X), (XI), (XII), (XIII), or (XIV) is —(CH₂)₃C(O)O(CH₂)₈CH₃, —(CH₂)₅C(O)O(CH₂)₈CH₃, —(CH₂)₇C(O)O(CH₂)₈CH₃, —(CH₂)₇C(O)OCH[(CH₂)₇CH₃]₂, —(CH₂)₇—C₃H₆—(CH₂)₇CH₃, —(CH₂)₇CH₃, —(CH₂)₉CH₃, —(CH₂)₁₆CH₃, —(CH₂)₇CH═CH(CH₂)₇CH₃, or —(CH₂)₇CH═CHCH₂CH═CH(CH₂)₄CH₃, wherein the remaining variables are as described above for Formula (XV).

In a ninth chemical aspect, R^{5′} in the ionizable lipid of Formula (V), (VI), (VII), (VIII), (IX), (X), (XI), (XII), (XIII), or (XIV) is a (C₁₅-C_{2M})alkyl interrupted with —C(O)O—, wherein the remaining variables are as described above for Formula (V) or the eighth chemical aspect. Alternatively, as part of a ninth chemical aspect, R^{5′} in the ionizable lipid of Formula (V), (VI), (VII), (VIII), (IX), (X), (XI), (XII), (XIII), or (XIV) is a (C₁₇-C₂s)alkyl interrupted with —C(O)O—, wherein the remaining variables are as described above for Formula (V) or the eighth chemical aspect. In another alternative, as part of a ninth embodiment, R^{5′} in the ionizable lipid of Formula (V), (VI), (VII), (VIII), (IX), (X), (XI), (XII), (XIII), or (XIV) is a (C₁₉-C_{2M})alkyl interrupted with —C(O)O—, wherein the remaining variables are as described above for Formula (V) or the eighth chemical aspect. In another alternative, as part of a ninth chemical aspect, R^{5′} in the ionizable lipid of Formula (V), (VI), (VII), (VIII), (IX), (X), (XI), (XII), (XIII), or (XIV) is a (C₁₇-C₂₆)alkyl interrupted with —C(O)O—, wherein the remaining variables are as described above for Formula (V) or the eighth chemical aspect. In another alternative, as part of a ninth embodiment, R^{5′} in the ionizable lipid of Formula (V), (VI), (VII), (VIII), (IX), (X), (XI), (XII), (XIII), or (XIV) is a (C₁₉-C₂₆)alkyl interrupted with —C(O)O—, wherein the remaining variables are as described above for Formula (V) or the eighth chemical aspect. In another alternative, as part of a ninth chemical aspect, R^{5′} in the ionizable lipid of Formula (V), (VI), (VII), (VIII), (IX), (X), (XI), (XII), (XIII), or (XIV) is a (C₂o-C₂₆)alkyl interrupted with —C(O)O—, wherein the remaining variables are as described above for Formula (V) or the eighth chemical aspect. In another alternative, as part of a ninth embodiment, R^{5′} is a (C₂₂-C₂₄)alkyl interrupted with —C(O)O—, wherein the remaining variables are as described above for Formula (V) or the eighth chemical aspect. In another alternative, as part of a ninth embodiment, R^{5′} is —(CH₂)₅C(O)OCH[(CH₂)₇CH₃]₂, —(CH₂)₇C(O)OCH[(CH₂)₇CH₃]₂, —(CH₂)₅C(O)OCH(CH₂)₂[(CH₂)₇CH₃]₂, or —(CH₂)₇C(O)OCH(CH₂)₂[(CH₂)₇CH₃]₂, wherein the remaining variables are as described above for Formula (V) or the eighth chemical aspect.

In another aspect, the ionizable lipid of Formula (V), (VI), (VIII), (VIII), (IX), (X), (XII), (XIII), or (XIV) may be selected from any of the following lipids in Table 6 or a pharmaceutically acceptable salt thereof.

TABLE 6
Exemplary ionizable lipids of Formula (V), (VI), (VIII), (VIII), (IX), (X), (XII), (XIII), or (XIV)

| Lipid | |
|---|---|
| No. | Lipid Structure and Name |
| 72 | 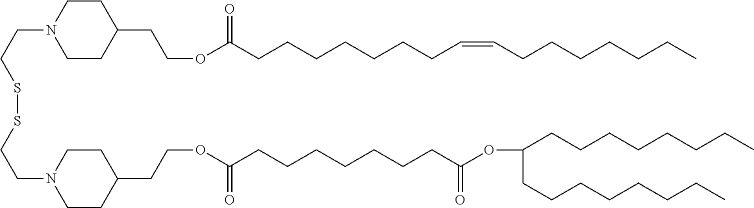 [SMILES: CCCCCCC/C=C\CCCCCCCC(=O)OCCC1CCN(CCSSCCN2CCC(CCOC(=O)CCCCCCCC(=O)OC(CCCCCCCC)CCCCCCCC)CC2)CC1] |
| | (Z)-1-(2-(1-(2-((2-(4-(2-(heptadec-9-enoyloxy)ethyl)piperidin-1- |
| | yl)ethyl)disulfaneyl)ethyl)piperidin-4-yl)ethyl) 9-(heptadecan-9-y1) |
| | nonanedioate |
| 73 | 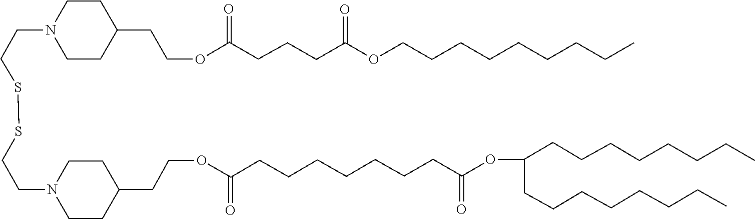 |
| | 1-(heptadecan-9-y1) 9-(2-(1-(2-((2-(4-(2-((5-(nonyloxy)-5- |
| | oxopentanoyl)oxy)ethyl)piperidin-1-yl)ethyl)disulfaneyl)ethyl)piperidin-4- |
| | yl)ethyl) nonanedioate |
| 74 | 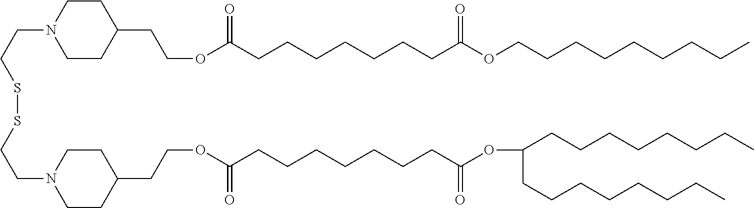 [SMILES: CCCCCCCCCOC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| | 1-(heptadecan-9-yl) 9-(2-(1-(2-((2-(4-(2-((9-(nonyloxy)-9- |
| | oxononanoyl)oxy)ethyl)piperidin-1-yl)ethyl)disulfaneyl)ethyl)piperidin-4- |
| | yl)ethyl) nonanedioate |
| 75 | 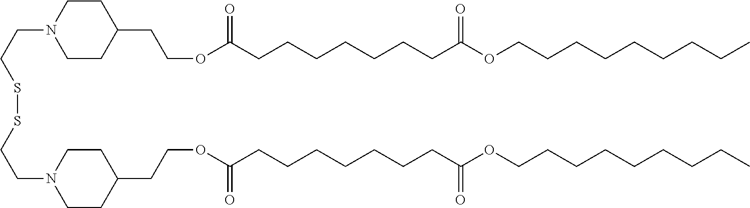 [SMILES: CCCCCCCCCOC(=O)CCCCCCCC(=O)OCCC1CCN(CCSSCCN2CCC(CCOC(=O)CCCCCCCC(=O)OCCCCCCCCC)CC2)CC1] |
| | O′1,O1-(((disulfanediylbis(ethane-2,1-diyl))bis(piperidine-1,4- |
| | diyl))bis(ethane-2,1-diyl)) 9,9′-dinonyl di(nonanedioate) |
| 76 | 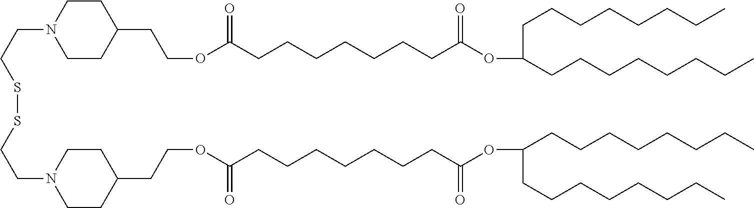 [SMILES: CCCCCCCCC(CCCCCCCC)OC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| | O′1,O1-(((disulfanediylbis(ethane-2,1-diyl))bis(piperidine-1,4- |
| | diyl))bis(ethane-2,1-diyl)) 9,9′-di(heptadecan-9-yl) di(nonanedioate) |

### Formula (XV)

In some aspects, the ionizable lipids are of the Formula (XV):

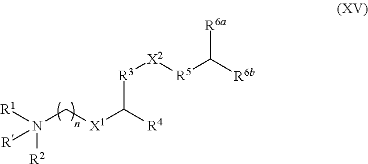

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₆ unbranched alkyl, C₂-C₁₆ unbranched alkenyl, or

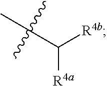

wherein:
R^{4a} and R^{4b} are each independently C₁-C₁₆ unbranched alkyl or C₂-C₁₆ unbranched alkenyl;
R⁵ is absent, C₁-C₅ alkylene, or C₂-C₅ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6.

In a second embodiment, in the ionizable lipid according to the first embodiment, or a pharmaceutically acceptable salt thereof, X¹ and X² are the same; and all other remaining variables are as described for Formula (V) or the first embodiment.

In a third embodiment, in the ionizable lipid according to the first or second embodiment, or a pharmaceutically acceptable salt thereof, X¹ and X² are each independently —OC(═O)—, —SC(═O)—, —OC(═S)—, —C(═O)O—, —C(═O)S—, or —S—S—; or X¹ and X² are each independently —C(═O)O—, —C(═O)S—, or —S—S—; or X¹ and X² are each independently —C(═O)O- or —S—S—; and all other remaining variables are as described for Formula V or any one of the preceding embodiments.

In a fourth embodiment, the ionizable lipid of the present disclosure is represented by Formula (XVI):

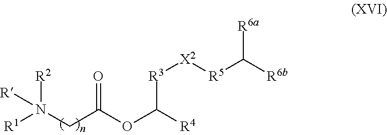

or a pharmaceutically acceptable salt thereof, wherein n is an integer selected from 1, 2, 3, and 4; and all other remaining variables are as described for Formula (XV) or any one of the preceding embodiments.

In a fifth embodiment, the ionizable lipid of the present disclosure is represented by Formula (XVII):

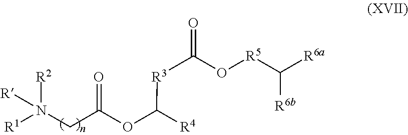

or a pharmaceutically acceptable salt thereof, wherein n is an integer selected from 1, 2, and 3; and all other remaining variables are as described for Formula (XV), Formula (XVI) or any one of the preceding embodiments.

In a sixth embodiment, the ionizable lipid of the present disclosure is represented by Formula (XVIII):

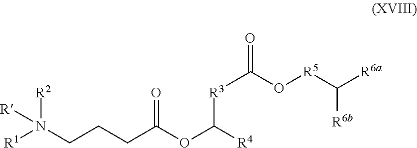

or a pharmaceutically acceptable salt thereof, and all other remaining variables are as described for Formula (XV), Formula (XVI), Formula (XVII) or any one of the preceding embodiments.

In a seventh embodiment, in the ionizable lipid according to Formula (XV), Formula (XVI), Formula (XVII), Formula (XVIII) or any one of the preceding embodiments, or a pharmaceutically acceptable salt thereof, R¹ and R²are each independently hydrogen, C₁-C₆ alkyl or C₂-C₆ alkenyl, or C₁-C₅ alkyl or C₂-C₅ alkenyl, or C₁-C₄ alkyl or C₂-C₄ alkenyl, or C₆ alkyl, or C₅ alkyl, or C₄ alkyl, or C₃ alkyl, or C₂ alkyl, or C₁ alkyl, or C₆ alkenyl, or C₅ alkenyl, or C₄ alkenyl, or C₃ alkenyl, or C₂ alkenyl; and all other remaining variables are as described for Formula (XV), Formula (XVI), Formula (XVII), Formula (XVIII) or any one of the preceding embodiments.

In an eighth embodiment, the ionizable lipid of the present disclosure is represented by Formula (XIX):

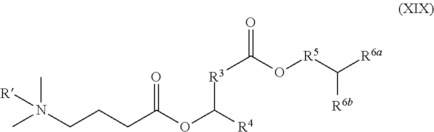

or a pharmaceutically acceptable salt thereof, and all other remaining variables are as described for Formula (XV), Formula (XVI), Formula (XVII), Formula (XVIII) or any one of the preceding embodiments.

In a ninth embodiment, in the ionizable lipid according to Formula (XV), Formula (XVI), Formula (XVII), Formula (XVIII), Formula (XIX) or any one of the preceding embodiments, or a pharmaceutically acceptable salt thereof, R³ is C₁-C₉ alkylene or C₂-C₉ alkenylene, C₁-C₇ alkylene or C₂-C₇ alkenylene, C₁-C₅ alkylene or C₂-C₅ alkenylene, or C₂-C₅ alkylene or C₂-C₅ alkenylene, or C₃-C₇, alkylene or C₃-C₇ alkenylene, or C₅-C₇ alkylene or C₅-C₇ alkenylene; or R³ is C₁₂ alkylene, Cu alkylene, C₁₀ alkylene, C₉ alkylene, or C₅ alkylene, or C₇ alkylene, or C₆ alkylene, or C₅ alkylene, or C₄ alkylene, or C₃ alkylene, or C₂ alkylene, or C₁ alkylene, or C₁₂ alkenylene, C₁₁ alkenylene, C₁₀ alkenylene, C₉ alkenylene, or C₅ alkenylene, or C₇ alkenylene, or C₆ alkenylene, or C₅ alkenylene, or C₄ alkenylene, or C₃ alkenylene, or C₂ alkenylene; and all other remaining variables are as described for Formula (XV), Formula (XVI), Formula (XVII), Formula (XVIII), Formula (XIX) or any one of the preceding embodiments.

In a tenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₅ alkylene, C₇ alkylene, C₆ alkylene, C₅ alkylene, C₄ alkylene, C₃ alkylene, C₂ alkylene, C₁ alkylene, C₅ alkenylene, C₇ alkenylene, C₆ alkenylene, C₅ alkenylene, C₄ alkenylene, C₃ alkenylene, or C₂ alkenylene; and all other remaining variables are as described for Formula (XV), Formula (XVI), Formula (XVII), Formula (XVIII), Formula (XIX) or any one of the preceding embodiments.

In an eleventh embodiment, in the cationic lipid according to Formula (XV), Formula (XVI), Formula (XVII), Formula (XVIII), Formula (XIX) or any one of the preceding embodiments, or a pharmaceutically acceptable salt thereof, R⁴ is C₁-C₁₄ unbranched alkyl, C₂-C₁₄ unbranched alkenyl, or

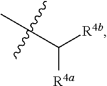

R^{4a}, wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₇ unbranched alkyl or C₅-C₇ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₅ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₅ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

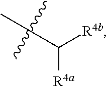

wherein R^{4a} and R^{4b} are each independently C₂-C₁₀ unbranched alkyl or C₂-C₁o unbranched alkenyl; or R⁴ is

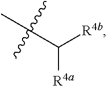

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₅ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁ unbranched alkenyl, C₁₀ unbranched alkenyl, C₉ unbranched alkenyl, C₅ unbranched alkenyl, C₇ unbranched alkenyl, C₆ unbranched alkenyl, C₅ unbranched alkenyl, C₄ unbranched alkenyl, C₃ unbranched alkenyl, or C₂alkenyl; and all other remaining variables are as described for Formula (XV), Formula (XVI), Formula (XVII), Formula (XVIII), Formula (XIX) or any one of the preceding embodiments.

In a twelfth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} are each independently C₆-C₁₄ alkyl or C₆-C₁₄ alkenyl; or R^{6a} and R^{6b} are each independently C₈-C₁₂ alkyl or C₈-C₁₂ alkenyl; or R^{6a} and R^{6b} are each independently C₁₆ alkyl, C₁₅ alkyl, C₁₄ alkyl, C₁₃ alkyl, C₁₂ alkyl, C₁ alkyl, C₁₀ alkyl, C₉ alkyl, C₈ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₅ alkenyl, or C₇ alkenyl; provided that the total number of carbon atoms in R^{6a} and R^{6b} as combined is greater than 15; and all other remaining variables are as described for Formula (XV), Formula (XVI), Formula (XVII), Formula (XVIII), Formula (XIX) or any one of the preceding embodiments.

In a thirteenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} contain an equal number of carbon atoms with each other; or R^{6a} and R^{6b} are the same; or R^{6a} and R^{6b} are both C₁₆ alkyl, C₁₅ alkyl, C₁₄ alkyl, C₁₃ alkyl, C₁₂ alkyl, C₁₁ alkyl, C₁₀ alkyl, C₉ alkyl, C₅ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₅ alkenyl, or C₇ alkenyl; provided that the total number of carbon atoms in R^{6a} and R^{6b} as combined is greater than 15; and all other remaining variables are as described for Formula (XV), Formula (XVI), Formula (XVII), Formula (XVIII), Formula (XIX) or any one of the preceding embodiments.

In a fourteenth embodiment, in the cationic lipid according to Formula (XV), Formula (XVI), Formula (XVII), Formula (XVIII), Formula (XIX) or any one of the preceding embodiments, or a pharmaceutically acceptable salt thereof, R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and Rib differs by one carbon atom; or R^{6a} is C₇ alkyl and R^{6a} is C₈ alkyl, R^{6a} is C₅ alkyl and R^{6a} is C₇ alkyl, R^{6a} is C₈ alkyl and R^{6a} is C₉ alkyl, R^{6a} is C₉ alkyl and R^{6a} is C₅ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₅ alkyl and R^{6a} is C₁₀ alkyl, R^{6a} is C₁₀ alkyl and R^{6a} is C₅ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, R^{6a} is C₁₁ alkyl and R^{6a} is C₁₃ alkyl, or R^{6a} is C₁₃ alkyl and R^{6a} is C₁₁ alkyl, etc.; and all other remaining variables are as described for Formula I, Formula II, Formula III, Formula IV, Formula V or any one of the preceding embodiments.

In one embodiment, the cationic lipid of the present disclosure or the cationic lipid of Formula (XV), Formula (XVI), Formula (XVII), Formula (XVIII), or Formula (XIX) is any one lipid selected from the lipids in Table 7 or a pharmaceutically acceptable salt thereof:

TABLE 7
Exemplary lipids of the present disclosure, including exemplary lipids of Formula (XV),
Formula (XVI), Formula (XVII), Formula (XVIII), Formula (XIX)

| Lipid | |
|---|---|
| No. | Lipid Structure and Name |
| 77 | 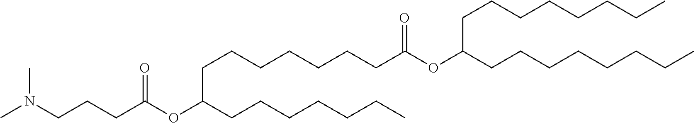 [SMILES: CCCCCCCCC(CCCCCCCC)OC(=O)CCCCCCCC(CCCCCCC)OC(=O)CCCN(C)C] |
| | henicosan-11-yl 9-((4-(dimethylamino)butanoyl)oxy)hexadecanoate |
| 78 | 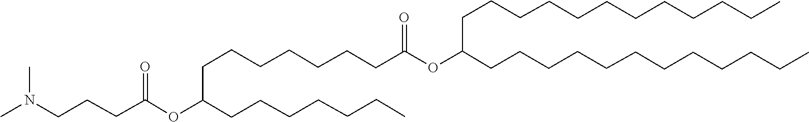 [SMILES: CCCCCCCCCCCCC(CCCCCCCCCCCC)OC(=O)CCCCCCCC(CCCCCCC)OC(=O)CCCN(C)C] |
| | pentacosan-13-yl 9-((4-(dimethylamino)butanoyl)oxy)hexadecanoate |
| 79 | 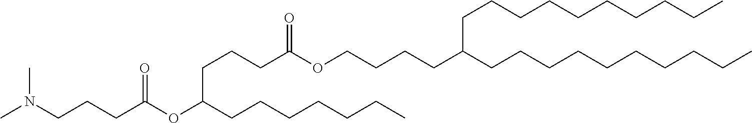 [SMILES: CCCCCCCCCCC(CCCCCCCCCC)CCCCOC(=O)CCCC(CCCCCCC)OC(=O)CCCN(C)C] |
| | 5-decylpentadecyl 5-((4-(dimethylamino)butanoyl)oxy)dodecanoate |
| 80 | 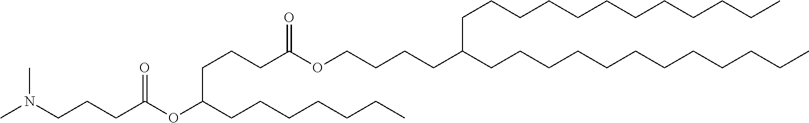 [SMILES: CCCCCCCCCCCCC(CCCCCCCCCCCC)CCCCOC(=O)CCCC(CCCCCCC)OC(=O)CCCN(C)C] |
| | 5-dodecylheptadecyl 5-((4-(dimethylamino)butanoyl)oxy)dodecanoate |
| 81 | 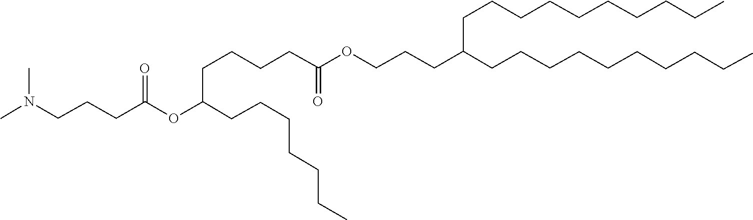 [SMILES: CCCCCCCCCCC(CCCCCCCCCC)CCCOC(=O)CCCCC(CCCCCCC)OC(=O)CCCN(C)C] |
| | 4-decyltetradecyl 6-((4-(dimethylamino)butanoyl)oxy)tridecanoate |
| 82 | 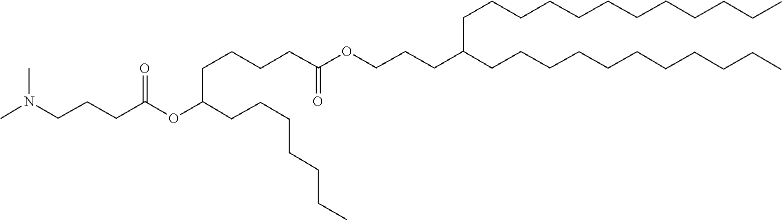 [SMILES: CCCCCCCCCCCCC(CCCCCCCCCCC)CCCOC(=O)CCCCC(CCCCCCC)OC(=O)CCCN(C)C] |
| | 4-dodecylhexadecyl 6-((4-(dimethylamino)butanoyl)oxy)tridecanoate |
| 83 | 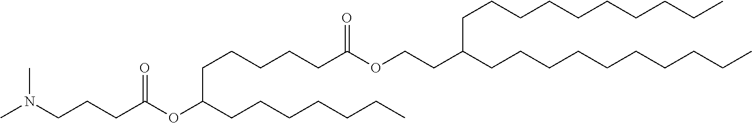 [SMILES: CCCCCCCCCCC(CCCCCCCCCC)CCOC(=O)CCCCCC(CCCCCCC)OC(=O)CCCN(C)C] |
| | 3-decyltridecyl 7-((4-(dimethylamino)butanoyl)oxy)tetradecanoate |
| 84 | 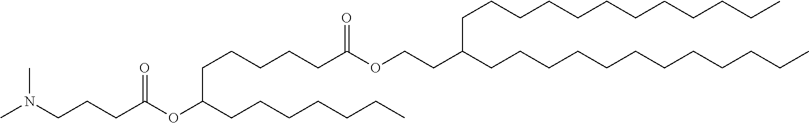 [SMILES: CCCCCCCCCCCCC(CCCCCCCCCCCC)CCOC(=O)CCCCCC(CCCCCCC)OC(=O)CCCN(C)C] |
| | 3-dodecylpentadecyl 7-((4-(dimethylamino)butanoyl)oxy)tetradecanoate |
| 85 | 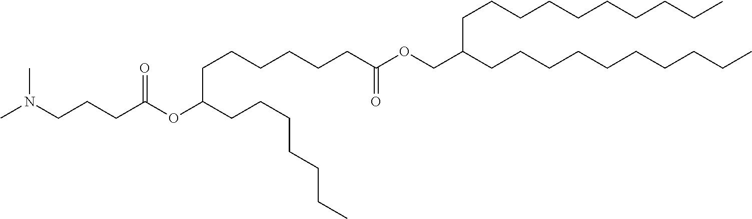 [SMILES: CCCCCCCCCCC(CCCCCCCCCC)COC(=O)CCCCCCC(CCCCCCC)OC(=O)CCCN(C)C] |
| | 2-decyldodecyl 8-((4-(dimethylamino)butanoyl)oxy)pentadecanoate |
| 86 | 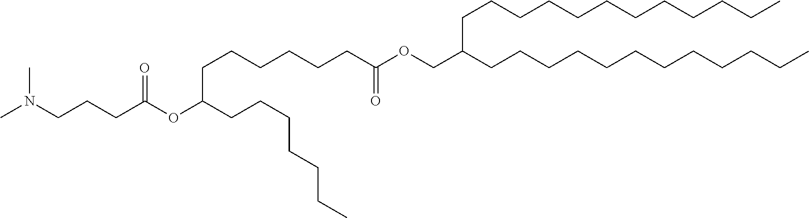 [SMILES: CCCCCCCCCCCCC(CCCCCCCCCCCC)COC(=O)CCCCCCC(CCCCCCC)OC(=O)CCCN(C)C] |
| | 2-dodecyltetradecyl 8-((4-(dimethylamino)butanoyl)oxy)pentadecanoate |
| 87 | 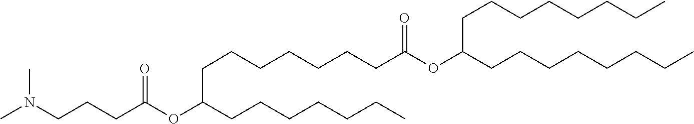 [SMILES: CCCCCCCCC(CCCCCCCC)OC(=O)CCCCCCCC(CCCCCCC)OC(=O)CCCN(C)C] |
| | heptadecan-9-yl 9-((4-(dimethylamino)butanoyl)oxy)hexadecanoate |
| 88 | 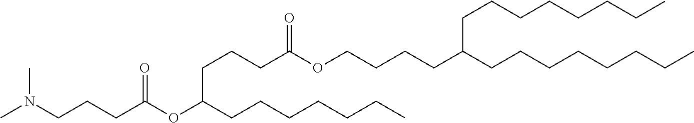 [SMILES: CCCCCCCCC(CCCCCCCC)CCCCOC(=O)CCCC(CCCCCCC)OC(=O)CCCN(C)C] |
| | 5-octyltridecyl 5-((4-(dimethylamino)butanoyl)oxy)dodecanoate |
| 89 | 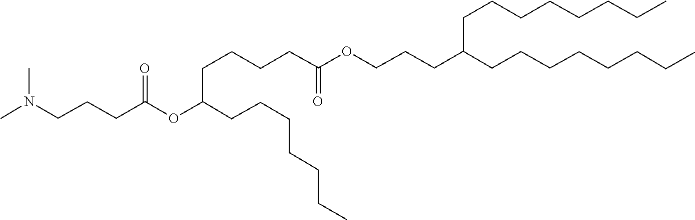 [SMILES: CCCCCCCCC(CCCCCCCC)CCCOC(=O)CCCCC(CCCCCCC)OC(=O)CCCN(C)C] |
| | 4-octyldodecyl 6-((4-(dimethylamino)butanoyl)oxy)tridecanoate |
| 90 | 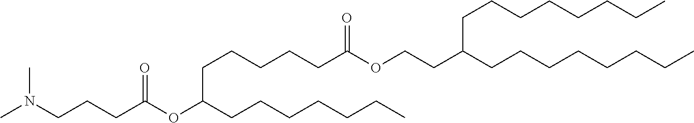 [SMILES: CCCCCCCCC(CCCCCCCC)CCOC(=O)CCCCCC(CCCCCCC)OC(=O)CCCN(C)C] |
| | 3-octylundecyl 7-((4-(dimethylamino)butanoyl)oxy)tetradecanoate |
| 91 | 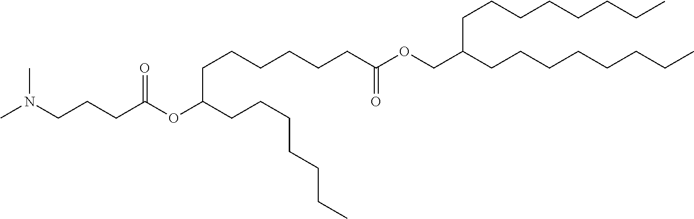 [SMILES: CCCCCCCCC(CCCCCCCC)COC(=O)CCCCCCC(CCCCCCC)OC(=O)CCCN(C)C] |
| | 2-octyldecyl 8-((4-(dimethylamino)butanoyl)oxy)pentadecanoate |
| 92 | 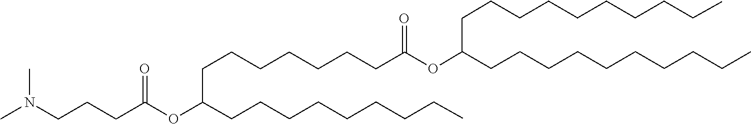 [SMILES: CCCCCCCCCCC(CCCCCCCCCC)OC(=O)CCCCCCCC(CCCCCCCCC)OC(=O)CCCN(C)C] |
| | henicosan-11-yl 9-((4-(dimethylamino)butanoyl)oxy)octadecanoate |
| 93 | 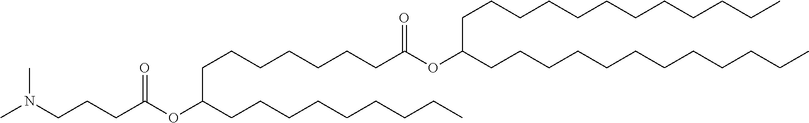 [SMILES: CCCCCCCCCCCCC(CCCCCCCCCCCC)OC(=O)CCCCCCCC(CCCCCCCCC)OC(=O)CCCN(C)C] |
| | pentacosan-13-yl 9-((4-(dimethylamino)butanoyl)oxy)octadecanoate |
| 94 | 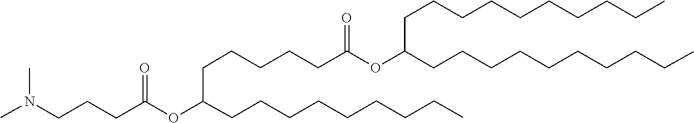 [SMILES: CCCCCCCCCCC(CCCCCCCCCC)OC(=O)CCCCCC(CCCCCCCCC)OC(=O)CCCN(C)C] |
| | henicosan-11-yl 7-((4-(dimethylamino)butanoyl)oxy)hexadecanoate |
| 95 | 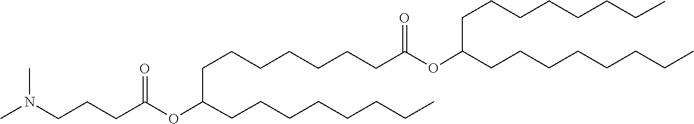 [SMILES: CCCCCCCCC(CCCCCCCC)OC(=O)CCCCCCCC(CCCCCCCC)OC(=O)CCCN(C)C] |
| | heptadecan-9-yl 9-((4-(dimethylamino)butanoyl)oxy)heptadecanoate |
| 96 | 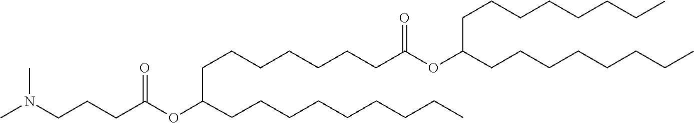 [SMILES: CCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)octadecanoate |
| 97 | 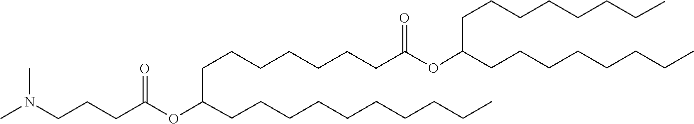 [SMILES: CCCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)nonadecanoate |
| 98 | 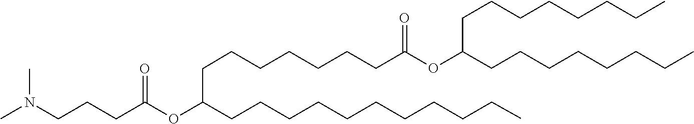 [SMILES: CCCCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)icosanoate |
| 99 | 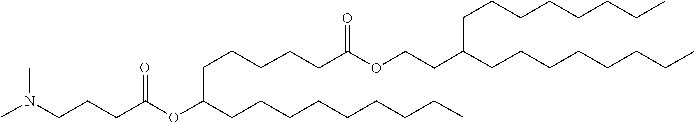 [SMILES: CCCCCCCCCC(CCCCCC(=O)OCCC(CCCCCCCC)CCCCCCCC)OC(=O)CCCN(C)C] |
| | 3-octylundecyl 7-((4-(dimethylamino)butanoyl)oxy)hexadecanoate |
| 100 | 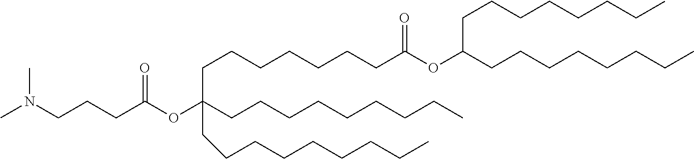 [SMILES: CCCCCCCCCC(CCCCCCCCC)(CCCCCCCC(=O)OC(CCCCCCCC)CCCCCCCC)OC(=O)CCCN(C)C] |
| | heptadecan-9-yl 9-((4-(dimethylamino)butanoyl)oxy)-9-nonyloctadecanoate |
| 101 | 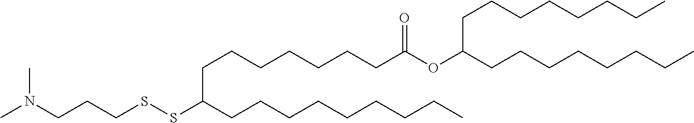 [SMILES: CCCCCCCCCC(CCCCCCCC(=O)OC(CCCCCCCC)CCCCCCCC)SSCCCN(C)C] |
| | heptadecan-9-yl 9-((3-(dimethylamino)propyl)disulfaneyl)octadecanoate |

### Formula (XX)

In some aspects, the cationic lipids are of the Formula (XX):

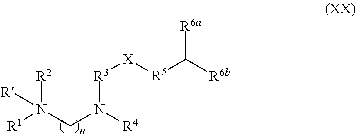

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;

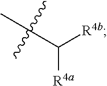

R⁴ is C₁-C₁₆ unbranched alkyl, C₂-C₁₆ unbranched alkenyl, or R^{4a}; wherein:
   R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{4b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment, in the cationic lipid according to the first embodiment, or a pharmaceutically acceptable salt thereof, X is —OC(═O)—, —SC(═O)—, —OC(═S)—, —C(═O)O—, —C(═O)S—, or —S—S—; and all other remaining variables are as described for Formula (XX) or the first embodiment.

In a third embodiment, the cationic lipid of the present disclosure is represented by Formula (XXI):

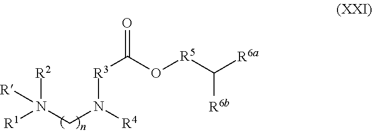

or a pharmaceutically acceptable salt thereof, wherein n is an integer selected from 1, 2, 3, and 4; and all other remaining variables are as described for Formula (XX) or any one of the preceding embodiments. In an alternative third embodiment, n is an integer selected from 1, 2, and 3; and all other remaining variables are as described for Formula (XX) or any one of the preceding embodiments.

In a fourth embodiment, the cationic lipid of the present disclosure is represented by Formula (XXII):

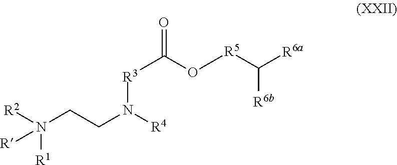

or a pharmaceutically acceptable salt thereof, and all other remaining variables are as described for Formula (XX), Formula (XXI) or any one of the preceding embodiments.

In a fifth embodiment, in the cationic lipid according to the first embodiment, or a pharmaceutically acceptable salt thereof, R¹ and R²are each independently hydrogen or C₁-C₂ alkyl, or C₂-C₃ alkenyl; or R′, R¹, and R² are each independently hydrogen, C₁-C₂ alkyl; and all other remaining variables are as described for Formula (XX), Formula (XXI) or any one of the preceding embodiments.

In a sixth embodiment, the cationic lipid of the present disclosure is represented by Formula (XXII):

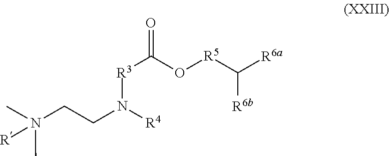

or a pharmaceutically acceptable salt thereof, and all other remaining variables are as described for Formula (XX), Formula (XXI), Formula (XXII) or any one of the preceding embodiments.

In a seventh embodiment, in the cationic lipid according to Formula (XX), Formula (XXI), Formula (XXII), Formula (XXIII) or any one of the preceding embodiments, or a pharmaceutically acceptable salt thereof, R is absent or C₁-C₅ alkylene; or R⁵ is absent, C₁-C₆ alkylene, or C₂-C₆ alkenylene; or R is absent, C₁-C₄ alkylene, or C₂-C₄ alkenylene; or R⁵ is absent; or R⁵ is C₆ alkylene, C₈ alkylene, C₄ alkylene, C₃ alkylene, C₂ alkylene, C₁ alkylene, C₆ alkenylene, C₈ alkenylene, C₄ alkenylene, C₃ alkenylene, or C₂ alkenylene; and all other remaining variables are as described for Formula (XX), Formula (XXI), Formula (XXII), Formula (XXIII) or any one of the preceding embodiments.

In an eighth embodiment, the cationic lipid of the present disclosure is represented by Formula (XXIV):

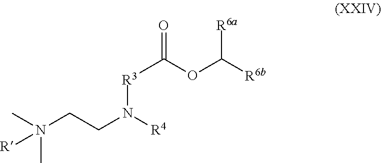

or a pharmaceutically acceptable salt thereof; and all other remaining variables are as described for Formula (XX), Formula (XXI), Formula (XXII), Formula (XXIII) or any one of the preceding embodiments.

In a ninth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R⁴ is C₁-C₁₄ unbranched alkyl, C₂-C₁₄ unbranched alkenyl, or

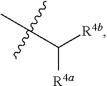

wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₁₂ unbranched alkyl or C₈-C₁₂ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₅ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₅ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

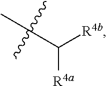

wherein R^{4a} and R^{4b} are each independently C₂-C₁₀ unbranched alkyl or C₂-C₁o unbranched alkenyl; or R⁴ is

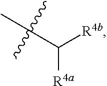

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₅ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁ unbranched alkenyl, C₁₀ unbranched alkenyl, C₉ unbranched alkenyl, C₅ unbranched alkenyl, C₇ unbranched alkenyl, C₆ unbranched alkenyl, C₅ unbranched alkenyl, C₄ unbranched alkenyl, C₃ unbranched alkenyl, or C₂alkenyl; and all other remaining variables are as described for Formula (XX), Formula (XXI), Formula (XXII), Formula (XXIII), Formula (XXIV) or any one of the preceding embodiments.

In a tenth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R³ is C₃-C₅ alkylene or C₃-C₅ alkenylene, C₃-C₇ alkylene or C₃-C₇ alkenylene, or C₃-C₅ alkylene or C₃-C₅ alkenylene,; or R³ is C₅ alkylene, or C₇ alkylene, or C₆ alkylene, or C₅ alkylene, or C₄ alkylene, or C₃ alkylene, or C₁ alkylene, or C₅ alkenylene, or C₇ alkenylene, or C₆ alkenylene, or C₅ alkenylene, or C₄ alkenylene, or C₃ alkenylene; and all other remaining variables are as described for Formula (XX), Formula (XXI), Formula (XXII), Formula (XXIII), Formula (XXIV) or any one of the preceding embodiments.

In an eleventh embodiment, in the cationic lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} are each independently C₇-C₁₂ alkyl or C₇-C₁₂ alkenyl; or R^{6a} and R^{4b} are each independently C₅-C₁₀ alkyl or C₅-C₁₀ alkenyl; or R^{6a} and R^{6b} are each independently C₁₂ alkyl, C₁₁ alkyl, C₁₀ alkyl, C₉ alkyl, C₅ alkyl, C₇ alkyl, C₁₂ alkenyl, C₁₁ alkenyl, C₁₀ alkenyl, C₉ alkenyl, C₅ alkenyl, or C₇ alkenyl; and all other remaining variables are as described for Formula (XX), Formula (XXI), Formula (XXII), Formula (XXIII), Formula (XXIV) or any one of the preceding embodiments.

In a twelfth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} contain an equal number of carbon atoms with each other; or R^{6a} and R^{6b} are the same; or R^{6a} and R^{6b} are both C₁₂ alkyl, C₁₁ alkyl, C₁₀ alkyl, C₉ alkyl, C₈ alkyl, C₇ alkyl, C₁₂ alkenyl, C₁₁ alkenyl, C₁₀ alkenyl, C₉ alkenyl, C₅ alkenyl, or C₇ alkenyl; and all other remaining variables are as described for Formula (XX), Formula (XXI), Formula (XXII), Formula (XXIII), Formula (XXIV) or any one of the preceding embodiments.

In a thirteenth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and Rib differs by one carbon atom; or R^{6a} is C₇ alkyl and R^{6a} is C₈ alkyl, R^{6a} is C₅ alkyl and R^{6a} is C₇ alkyl, R^{6a} is C₈ alkyl and R^{6a} is C₉ alkyl, R^{6a} is C₉ alkyl and R^{6a} is C₅ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₅ alkyl and R^{6a} is C₁₀ alkyl, R^{6a} is C₁₀ alkyl and R^{6a} is C₅ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, etc.; and all other remaining variables are as described for Formula (XX), Formula (XXI), Formula (XXII), Formula (XXIII), Formula (XXIV) or any one of the preceding embodiments.

In a fourteenth embodiment, in the cationic lipid according to Formula (XX), Formula (XXI), Formula (XXII), Formula (XXIII), Formula (XXIV) or any one of the preceding embodiments, or a pharmaceutically acceptable salt thereof, R′ is absent; and all other remaining variables are as described for Formula (XX), Formula (XXI), Formula (XXII), Formula (XXIII), Formula (XXIV) or any one of the preceding embodiments.

In one embodiment, the cationic lipid of the present disclosure or the cationic lipid of Formula (XX), Formula (XXI), Formula (XXII), Formula (XXIII), Formula (XXIV) is any one lipid selected from the lipids in Table 8 or a pharmaceutically acceptable salt thereof:

TABLE 8
Exemplary lipids of Formula (XX), Formula (XXI), Formula (XXII), Formula (XXIII), Formula (XXIV)

| Lipid | |
|---|---|
| No | Lipid Structure and Name |
| 102 | 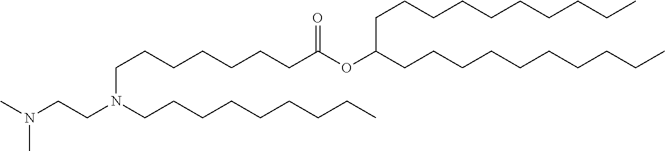 [SMILES: CCCCCCCCCCC(CCCCCCCCCC)OC(=O)CCCCCCCN(CCCCCCCCC)CCN(C)C] |
| | henicosan-11-yl 8-((2-(dimethylamino)ethyl)(nonyl)amino)octanoate |
| 103 | 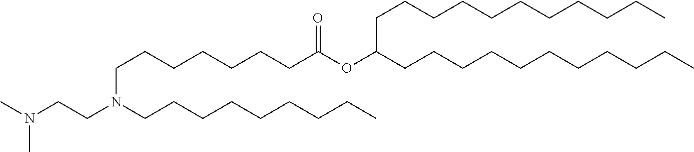 [SMILES: CCCCCCCCCCCC(CCCCCCCCCCC)OC(=O)CCCCCCCN(CCCCCCCCC)CCN(C)C] |
| | tricosan-12-yl 8-((2-(dimethylamino)ethyl)(nonyl)amino)octanoate |
| 104 | 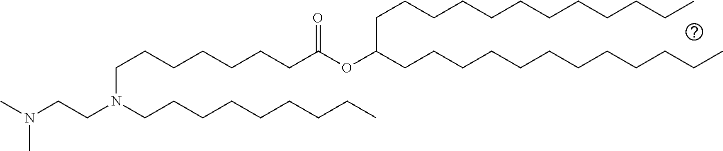 [SMILES: CCCCCCCCCCCCC(CCCCCCCCCCCC)OC(=O)CCCCCCCN(CCCCCCCCC)CCN(C)C] |
| | pentacosan-13-y1 8-((2-(dimethylamino)ethyl)(nonyl)amino)octanoate |
| 105 | 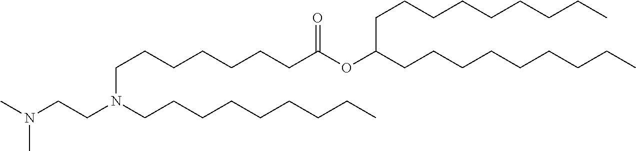 [SMILES: CCCCCCCCCC(CCCCCCCCC)OC(=O)CCCCCCCN(CCCCCCCCC)CCN(C)C] |
| | nonadecan-10-yl 8-((2-(dimethylamino)ethyl)(nonyl)amino)octanoate |
| 106 | 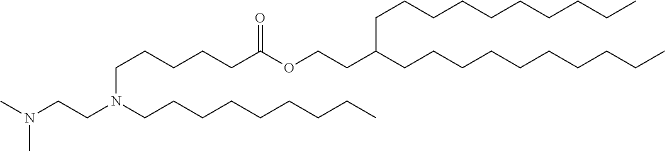 [SMILES: CCCCCCCCCCC(CCCCCCCCCC)CCOC(=O)CCCCCN(CCCCCCCCC)CCN(C)C] |
| | 3-decyltridecyl 6-((2-(dimethylamino)ethyl)(nonyl)amino)hexanoate |
| 107 | 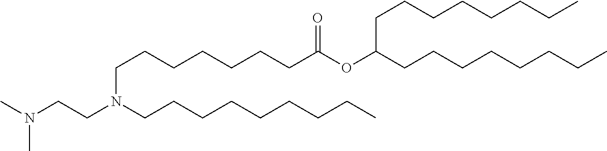 [SMILES: CCCCCCCCCN(CCCCCCCC(=O)OC(CCCCCCCC)CCCCCCCC)CCN(C)C] |
| | heptadecan-9-yl 8-((2-(dimethylamino)ethyl)(nonyl)amino)octanoate |
| 108 | 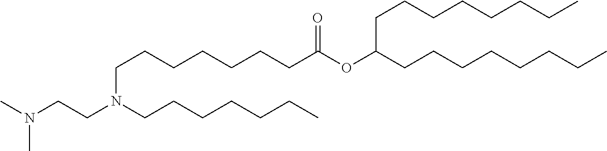 [SMILES: CCCCCCCCC(CCCCCCCC)OC(=O)CCCCCCCN(CCCCCCC)CCN(C)C] |
| | heptadecan-9-yl 8-((2-(dimethylamino)ethyl)(heptyl)amino)octanoate |
| 109 | 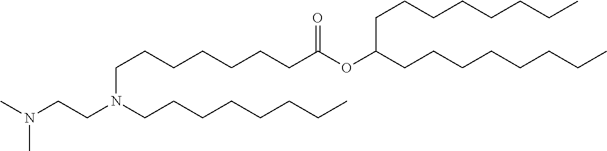 [SMILES: CCCCCCCCC(CCCCCCCC)OC(=O)CCCCCCCN(CCCCCCCC)CCN(C)C] |
| | heptadecan-9-yl 8-((2-(dimethylamino)ethyl)(octyl)amino)octanoate |
| 110 | 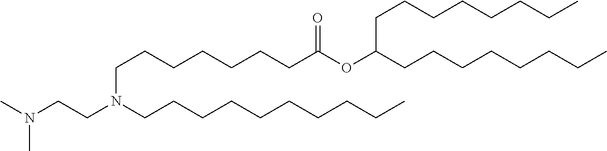 [SMILES: CCCCCCCCCCN(CCCCCCCC(=O)OC(CCCCCCCC)CCCCCCCC)CCN(C)C] |
| | heptadecan-9-y1 8-(decyl(2-(dimethylamino)ethyl)amino)octanoate |
| 111 | 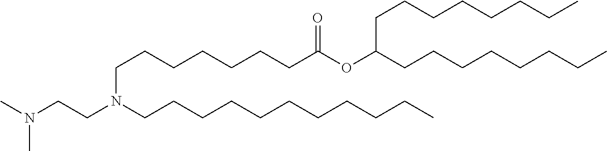 [SMILES: CCCCCCCCCCCN(CCCCCCCC(=O)OC(CCCCCCCC)CCCCCCCC)CCN(C)C] |
| | heptadecan-9-yl 8-((2-(dimethylamino)ethyl)(undecyl)amino)octanoate |
| 112 | 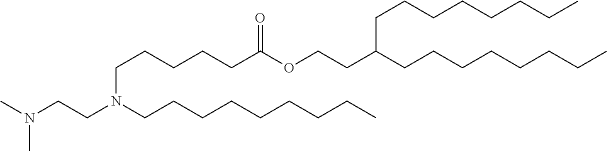 [SMILES: CCCCCCCCCN(CCCCCC(=O)OCCC(CCCCCCCC)CCCCCCCC)CCN(C)C] |
| | 3-octylundecyl 6-((2-(dimethylamino)ethyl)(nonyl)amino)hexanoate |

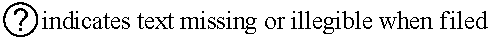

Specific examples are provided in the exemplification section below and are included as part of the ionizable lipids described herein. Pharmaceutically acceptable salts as well as neutral forms are also included.

### Cleavable Lipids

According to some embodiments, provided herein are pharmaceutical compositions comprising a lipid nanoparticle (LNP), a therapeutic nucleic acid (TNA) and at least one pharmaceutically acceptable excipient, wherein the LNP comprises at least one lipid, and wherein the LNP is capable of delivering the TNA to a retinal cell. As used herein, the term “cleavable lipid” refers to a cationic lipid comprising a disulfide bond (“SS”) cleavable unit. In one embodiment, SS-cleavable lipids comprise atertiary amine, which responds to an acidic compartment (e.g., an endosome or lysosome) for membrane destabilization and a disulfide bond that can cleave in a reductive environment (e.g., the cytoplasm). SS-cleavable lipids may include SS-cleavable and pH-activated lipid-like materials, such as ss-OP lipids, ssPalm lipids, ss-M lipids, ss-E lipids, ss-EC lipids, ss-LC lipids and ss-OC lipids, etc.

According to some embodiments, SS-cleavable lipids are described in International Patent Application Publication No. WO2019188867, incorporated by reference in its entirety herein.

According to some embodiments, the LNPs described herein range in size from about 20 to about 70 nm in mean diameter, for example, a mean diameter of from about 20 nm to about 70 nm, about 25 nm to about 70 nm, from about 30 nm to about 70 nm, from about 35 nm to about 70 nm, from about 40 nm to about 70 nm, from about 45 nm to about 80 nm, from about 50 nm to about 70 nm, from about 60 nm to about 70 nm, from about 65 nm to about 70 nm, or about 20 nm, about 25 nm, about 30 nm, about 35 nm, about 40 nm, about 45 nm, about 50 nm, about 55 nm, about 60 nm, about 65 nm, about 70 nm. According to some embodiments, the mean diameter of the LNPs is about 50 nm to about 70 nm. which is significantly smaller and therefore advantageous in targeting and circumventing immune responses. Moreover, the LNPs described herein can encapsulate greater than about 60% to about 90% of double stranded DNA, like ceDNA. According to some embodiments, the LNPs described herein can encapsulate greater than about 60% of double stranded DNA, like ceDNA, greater than about 65% of double stranded DNA, like ceDNA, greater than about 70% of double stranded DNA, like ceDNA, greater than about 75% of double stranded DNA, like ceDNA, greater than about 80% of double stranded DNA, like ceDNA, greater than about 85% of double stranded DNA, like ceDNA, or greater than about 90% of double stranded DNA, like ceDNA.

The lipid particles (e.g., LNPs, wherein the LNP comprises at least one lipid) described herein can advantageously be used to increase delivery of nucleic acids (e.g., ceDNA, mRNA) to target cells/tissues compared to LNPs produced by other processes, and compared to other lipids, e.g., ionizable cationic lipids. Thus, the lipid particles (e.g., LNPs, wherein the LNP comprises at least one lipid) described herein provided maximum nucleic acid delivery compared to lipid particles prepared by processes and methods known in the art.

In one embodiment, a cleavable lipid may comprise three components: an amine head group, a linker group, and a hydrophobic tail(s). In one embodiment, the cleavable lipid comprises one or more phenyl ester bonds, one of more tertiary amino groups, and a disulfide bond. The tertiary amine groups provide pH responsiveness and induce endosomal escape, the phenyl ester bonds enhance the degradability of the structure (self-degradability) and the disulfide bond cleaves in a reductive environment.

In one embodiment, the cleavable lipid is an SS-cleavable lipid. In one embodiment, the SS-cleavable lipid comprises the structure shown below of Lipid A:

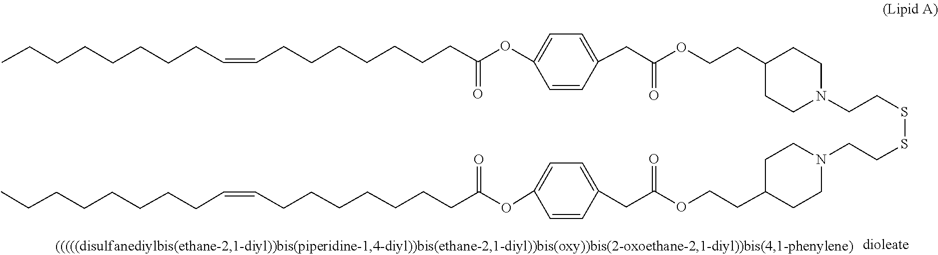

In one embodiment, the SS-cleavable lipid is an SS-cleavable and pH-activated lipid-like material (ssPalm). ssPalm lipids are well known in the art. For example, see Togashi et al., Journal of Controlled Release, 279 (2018) 262-270, the entire contents of which are incorporated herein by reference. In one embodiment, the ssPalm is an ssPalmM lipid comprising the structure of Lipid B.

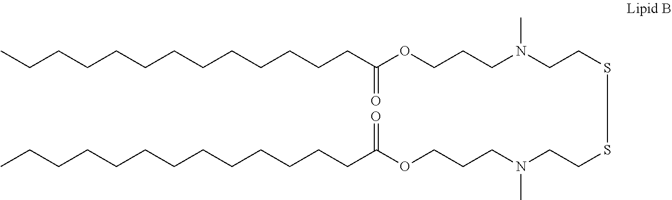 [SMILES: CCCCCCCCCCCCCC(=O)OCCCN(C)CCSCSCCN(C)CCCOC(=O)CCCCCCCCCCCCC]

In one embodiment, the ssPalmE lipid is a ssPalmE-P4-C2 lipid, comprising the structure of Lipid C.

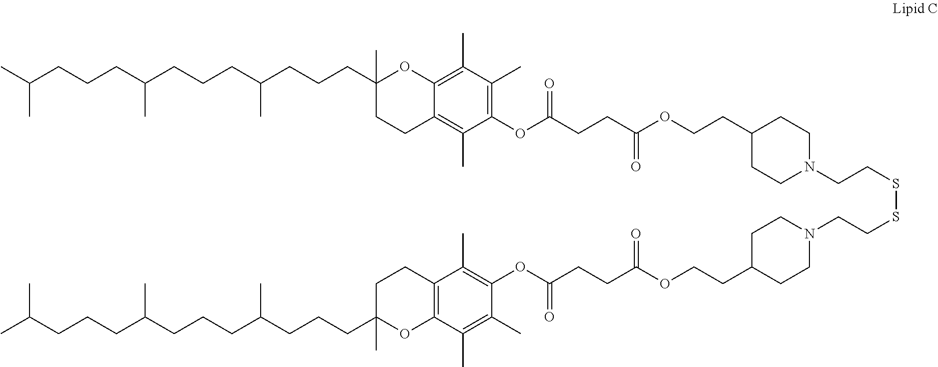 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the ssPalmE lipid is a ssPalmE-Paz4-C2 lipid, comprising the structure of Lipid D.

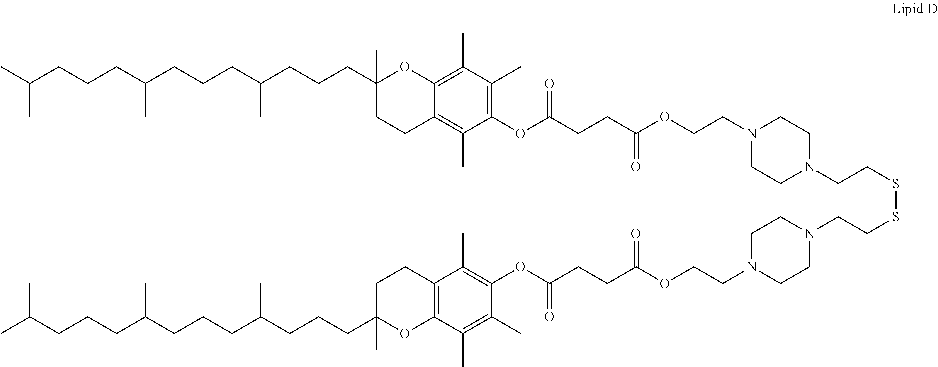 [SMILES: Cc1c(C)c2c(c(C)c1OC(=O)CCC(=O)OCCN1CCN(CCSSCCN3CCN(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-M lipid. In one embodiment, an ss-M lipid comprises the structure shown in Lipid E below:

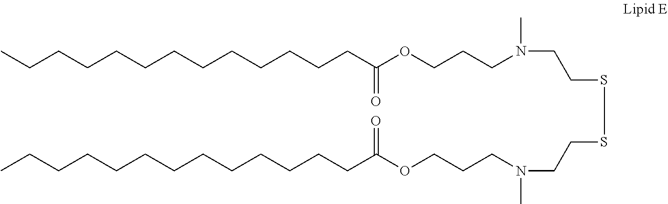 [SMILES: CCCCCCCCCCCCCC(=O)OCCCN(C)CCSSCCN(C)CCCOC(=O)CCCCCCCCCCCCC]

In one embodiment, the cleavable lipid is an ss-E lipid. In one embodiment, an ss-E lipid comprises the structure shown in Lipid F below:

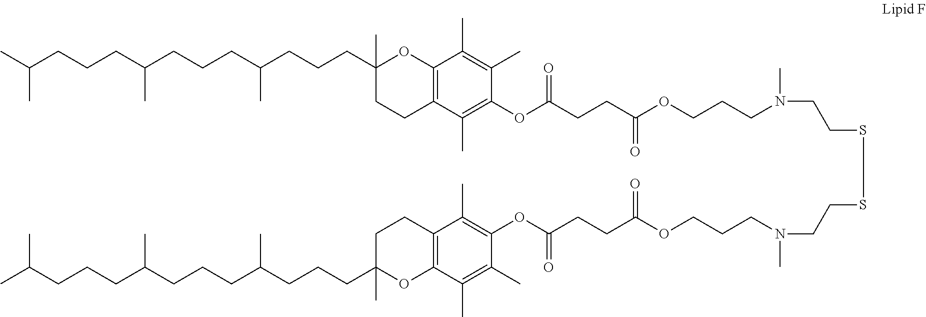 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In one embodiment, the cleavable lipid is an ss-EC lipid. In one embodiment, an ss-EC lipid comprises the structure shown in Lipid G below:

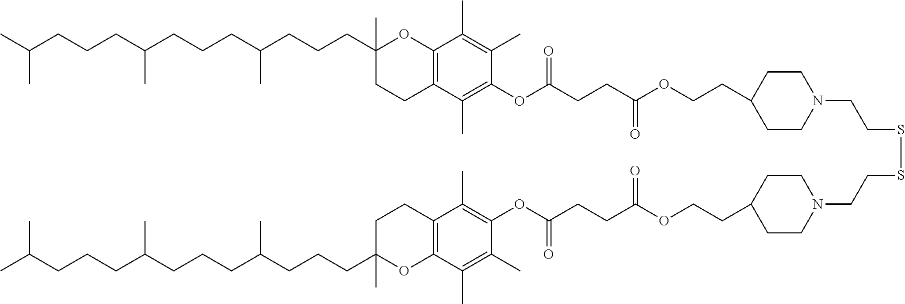 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-LC lipid. In one embodiment, an ss-LC lipid comprises the structure shown in Lipid H below:

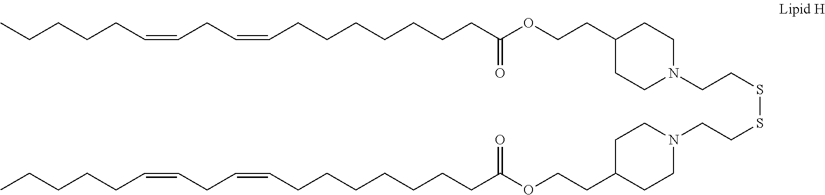 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In one embodiment, the cleavable lipid is an ss-OC lipid. In one embodiment, an ss-OC lipid comprises the structure shown in Lipid J below:

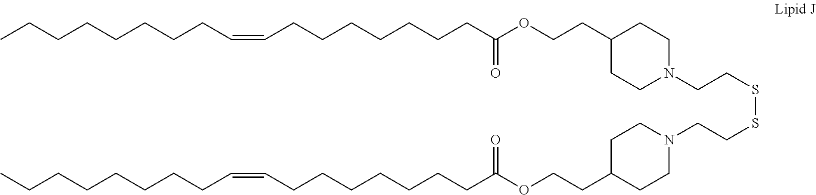 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

In some embodiments, a lipid nanoparticle of the present disclosure comprises Lipid A as listed above.

In some embodiments, a lipid nanoparticle of the present disclosure may comprise Lipid A, DOPC, cholesterol and PEG-DMG. In some embodiments, a lipid nanoparticle of the present disclosure may comprise Lipid A, DOPC, cholesterol and PEG2000-DMG.

In some embodiments, a lipid nanoparticle of the present disclosure may comprise Lipid A, DOPC, cholesterol, PEG₂₀₀₀-DMG and GalNAc. In further embodiments, the lipid nanoparticle may comprise Lipid A, DOPC, cholesterol, PEG₂₀₀₀-DMG and GalNAc with molar ratios of 50%:10%:38%:1.5%:0.5%, respectively.

In one embodiment, a lipid particle formulation is made and loaded with ceDNA obtained by the process as disclosed in International Patent Application No. PCT/US2018/050042, filed on Sep. 7, 2018, which is incorporated by reference in its entirety herein. This can be accomplished by high energy mixing of ethanolic lipids with aqueous ceDNA at low pH which protonates the lipid and provides favorable energetics for ceDNA/lipid association and nucleation of particles. The particles can be further stabilized through aqueous dilution and removal of the organic solvent. The particles can be concentrated to the desired level. In one embodiment, the disclosure provides a ceDNA lipid particle comprising a lipid of Formula I prepared by a process as described in Example 2 of U.S. Provisional Application No. 63/194,620, incorporated by reference in its entirety herein.

Generally, the lipid particles are prepared at a total lipid to ceDNA (mass or weight) ratio of from about 10:1 to 60:1. In some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles are prepared at a ceDNA (mass or weight) to total lipid ratio of about 60:1. According to some embodiments, the lipid particles are prepared at a ceDNA (mass or weight) to total lipid ratio of about 30:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

In some embodiments, the lipid nanoparticle comprises an agent for condensing and/or encapsulating nucleic acid cargo, such as ceDNA. Such an agent is also referred to as a condensing or encapsulating agent herein. Without limitations, any compound known in the art for condensing and/or encapsulating nucleic acids can be used as long as it is non-fusogenic. In other words, an agent capable of condensing and/or encapsulating the nucleic acid cargo, such as ceDNA, but having little or no fusogenic activity. Without wishing to be bound by theory, a condensing agent may have some fusogenic activity when not condensing/encapsulating a nucleic acid, such as ceDNA, but a nucleic acid encapsulating lipid nanoparticle formed with said condensing agent can be non-fusogenic.

According to some embodiments, the LNPs described herein can encapsulate greater than about 60% of rigid double stranded DNA, like ceDNA, greater than about 65% of rigid double stranded DNA, like ceDNA, greater than about 70% of rigid double stranded DNA, like ceDNA, greater than about 75% of rigid double stranded DNA, like ceDNA, greater than about 80% of rigid double stranded DNA, like ceDNA, greater than about 85% of rigid double stranded DNA, like ceDNA, or greater than about 90% of rigid double stranded DNA, like ceDNA.

The cationic lipid is typically employed to condense the nucleic acid cargo, e.g., ceDNA at low pH and to drive membrane association and fusogenicity. Generally, cationic lipids are lipids comprising at least one amino group that is positively charged or becomes protonated under acidic conditions, for example at pH of 6.5 or lower. Cationic lipids may also be ionizable lipids, e.g., ionizable cationic lipids. By a “non-fusogenic cationic lipid” is meant a cationic lipid that can condense and/or encapsulate the nucleic acid cargo, such as ceDNA, but does not have, or has very little, fusogenic activity.

In one embodiment, the cationic lipid can comprise 20-90% (mol) of the total lipid present in the lipid particles (e.g., lipid nanoparticles). For example, cationic lipid molar content can be 20-70% (mol), 30-60% (mol), 40-60% (mol), 40-55% (mol) or 45-55% (mol) of the total lipid present in the lipid particle (e.g., lipid nanoparticles). In some embodiments, cationic lipid comprises from about 50 mol % to about 90 mol % of the total lipid present in the lipid particles described herein.

In one embodiment, the SS-cleavable lipid is not MC3 (6Z,9Z,28Z,3 lZ)-heptatriaconta-6,9,28,3 1-tetraen-19-yl-4-(dimethylamino)butanoate (DLin-MC3-DMA or MC3). DLin-MC3-DMA is described in Jayaraman et al., Angew. Chem. Int. Ed Engl. (2012), 51(34): 8529-8533, the contents of which is incorporated herein by reference in its entirety. The structure of D-Lin-MC3-DMA (MC3) is shown below as Lipid K:

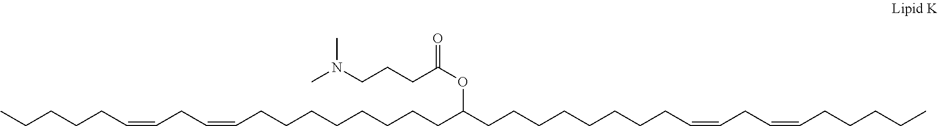 [SMILES: CCCCC/C=C\C/C=C\CCCCCCCCC(CCCCCCCC/C=C\C/C=C\CCCCC)OC(=O)CCCN(C)C]

In one embodiment, the cleavable lipid is not the lipid ATX-002. The lipid ATX-002 is described in WO2015/074085, the content of which is incorporated herein by reference in its entirety. In one embodiment, the cleavable lipid is not (13Z.16Z)-/V,/V-dimethyl-3-nonyldocosa-13,16-dien-1-amine (Compound 32). Compound 32 is described in WO2012/040184, the contents of which is incorporated herein by reference in its entirety. In one embodiment, the cleavable lipid is not Compound 6 or Compound 22. Compounds 6 and 22 are described in WO2015/199952, the content of which is incorporated herein by reference in its entirety.

Non-limiting examples of cationic lipids include SS-cleavable and pH-activated lipid-like material-OP (ss-OP; Formula I), SS-cleavable and pH-activated lipid-like material-M (SS-M; Formula V), SS-cleavable and pH-activated lipid-like material-E (SS-E; Formula VI), SS-cleavable and pH-activated lipid-like material-EC (SS-EC; Formula VII), SS-cleavable and pH-activated lipid-like material-LC (SS-LC; Formula VIII), SS-cleavable and pH-activated lipid-like material-OC (SS-OC; Formula IX), polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3b-[N-(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., ceDNA or CELiD) can also be complexed with, e.g., poly (L-lysine) or avidin and lipids can, or cannot, be included in this mixture, e.g., steryl-poly (L-lysine).

In one embodiment, the cationic lipid is ss-OP of Formula I. In another embodiment, the cationic lipid SS-PAZ of Formula II.

In one embodiment, a ceDNA vector as disclosed herein is delivered using a cationic lipid described in U.S. Pat. No. 8,158,601, or a polyamine compound or lipid as described in U.S. Pat. No. 8,034,376.

### B. Non-cationic Lipids

In one embodiment, the lipid particles (e.g., LNPs, wherein the LNP comprises at least one lipid) can further comprise a non-cationic lipid. The non-cationic lipid can serve to increase fusogenicity and also increase stability of the LNP during formation. Non-cationic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid particles described herein include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In one embodiment, the non-cationic lipid is a phospholipid. In one embodiment, the non-cationic lipid is selected from the group consisting of DSPC, DPPC, DMPC, DOPC, POPC, DOPE, and SM. In some embodiments, the non-cationic lipid is DSPC. In other embodiments, the non-cationic lipid is DOPC. In other embodiments, the non-cationic lipid is DOPE.

In some embodiments, the non-cationic lipid can comprise 0-20% (mol) of the total lipid present in the lipid nanoparticle. In some embodiments, the non-cationic lipid content is 0.5-15% (mol) of the total lipid present in the lipid particle (e.g., lipid nanoparticle). In some embodiments, the non-cationic lipid content is 5-12% (mol) of the total lipid present in the lipid particle (e.g., lipid nanoparticle). In some embodiments, the non-cationic lipid content is 5-10% (mol) of the total lipid present in the lipid particle (e.g., lipid nanoparticle). In one embodiment, the non-cationic lipid content is about 6% (mol) of the total lipid present in the lipid particle (e.g., lipid nanoparticle). In one embodiment, the non-cationic lipid content is about 7.0% (mol) of the total lipid present in the lipid particle (e.g., lipid nanoparticle). In one embodiment, the non-cationic lipid content is about 7.5% (mol) of the total lipid present in the lipid particle (e.g., lipid nanoparticle). In one embodiment, the non-cationic lipid content is about 8.0% (mol) of the total lipid present in the lipid particle (e.g., lipid nanoparticle). In one embodiment, the non-cationic lipid content is about 9.0% (mol) of the total lipid present in the lipid particle (e.g., lipid nanoparticle). In some embodiments, the non-cationic lipid content is about 10% (mol) of the total lipid present in the lipid particle (e.g., lipid nanoparticle). In one embodiment, the non-cationic lipid content is about 11% (mol) of the total lipid present in the lipid particle (e.g., lipid nanoparticle).

Exemplary non-cationic lipids are described in International Patent Application Publication No. WO2017/099823 and US Patent Application Publication No. US2018/0028664, the contents of both of which are incorporated herein by reference in their entirety.

In one embodiment, the lipid particles (e.g., lipid nanoparticles) can further comprise a component, such as a sterol, to provide membrane integrity and stability of the lipid particle. In one embodiment, an exemplary sterol that can be used in the lipid particle is cholesterol, or a derivative thereof. Non-limiting examples of cholesterol derivatives include polar analogues such as 5a-cholestanol, 5p-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

Exemplary cholesterol derivatives are described in International Patent Application Publication No. WO2009/127060 and U.S. Patent Application Publication No. US2010/0130588, contents of both of which are incorporated herein by reference in their entirety.

In one embodiment, the component providing membrane integrity, such as a sterol, can comprise 0-50% (mol) of the total lipid present in the lipid particle (e.g., lipid nanoparticle). In some embodiments, such a component is 20-50% (mol) of the total lipid content of the lipid particle (e.g., lipid nanoparticle). In some embodiments, such a component is 30-40% (mol) of the total lipid content of the lipid particle (e.g., lipid nanoparticle). In some embodiments, such a component is 35-45% (mol) of the total lipid content of the lipid particle (e.g., lipid nanoparticle). In some embodiments, such a component is 38-42% (mol) of the total lipid content of the lipid particle (e.g., lipid nanoparticle).

In one embodiment, the lipid particle (e.g., lipid nanoparticle) can further comprise a polyethylene glycol (PEG) or a conjugated lipid molecule. Generally, these are used to inhibit aggregation of lipid particle (e.g., lipid nanoparticle) and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEGylated lipid, for example, a (methoxy polyethylene glycol)-conjugated lipid. In some other embodiments, the PEGylated lipid is PEG₂₀₀₀-DMG (dimyristoylglycerol).

Exemplary PEGylated lipids include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4—O-(2′,3′-di(tetradecanoyloxy)propyl-l-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In one embodiment, the PEG-DAA PEGylated lipid can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In one embodiment, the PEG-lipid can be selected from the group consisting of PEG-DMG, 1,2-dimyristoyl-sn-glycero-3-phosphoethanolamine-N-[methoxy(polyethylene glycol)-2000],

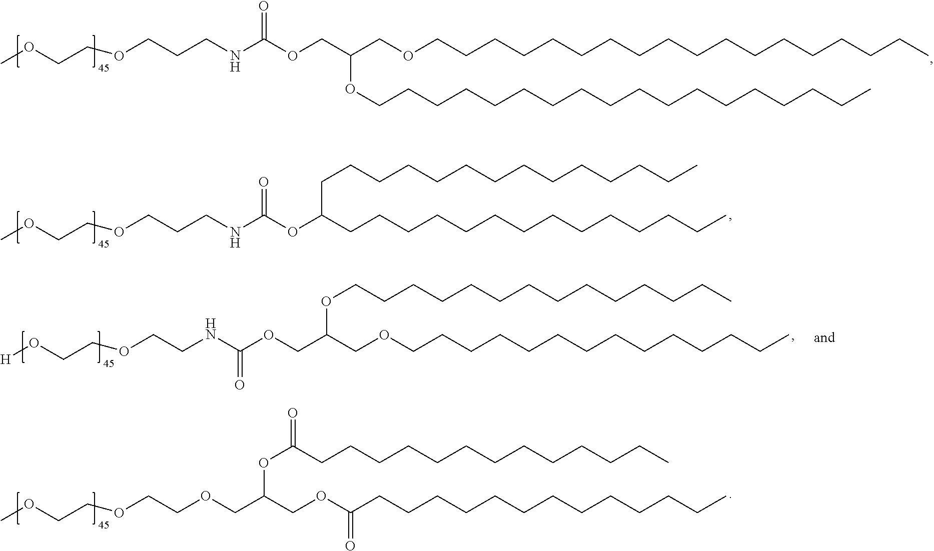

In some embodiments, the PEGylated lipid is selected from the group consisting N-(Carbonyl-methoxypolyethyleneglycoln)-1,2-dimyristoyl-sn-glycero-3-phosphoethanolamine (DMPE-PEGₙ, where n (representing PEG average molecular weight) is 350, 500, 750, 1000 or 2000), N-(Carbonyl-methoxypolyethyleneglycoln)-1,2-distearoyl-sn-glycero-3-phosphoethanolamine (DSPE-PEGₙ, where n is 350, 500, 750, 1000, 2000, or 5000), DSPE-polyglycelin-cyclohexyl-carboxylic acid, DSPE-polyglycelin-2-methylglutar-carboxylic acid, 1,2-Distearoyl-sn-Glycero-3-Phosphoethanolamine (DSPE) conjugated Polyethylene Glycol (DSPE-PEG-OH), polyethylene glycol-dimyristolglycerol (PEG-DMG), polyethylene glycol-distearoyl glycerol (PEG-DSG), or N-octanoyl-sphingosine-1-{succinyl[methoxy(polyethylene glycol)200011 (C₈ PEG2000 Ceramide). In some examples of DMPE-PEG,, where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some examples of DSPE-PEG,. where n (representing PEG average molecular weight) is 350, 500, 750, 1000, 2000, or 5000 the PEG-lipid is N-(Carbonyl-methoxypolyethyleneglycol 2000)-1,2-distearoyl-sn-glycero-3-phosphoethanolamine (DSPE-PEG 2,000). In some embodiments, the PEGylated lipid is DSPE-PEG-OH. In some embodiments, the PEGylated lipid is PEG-DMG. In some embodiments, the PEGylated lipid is PEG-DSG.

In one embodiment, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International Patent Application Publication Nos. WO 1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, U.S. Patent Application Publication Nos. US2003/0077829, US2005/0175682, US2008/0020058, US2011/0117125, US2013/0303587, US2018/0028664, US2015/03761 15, US2016/0376224, US2016/0317458, US2013/0303587, US2013/0303587, and US20110123453, and U.S. Pat. Nos. U.S. Pat. Nos. 5,885,613, 6,287,591, 6,320,017, and 6,586,559, the contents of all of which are incorporated herein by reference in their entireties.

In some embodiments, the PEGylated lipid can comprise 0-20% (mol) of the total lipid present in the lipid nanoparticle. In some embodiments, PEGylated lipid content is about 0.5-10% (mol). In some embodiments, PEGylated lipid content is about 1-5% (mol). In some embodiments, PEGylated lipid content is 2-4% (mol). In some embodiments, PEGylated lipid content is about 2-3% (mol). In some embodiments, PEGylated lipid content is about 1-3% (mol). In some embodiments, PEGylated lipid content is about 0.75-2.5% (mol). In some embodiments, PEGylated lipid content is about 0.75-2.0% (mol). In some embodiments, PEGylated lipid content is about 0.75-1.8% (mol). In some embodiments, PEGylated lipid content is about 1-2% (mol). In some embodiments, PEGylated lipid content is about 0.75-1.5% (mol). In some embodiments, PEGylated lipid content is about 1-1.8% (mol). In some embodiments, PEGylated lipid content is about 1-1.5% (mol). In some embodiments, PEGylated lipid content is about 1-1.3% (mol). In some embodiments, PEGylated lipid content is about 1-1.2% (mol). In some embodiments, PEGylated lipid content is about 0.75-1.5% (mol). In some embodiments, PEGylated lipid content is about 0.75-1.25% (mol). In some embodiments, PEGylated lipid content is about 1.5-1.8% (mol). In some embodiments, PEGylated lipid content is about 1.2-1.5% (mol). In one embodiment, PEGylated lipid content is about 2% (mol). In one embodiment, PEGylated lipid content is about 2.5% (mol). In some embodiments, PEGylated lipid content is about 3% (mol). In one embodiment, PEGylated lipid content is about 3.5% (mol). In one embodiment, PEGylated lipid content is about 4% (mol).

It is understood that molar ratios of the cationic lipid, e.g., ionizable cationic lipid, with the non-cationic-lipid, sterol, and PEGylated lipid can be varied as needed. For example, the lipid particle (e.g., lipid nanoparticle) can comprise 30-70% cationic lipid by mole or by total weight of the composition, 0-60% cholesterol by mole or by total weight of the composition, 0-30% non-cationic lipid by mole or by total weight of the composition and 2-5% PEGylated lipid by mole or by total weight of the composition. In one embodiment, the composition comprises 40-60% cationic lipid by mole or by total weight of the composition, 30-50% cholesterol by mole or by total weight of the composition, 5-15% non-cationic lipid by mole or by total weight of the composition and 2-5% PEG or the conjugated lipid by mole or by total weight of the composition. In one embodiment, the composition is 40-60% cationic lipid by mole or by total weight of the composition, 30-40% cholesterol by mole or by total weight of the composition, and 5-10% non-cationic lipid, by mole or by total weight of the composition and 2-5% PEGylated lipid by mole or by total weight of the composition. The composition may contain 60-70% cationic lipid by mole or by total weight of the composition, 25-35% cholesterol by mole or by total weight of the composition, 5-10% non-cationic-lipid by mole or by total weight of the composition and 2-5% PEGylated lipid by mole or by total weight of the composition. The composition may also contain up to 45-55% cationic lipid by mole or by total weight of the composition, 35-45% cholesterol by mole or by total weight of the composition, 2 to 15% non-cationic lipid by mole or by total weight of the composition, and 2-5% PEGylated lipid by mole or by total weight of the composition. The formulation may also be a lipid nanoparticle formulation, for example comprising 8-30% cationic lipid by mole or by total weight of the composition, 5-15% non-cationic lipid by mole or by total weight of the composition, and 0-40% cholesterol by mole or by total weight of the composition; 4-25% cationic lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% cationic lipid by mole or by total weight of the composition, 2-30% non-cationic lipid by mole or by total weight of the composition, 1 to 15% cholesterol by mole or by total weight of the composition, 2 to 35% PEGylated lipid by mole or by total weight of the composition, and 1-20% cholesterol by mole or by total weight of the composition; or even up to 90% cationic lipid by mole or by total weight of the composition and 2-10% non-cationic lipids by mole or by total weight of the composition, or even 100% cationic lipid by mole or by total weight of the composition. In some embodiments, the lipid particle formulation comprises cationic lipid, non-cationic phospholipid, cholesterol and a PEGylated lipid (conjugated lipid) in a molar ratio of about 50:9:38.5:2.5.

In one embodiment, the lipid particle (e.g., lipid nanoparticle) formulation comprises cationic lipid, non-cationic phospholipid, cholesterol and a PEGylated lipid (conjugated lipid) in a molar ratio of about 50:7:40:3.

In other aspects, the disclosure provides for a lipid nanoparticle formulation comprising phospholipids, lecithin, phosphatidylcholine and phosphatidylethanolamine.

In one embodiment, the lipid particle (e.g., lipid nanoparticle) comprises cationic lipid, non-cationic lipid (e.g., phospholipid), a sterol (e.g., cholesterol) and a PEGylated lipid (conjugated lipid), where the molar ratio of lipids ranges from 20 to 70 mole percent for the cationic lipid, with a target of 30-60, the mole percent of non-cationic lipid ranges from 0 to 30, with a target of 0 to 15, the mole percent of sterol ranges from 20 to 70, with a target of 30 to 50, and the mole percent of PEGylated lipid (conjugated lipid) ranges from 1 to 6, with a target of 2 to 5.

Lipid nanoparticles (LNPs) comprising ceDNA are disclosed in International Patent Application No. PCT/US2018/050042, filed on Sep. 7, 2018, which is incorporated herein in its entirety and envisioned for use in the methods and compositions as disclosed herein.

Lipid particle (e.g., lipid nanoparticle) size can be determined by quasi-elastic light scattering using a Malvern Zetasizer Nano ZS (Malvern, UK). According to some embodiments, LNP mean diameter as determined by light scattering is less than about 75 nm or less than about 70 nm. According to some embodiments, LNP mean diameter as determined by light scattering is between about 50 nm to about 75 nm or about 50 nm to about 70 nm.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al., Nature Biotechnology 28, 172-176 (20 1 0), both of which are incorporated by reference in their entireties). In one embodiment, the pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 mM stock solution in distilled water. Vesicles can be diluted to 24 mM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 mM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

In one embodiment, relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, a lipid particle (e.g., lipid nanoparticle) of the disclosure includes a lipid formulation that can be used to deliver a capsid-free, non-viral DNA vector to a target site of interest (e.g., cell, tissue, organ, and the like). Generally, the lipid particle (e.g., lipid nanoparticle) comprises capsid-free, non-viral DNA vector and a cationic lipid or a salt thereof.

In one embodiment, the lipid particle (e.g., lipid nanoparticle) comprises a cationic lipid/non-cationic-lipid/sterol/conjugated lipid at a molar ratio of 50:10:38.5:1.5. In one embodiment, the disclosure provides for a lipid particle (e.g., lipid nanoparticle) formulation comprising phospholipids, lecithin, phosphatidylcholine and phosphatidylethanolamine.

### III. Therapeutic Nucleic Acids

Aspects of the disclosure generally provide compositions (e.g., pharmaceutical compositions) comprising a lipid nanoparticle (LNP) and a therapeutic nucleic acid (TNA). According to some embodiments, the disclosure provides LNPs comprising a therapeutic nucleic acid (TNA) and at least one pharmaceutically acceptable excipient, wherein the LNP comprises at least one lipid, and wherein the LNP is capable of delivering the TNA to a retinal cell.

Illustrative therapeutic nucleic acids of the present disclosure can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), doggybone™, protelomere closed ended DNA, or dumbbell linear DNA), dicer-substrate dsRNA, small hairpin RNA (shRNA), asymmetrical interfering RNA (aiRNA), microRNA (miRNA), mRNA, tRNA, rRNA, gRNA, and DNA viral vectors, viral RNA vector, and any combination thereof.

siRNA or miRNA that can downregulate the intracellular levels of specific proteins through a process called RNA interference (RNAi) are also contemplated by the present disclosure to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson -capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the aspects and embodiments provided herein, the therapeutic nucleic acid can be a therapeutic RNA. Said therapeutic RNA can be an inhibitor of mRNA translation, agent of RNA interference (RNAi), catalytically active RNA molecule (ribozyme), transfer RNA (tRNA) or an RNA that binds an mRNA transcript (ASO), protein or other molecular ligand (aptamer). In any of the methods provided herein, the agent of RNAi can be a double-stranded RNA, single-stranded RNA, micro RNA, short interfering RNA, short hairpin RNA, or a triplex-forming oligonucleotide.

According to some embodiments, a therapeutic nucleic acid, when transcribed from a pharmaceutical composition comprising a LNP, as described herein, interferes with the ocular disorders of the invention by interfering with the translation or transcription of aberrant or excess proteins associated with the disorders of the invention.

According to some embodiments, the therapeutic nucleic acid is a mRNA. According to some embodiments, the mRNA is translated to a therapeutic polypeptide. The therapeutic polypeptide may provide, for example, a polypeptide and/or enzyme activity that is absent or present at reduced levels in a retinal cell or organism. For example, gene augmentation therapy for retinal degeneration caused by mutations in the RPE65 gene, showed success in clinical trials with an AAV delivery platform. Alternatively, the therapeutic polypeptide may provide a polypeptide and/or enzymatic activity that indirectly reverses the imbalance within the retinal cell.

According to some embodiments, the therapeutic nucleic acid encodes a therapeutic polypeptide. Non-limiting examples of nucleic acids encoding therapeutic polypeptides include: nucleic acids for the replacement of defective or mutated genes known to cause retinal disease, e.g., Prph2, RPE65, MERTK, RPGR, RP2, RPGRIP, CNGA3, CNGB3 and GNAT2. Other non-limiting examples of nucleic acids encoding therapeutic polypeptides include neurotrophic factors (e.g., GDNF, CNTF, FGF2, PEDF, EPO), anti-apoptotic genes (e.g. BCL2, BCL-X, NFκB), Anti-angiogenic factors (e.g., endostatin, angiostatin, sFlt), and anti-inflammatory factors (e.g., IL10, IL1-ra, TGFβ, IL4). Other therapeutic polypeptides for ocular disorders include, but are not limited to, Myo7a, ABCA4, REP1, GUCY2D, PDE6C, RS1, RPGRIP, Lpcatl, AIPL1, RDH12, CHM. In some embodiments, the encoded polypeptide is a human variant of the polypeptide.

Examples of target genes associated with the retinal disorders include tyrosine kinase, endothelial (TEK); complement factor B (CFB); hypoxia-inducible factor 1, α subunit (HIF1A); HtrA senne peptidase 1 (HTRA1); platelet-derived growth factor receptor β (PDGFRB); chemokine, CXC motif, receptor 4 (CXCR4); insulin-like growth factor I receptor (IGFIR); angiopoietin 2 (ANGPT2); v-fos FBJ murine osteosarcoma viral oncogene homolog (FOS); cathepsin L1, transcript variant 1 (CTSLI); cathepsin L1, transcript variant 2 (CTSL2); intracellular adhesion molecule 1 (ICAMI); insulin-like growth factor I (IGF1); integrin a5 (ITGA5); integrin β1 (ITGB1); nuclear factor kappa-B, subunit 1 (NFκB1); nuclear factor kappa-B, subunit 2 (NFκB2); chemokine, CXC motif, ligand 12 (CXCL12); tumor necrosis factor-alpha-converting enzyme (TACE); tumor necrosis factor receptor 1 (TNFR1); vascular endothelial growth factor (VEGF); vascular endothelial growth factor receptor 1 (VEGFR1); and kinase insert domain receptor (KDR).

Examples of target genes associated with glaucoma include carbonic anhydrase II (CA2); carbonic anhydrase IV (CA4); carbonic anhydrase XII (CA12); β1 andrenergic receptor (ADBR1); 32 andrenergic receptor (ADBR2); acetylcholinesterase (ACHE); Na⁺/K⁺-ATPase; solute carrier family 12 (sodium/potassium/chloride transporters), member 1 (SLC12A1); solute carrier family 12 (sodium/potassium/chloride transporters), member 2 (SLC12A2); connective tissue growth factor (CTGF); serum amyloid A (SAA); secreted frizzled-related protein 1 (sFRP1); gremlin (GREMI); lysyl oxidase (LOX); c-Maf; rho-associated coiled-coil-containing protein kinase 1 (ROCK1); rho-associated coiled-coil-containing protein kinase 2 (ROCK2); plasminogen activator inhibitor 1 (PAI-1); endothelial differentiation, sphingolipid G-protein-coupled receptor, 3 (Edg3R); myocilin (MYOC); NADPH oxidase 4 (NOX4); Protein Kinase Cδ (PKC6); Aquaporin 1 (AQP1); Aquaporin 4 (AQP4); members of the complement cascade; ATPase, H+transporting, lysosomal V1 subunit A (ATP6VIA); gap junction protein α-1 (GJA1); formyl peptide receptor 1 (FPR1); formyl peptide receptor-like 1 (FPRL1); interleukin 8 (IL8); nuclear factor kappa-B, subunit 1 (NFκB1); nuclear factor kappa-B, subunit 2 (NFκB2); presenilin 1 (PSEN1); tumor necrosis factor-alpha-converting enzyme (TACE); transforming growth factor β2 (TGFB2); transient receptor potential cation channel, subfamily V, member 1 (TRPV1); chloride channel 3 (CLCN3); gap junction protein α5 (GJA5); tumor necrosis factor receptor 1 (TNFR1); and chitinase 3-like 2 (CHI3L2).

Examples of target genes associated with ocular inflammation include tumor necrosis factor receptor superfamily, member 1A (TNFRSFlA); phosphodiesterase 4D, cAMP-specific (PDE4D); histamine receptor H1 (HRH1); spleen tyrosine kinase (SYK); interkeukin 1β (IL1B); nuclear factor kappa-B, subunit 1 (NFκB1); nuclear factor kappa-B, subunit 2 (NFκB2); and tumor necrosis factor-alpha-converting enzyme (TACE).

Such target genes are described, for example, in U.S. patent applications having Publication Nos. 20060166919, 20060172961, 20060172963, 20060172965, 20060223773, 20070149473, and 20070155690, the disclosures of which are incorporated by reference in their entirety.

### Denatured Therapeutic Nucleic Acids

Aspects of the present disclosure further provide pharmaceutical compositions comprising lipid particles (e.g., pharmaceutical compositions), comprising a lipid nanoparticle (LNP) and a denatured therapeutic nucleic acid (TNA), where TNA is as defined above.

The term “denatured therapeutic nucleic acid” refers to a partially or fully TNA where the conformation has changed from the standard B-form structure. The conformational changes may include changes in the secondary structure (i.e., base pair interactions within a single nucleic acid molecule) and/or changes in the tertiary structure (i.e., double helix structure). Without being bound by theory, it was thought that TNA treated with an alcohol/water solution or pure alcohol solvent results in the denaturation of the nucleic acid to a conformation that enhances encapsulation efficiency by LNP and produces LNP formulations having a smaller diameter size (i.e., smaller than 75 nm, for example, the mean size of about 68 to 74 nm in diameter). All LNP mean diameter sizes and size ranges described herein apply to LNPs containing a denatured TNA. In one embodiment, the denatured TNA is a mRNA. In one embodiment, the denatured TNA is a closed ended DNA (ceDNA).

When DNA is in an aqueous environment, it has a B-form structure with 10.4 base pairs in each complete helical turn. If this aqueous environment is gradually changed by adding a moderately less polar alcohol such as methanol, the twist of the helix relaxes, whereby the DNA changes smoothly into a form with only 10.2 base pairs per helical turn, as visualized by circular dichroism (CD) spectroscopy. In one embodiment, the denatured TNA in a pharmaceutical composition provided herein has a 10.2-form structure.

In contrast to this behavior, if the water is replaced with a slightly less polar alcohol such as ethanol, the same kind of conformational change will occur only until about 65% of the water is replaced with ethanol. At this point, the DNA abruptly changes to the A-form structure which has a more tightly-twisted helix containing 11 base pairs per helical turn, as visualized by CD. In one embodiment, the denatured TNA in a pharmaceutical composition provided herein has an A-form structure.

According to some embodiments, the denatured TNA in a pharmaceutical composition provided herein has a rod-like structure when visualized under transmission electron microscopy (TEM). According to some embodiments, the denatured TNA in a pharmaceutical composition provided herein has a circular-like structure when visualized under transmission electron microscopy (TEM). Comparatively, TNA that has not been denatured has a strand-like structure.

IV. Closed-ended DNA (ceDNA) Vectors

Embodiments of the disclosure are based on methods and compositions comprising closed-ended linear duplexed (ceDNA) vectors that can express a transgene (e.g., a therapeutic nucleic acid (TNA)). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

ceDNA vectors preferably have a linear and continuous structure rather than a non-continuous structure. The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, it is likely to remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

Provided herein are non-viral, capsid-free ceDNA molecules with covalently closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (e.g., a transgene, in particular a therapeutic transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g., AAV ITR); and at least one of the ITRs comprises a functional terminal resolution site (trs) and a Rep binding site. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR. In one embodiment, the first ITR (5′ ITR) and the second ITR (3′ ITR) are asymmetric with respect to each other -that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In one embodiment, the first ITR and the second ITR are both modified but are different sequences, or have different modifications, or are not identical modified ITRs, and have different 3D spatial configurations. Stated differently, a ceDNA vector with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other.

In one embodiment, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other--that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In one embodiment, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be are wild type (WT-ITRs) as described herein. That is, both ITRs have a wild type sequence, but do not necessarily have to be WT-ITRs from the same AAV serotype. In one embodiment, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In one embodiment, a ceDNA vector described herein comprising the expression cassette with a transgene which is a therapeutic nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment, an expression cassette is located between two ITRs comprised in the following order with one or more of: a promoter operably linked to a transgene, a posttranscriptional regulatory element, and a polyadenylation and termination signal. In one embodiment, the promoter is regulatable--inducible or repressible. The promoter can be any sequence that facilitates the transcription of the transgene. In one embodiment the promoter is a CAG promoter, or variation thereof. The posttranscriptional regulatory element is a sequence that modulates expression of the transgene, as a non-limiting example, any sequence that creates a tertiary structure that enhances expression of the transgene which is a therapeutic nucleic acid sequence.

In one embodiment, the posttranscriptional regulatory element comprises WPRE. In one embodiment, the polyadenylation and termination signal comprise BGHpolyA. Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

In one embodiment, the expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a therapeutic nucleic acid sequence in the range of 500 to 50,000 nucleotides in length. In one embodiment, the expression cassette can comprise a transgene which is a therapeutic nucleic acid sequence in the range of 500 to 75,000 nucleotides in length. In one embodiment, the expression cassette can comprise a transgene which is a therapeutic nucleic acid sequence in the range of 500 to 10,000 nucleotides in length. In one embodiment, the expression cassette can comprise a transgene which is a therapeutic nucleic acid sequence in the range of 1000 to 10,000 nucleotides in length. In one embodiment, the expression cassette can comprise a transgene which is a therapeutic nucleic acid sequence in the range of 500 to 5,000 nucleotides in length. The ceDNA vectors do not have the size limitations of encapsidated AAV vectors, and thus enable delivery of a large-size expression cassette to the host. In one embodiment, the ceDNA vector is devoid of prokaryote-specific methylation.

In one embodiment, the rigid therapeutic nucleic acid can be a plasmid.

In one embodiment, the ceDNA vectors disclosed herein are used for therapeutic purposes (e.g., for medical, diagnostic, or veterinary uses) or immunogenic polypeptides.

The expression cassette can comprise any transgene which is a therapeutic nucleic acid sequence. In certain embodiments, the ceDNA vector comprises any gene of interest in the subject, which includes one or more polypeptides, peptides, ribozymes, peptide nucleic acids, siRNAs, RNAis, antisense oligonucleotides, antisense polynucleotides, antibodies, antigen binding fragments, or any combination thereof.

In one embodiment, the ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. In one embodiment, the exogenous sequence such as a donor sequence can encode a gene product that can function to correct the expression of a defective gene or transcript. In one embodiment, the expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., inisteriR)). In one embodiment, expression cassettes can include an exogenous sequence that encodes a reporter protein to be used for experimental or diagnostic purposes, such as b-lactamase, b-galactosidase (LacZ), alkaline phosphatase, thymidine kinase, green fluorescent protein (GFP), chloramphenicol acetyltransferase (CAT), luciferase, and others well known in the art.

Accordingly, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease.

V. Production of a ceDNA Vector

Embodiments of the disclosure are based on compositions comprising a lipid nanoparticle (LNP) and a therapeutic nucleic acid (TNA). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

Methods for the production of a ceDNA vector as described herein comprising an asymmetrical ITR pair or symmetrical ITR pair as defined herein is described in section IV of PCT/US 18/49996 filed Sep. 7, 2018, which is incorporated herein in its entirety by reference. As described herein, the ceDNA vector can be obtained, for example, by the process comprising the steps of: a) incubating a population of host cells (e.g., insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell.

The following is provided as a non-limiting example.

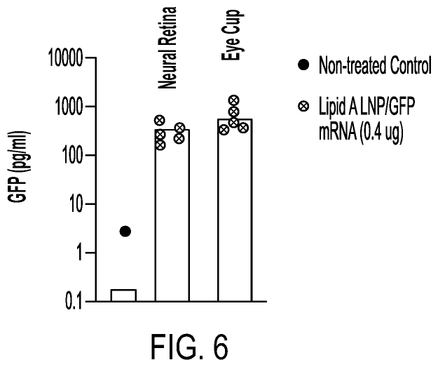

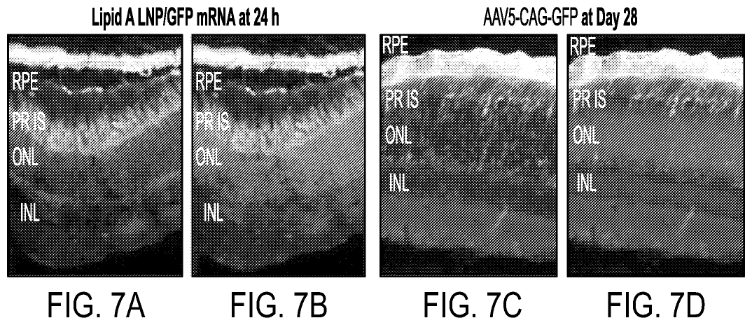

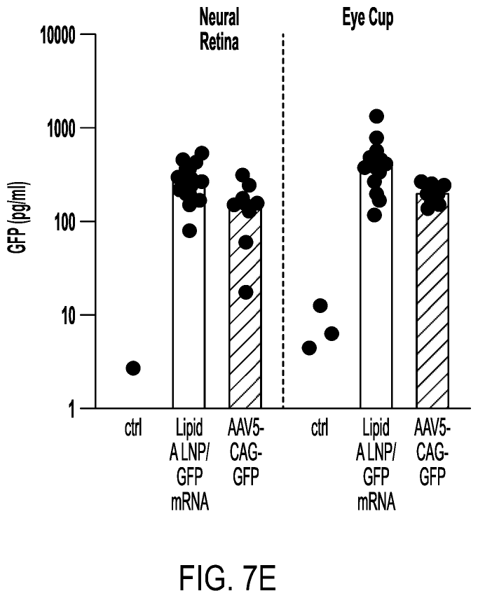

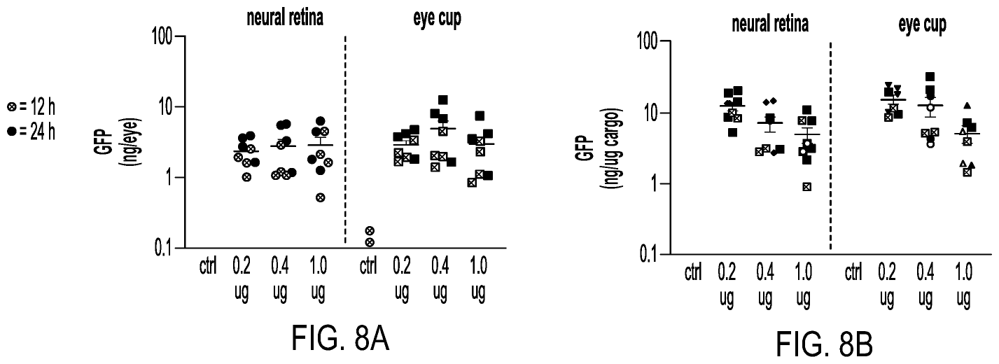

According to some embodiments, synthetic ceDNA is produced via excision from a double-stranded DNA molecule. Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector comprises additional components to regulate expression of the transgene, for example, regulatory switches, to regulate the expression of the transgene, or a kill switch, which can kill a cell comprising the vector.

A molecular regulatory switch is one which generates a measurable change in state in response to a signal. Such regulatory switches can be usefully combined with the ceDNA vectors described herein to control the output of expression of the transgene. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA vector in a controllable and regulatable fashion. In some embodiments, the switch can include a “kill switch” that can instruct the cell comprising the synthetic ceDNA vector to undergo cell programmed death once the switch is activated. Exemplary regulatory switches encompassed for use in a ceDNA vector can be used to regulate the expression of a transgene, and are more fully discussed in International application PCT/US18/49996, which is incorporated herein in its entirety by reference and described herein.

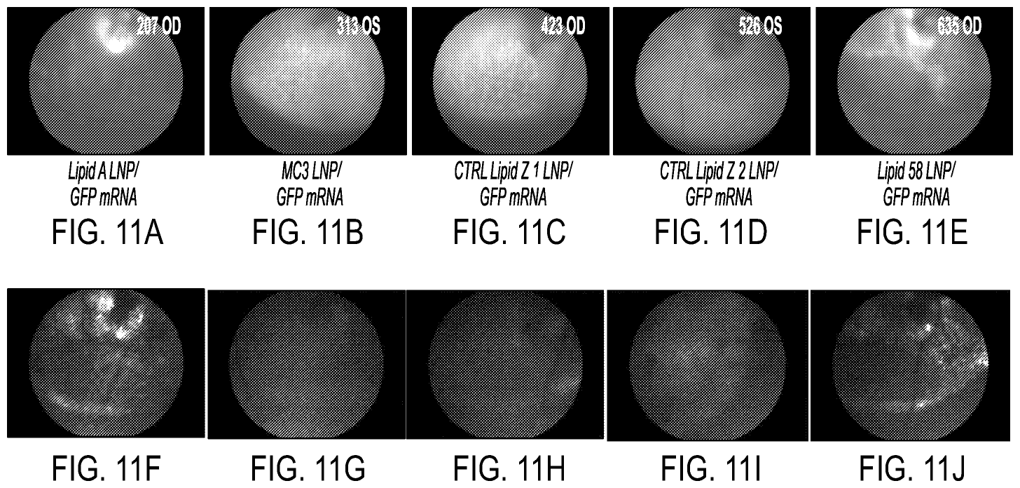

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122, incorporated by reference in its entirety herein, shows an exemplary method of ligating a 5′ ITR oligonucleotide and a 3′ ITR oligonucleotide to a double stranded polynucleotide comprising an expression cassette.

An exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

In yet another aspect, the invention provides for host cell lines that have stably integrated the DNA vector polynucleotide expression template (ceDNA template) described herein, into their own genome for use in production of the non-viral DNA vector. Methods for producing such cell lines are described in Lee, L. et al. (2013) Plos One 8(8): e69879, which is herein incorporated by reference in its entirety. For example, the Rep protein is added to host cells at an MOI of 3. In one embodiment, the host cell line is an invertebrate cell line, preferably insect Sf9 cells. When the host cell line is a mammalian cell line, preferably 293 cells the cell lines can have polynucleotide vector template stably integrated, and a second vector, such as herpes virus can be used to introduce Rep protein into cells, allowing for the excision and amplification of ceDNA in the presence of Rep.

Any promoter can be operably linked to the heterologous nucleic acid (e.g. reporter nucleic acid or therapeutic transgene) of the vector polynucleotide. The expression cassette can contain a synthetic regulatory element, such as CAG promoter. The CAG promoter comprises (i) the cytomegalovirus (CMV) early enhancer element, (ii) the promoter, the first exon and the first intron of the chicken beta actin gene, and (ii) the splice acceptor of the rabbit beta globin gene. Alternatively, expression cassette can contain an Alpha-i-antitrypsin (AAT) promoter, a liver specific (LP1) promoter, or Human elongation factor-1 alpha (EF1-a) promoter. In some embodiments, the expression cassette includes one or more constitutive promoters, for example, the retroviral Rous sarcoma virus (RSV) LTR promoter (optionally with the RSV enhancer), cytomegalovirus (CMV) immediate early promoter (optionally with the CMV enhancer). Alternatively, an inducible or repressible promoter, a native promoter for a transgene, a tissue-specific promoter, or various promoters known in the art can be used. Suitable transgenes for gene therapy are well known to those of skill in the art.

The capsid-free ceDNA vectors can also be produced from vector polynucleotide expression constructs that further comprise cis-regulatory elements, or combination of cis regulatory elements, a non-limiting example include a woodchuck hepatitis virus posttranscriptional regulatory element (WPRE) and BGH polyA, or e.g., beta-globin polyA. Other posttranscriptional processing elements include, e.g., the thymidine kinase gene of herpes simplex virus, or hepatitis B virus (HBV). The expression cassettes can include any poly-adenylation sequence known in the art or a variation thereof, such as a naturally occurring isolated from bovine BGHpA or a virus SV40 pA, or synthetic. Some expression cassettes can also include SV40 late polyA signal upstream enhancer (USE) sequence. The USE can be used in combination with SV40 pA or heterologous poly-A signal.

The time for harvesting and collecting DNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce DNA-vectors, but before the majority of cells start to die because of the viral toxicity. The DNA-vectors can be isolated using plasmid purification kits such as Qiagen Endo-Free Plasmid kits. Other methods developed for plasmid isolation can be also adapted for DNA-vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

In one embodiment, the capsid free non-viral DNA vector comprises or is obtained from a plasmid comprising a polynucleotide template comprising in this order: a first adeno-associated virus (AAV) inverted terminal repeat (ITR), a nucleotide sequence of interest (for example an expression cassette of an exogenous DNA) and a modified AAV ITR, wherein said template nucleic acid molecule is devoid of AAV capsid protein coding. In a further embodiment, the nucleic acid template of the invention is devoid of viral capsid protein coding sequences (i.e., it is devoid of AAV capsid genes but also of capsid genes of other viruses). In addition, in a particular embodiment, the template nucleic acid molecule is also devoid of AAV Rep protein coding sequences. Accordingly, in a preferred embodiment, the nucleic acid molecule of the invention is devoid of both functional AAV cap and AAV rep genes.

In one embodiment, ceDNA can include an ITR structure that is mutated with respect to the wild type AAV2 ITR disclosed herein, but still retains an operable RBE, TRS and RBE′ portion.

ceDNA Plasmid

A ceDNA-plasmid is a plasmid used for later production of a ceDNA vector. In one embodiment, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one embodiment, a ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In one embodiment, the ceDNA-plasmid system is devoid of viral capsid protein coding sequences (i.e. it is devoid of AAV capsid genes but also of capsid genes of other viruses). In one embodiment, the ceDNA-plasmid is also devoid of AAV Rep protein coding sequences. In one embodiment, ceDNA-plasmid is devoid of functional AAV cap and AAV rep genes GG-3′ for AAV2) plus a variable palindromic sequence allowing for hairpin formation. In one embodiment, a ceDNA-plasmid of the present disclosure can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV 10, AAV 11, AAV 12, AAVrh8, AAVrhlO, AAV-DJ, and AAV-DJ8 genome, e.g., NCBI: NC 002077; NC 001401; NC001729; NC001829; NC006152; NC 006260; NC 006261; Kotin and Smith, The Springer Index of Viruses, available at the URL maintained by Springer. In one embodiment, the ceDNA-plasmid backbone is derived from the AAV2 genome. In one embodiment, the ceDNA-plasmid backbone is a synthetic backbone genetically engineered to include at its 5′ and 3′ ITRs derived from one of these AAV genomes.

In one embodiment, a ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like.

### VI. Preparation of Lipid Particles

Lipid particles (e.g., lipid nanoparticles) can form spontaneously upon mixing of ceDNA and the lipid(s). Depending on the desired particle size distribution, the resultant nanoparticle mixture can be extruded through a membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. In one embodiment, the lipid nanoparticles are formed as described in Example 3 described in U.S. Provisional Application No. 63/194,620.

Generally, lipid particles (e.g., lipid nanoparticles) can be formed by any method known in the art. For example, the lipid particles (e.g., lipid nanoparticles) can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, lipid particles (e.g., lipid nanoparticles) can be prepared using a continuous mixing method, a direct dilution process, or an in-line dilution process. The processes and apparatuses for apparatuses for preparing lipid nanoparticles using direct dilution and in-line dilution processes are described in US2007/0042031, the content of which is incorporated herein by reference in its entirety. The processes and apparatuses for preparing lipid nanoparticles using step-wise dilution processes are described in US2004/0142025, the content of which is incorporated herein by reference in its entirety.

According to some embodiments, the disclosure provides for an LNP comprising a DNA vector, including a ceDNA vector as described herein and an ionizable lipid. For example, a lipid nanoparticle formulation that is made and loaded with therapeutic nucleic acid like ceDNA obtained by the process as disclosed in International Patent Application No. PCT/US2018/050042, filed on Sep. 7, 2018, which is incorporated by reference in its entirety herein.

In one embodiment, the lipid particles (e.g., lipid nanoparticles) can be prepared by an impinging jet process. Generally, the particles are formed by mixing lipids dissolved in alcohol (e.g., ethanol) with ceDNA dissolved in a buffer, e.g., a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA can be about 45-55% lipid and about 65-45% ceDNA.

The lipid solution can contain a cationic lipid (e.g., an ionizable cationic lipid), a non-cationic lipid (e.g., a phospholipid, such as DSPC, DOPE, and DOPC), PEG or PEG conjugated molecule (e.g., PEG-lipid), and a sterol (e.g., cholesterol) at a total lipid concentration of 5-30 mg/mL, more likely 5-15 mg/mL, most likely 9-12 mg/mL in an alcohol, e.g., in ethanol. In the lipid solution, mol ratio of the lipids can range from about 25-98% for the cationic lipid, preferably about 35-65%; about 0-15% for the non-ionic lipid, preferably about 0-12%; about 0-15% for the PEG or PEG conjugated lipid molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 mL/min. The tube ID can have a range from 0.25 to 1.0 mm and a total flow rate from 10-600 mL/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30 mins to 2 hours. The temperature during incubating can be in the range of 15-40° C. or 30-40° C. After incubating the solution is filtered through a filter, such as a 0.8 μm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about pH 7.0, about pH 7.1, about pH 7.2, about pH 7.3, or about pH 7.4.

The ultrafiltration process can use a tangential flow filtration format (TFF) using a membrane nominal molecular weight cutoff range from 30-500 kD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1-3 mg/mL. Following concentration, the LNPs solution is diafiltered against the final buffer for 10-20 volumes to remove the alcohol and perform buffer exchange. The material can then be concentrated an additional 1-3-fold. The concentrated LNP solution can be sterile filtered.

### VII. Pharmaceutical Compositions and Formulations

Provided herein is a pharmaceutical composition comprising a lipid nanoparticle (LNP), a therapeutic nucleic acid (TNA) and at least one pharmaceutically acceptable excipient, wherein the LNP comprises at least one lipid, and wherein the LNP is capable of delivering the TNA to a retinal cell. According to some embodiments, such excipients include any pharmaceutical agent suitable for direct delivery to the eye.

In one embodiment, the lipid particles (e.g., lipid nanoparticles) are substantially non-toxic to a subject, e.g., to a mammal such as a human.

In some embodiments, the LNP comprises a lipid selected from the group consisting of a cationic lipid, a sterol or a derivative thereof, a non-cationic lipid, and at least one PEGylated lipid, as described herein. In some embodiments, the LNP comprises a cationic lipid. In some embodiments, the LNP comprises a sterol or derivative thereof. In some embodiments, the LNP comprises a non-cationic lipid. In some embodiments, the LNP comprises at least one PEGylated lipid.

In one embodiment, the lipid particles (e.g., lipid nanoparticles) may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

According to some embodiments, the TNA (e.g., ceDNA) is encapsulated in the lipid. In one embodiment, the TNA can be fully encapsulated in the lipid position of the lipid particle (e.g., lipid nanoparticle), thereby protecting it from degradation by a nuclease, e.g., in an aqueous solution. In one embodiment, the TNA in the lipid particle (e.g., lipid nanoparticle) is not substantially degraded after exposure of the lipid particle (e.g., lipid nanoparticle) to a nuclease at 37° C. for at least about 20, 30, 45, or 60 minutes. In some embodiments, the TNA in the lipid particle (e.g., lipid nanoparticle) is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In one embodiment, encapsulation of TNA (e.g., ceDNA) in lipid particles (e.g., lipid nanoparticles) can be determined by performing a membrane-impermeable fluorescent dye exclusion assay, which uses a dye that has enhanced fluorescence when associated with nucleic acid, for example, an OLIGREEN® assay or PICOGREEN® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated TNA (e.g., ceDNA), allowing it to interact with the membrane-impermeable dye. Encapsulation of ceDNA can be calculated as E=(Io-I)/Io, where I and Io refer to the fluorescence intensities before and after the addition of detergent.

According to some embodiments, the TNA is selected from the group consisting of minigenes, plasmids, minicircles, small interfering RNA (siRNA), microRNA (miRNA), antisense oligonucleotides (ASO), ribozymes, closed-ended (ceDNA), ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, gRNA, DNA viral vectors, viral RNA vector, non-viral vector and any combination thereof.

According to some embodiments, the TNA is ceDNA. According to some embodiments, the ceDNA is linear duplex DNA. According to some embodiments, the TNA is mRNA. According to some embodiments, the TNA is siRNA. According to some embodiments, the TNA is a plasmid.

According to some embodiments, the LNP has a total lipid to TNA ratio of about 10:1 to about 40:1.

According to some embodiments, the LNP comprises a cationic lipid, wherein the cationic lipid is present at a molar percentage of about 30% to about 80%. According to some embodiments, the LNP comprises a cationic lipid, wherein the cationic lipid is present at a molar percentage of about 30% to about 70%. According to some embodiments, the LNP comprises a cationic lipid, wherein the cationic lipid is present at a molar percentage of about 30% to about 60%. According to some embodiments, the LNP comprises a cationic lipid, wherein the cationic lipid is present at a molar percentage of about 30% to about 50%. According to some embodiments, the LNP comprises a cationic lipid, wherein the cationic lipid is present at a molar percentage of about 30% to about 40%. According to some embodiments, the LNP comprises a cationic lipid, wherein the cationic lipid is present at a molar percentage of about 40% to about 80%. According to some embodiments, the LNP comprises a cationic lipid, wherein the cationic lipid is present at a molar percentage of about 30% to about 70%. According to some embodiments, the LNP comprises a cationic lipid, wherein the cationic lipid is present at a molar percentage of about 40% to about 60%. According to some embodiments, the LNP comprises a cationic lipid, wherein the cationic lipid is present at a molar percentage of about 40% to about 50%. According to some embodiments, the LNP comprises a cationic lipid, wherein the cationic lipid is present at a molar percentage of about 50% to about 80%. According to some embodiments, the LNP comprises a cationic lipid, wherein the cationic lipid is present at a molar percentage of about 50% to about 70%. According to some embodiments, the LNP comprises a cationic lipid, wherein the cationic lipid is present at a molar percentage of about 50% to about 60%. According to some embodiments, the LNP comprises a cationic lipid, wherein the cationic lipid is present at a molar percentage of about 60% to about 80%. According to some embodiments, the LNP comprises a cationic lipid, wherein the cationic lipid is present at a molar percentage of about 70% to about 80%.

According to some embodiments, the LNP comprises a sterol, wherein the sterol is present at a molar percentage of about 20% to about 50%. According to some embodiments, the LNP comprises a sterol, wherein the sterol is present at a molar percentage of about 30% to about 50%. According to some embodiments, the LNP comprises a sterol, wherein the sterol is present at a molar percentage of about 40% to about 50%. According to some embodiments, the LNP comprises a sterol, wherein the sterol is present at a molar percentage of about 20% to about 40%. According to some embodiments, the LNP comprises a sterol, wherein the sterol is present at a molar percentage of about 30% to about 40%.

According to some embodiments, the LNP comprises a non-cationic lipid, wherein the non-cationic lipid is present at a molar percentage of about 2% to about 20%. According to some embodiments, the LNP comprises a non-cationic lipid, wherein the non-cationic lipid is present at a molar percentage of about 5% to about 20%. According to some embodiments, the LNP comprises a non-cationic lipid, wherein the non-cationic lipid is present at a molar percentage of about 10% to about 20%. According to some embodiments, the LNP comprises a non-cationic lipid, wherein the non-cationic lipid is present at a molar percentage of about 15% to about 20%. According to some embodiments, the LNP comprises a non-cationic lipid, wherein the non-cationic lipid is present at a molar percentage of about 10% to about 20%. According to some embodiments, the LNP comprises a non-cationic lipid, wherein the non-cationic lipid is present at a molar percentage of about 10% to about 15%.

According to some embodiments, the LNP comprises at least one PEGylated lipid, wherein the PEGylated lipid is present at a molar percentage of about 2.1% to about 10%. According to some embodiments, the LNP comprises at least one PEGylated lipid, wherein the PEGylated lipid is present at a molar percentage of about 5% to about 10%. According to some embodiments, the LNP comprises at least one PEGylated lipid, wherein the PEGylated lipid is present at a molar percentage of about 7% to about 10%. According to some embodiments, the LNP comprises at least one PEGylated lipid, wherein the PEGylated lipid is present at a molar percentage of about 2.10% to about 8%. According to some embodiments, the LNP comprises at least one PEGylated lipid, wherein the PEGylated lipid is present at a molar percentage of about 2.1% to about 5%. According to some embodiments, the LNP comprises at least one PEGylated lipid, wherein the PEGylated lipid is present at a molar percentage of about 5% to about 8%.

According to some embodiments, the LNP comprises at least one PEGylated lipid, wherein the PEGylated lipid is present at a molar percentage of about 1% to about 2%. According to some embodiments, the LNP comprises at least one PEGylated lipid, wherein the PEGylated lipid is present at a molar percentage of about 1.2% to about 2%. According to some embodiments, the LNP comprises at least one PEGylated lipid, wherein the PEGylated lipid is present at a molar percentage of about 1.5% to about 2%. According to some embodiments, the LNP comprises at least one PEGylated lipid, wherein the PEGylated lipid is present at a molar percentage of about 1.75% to about 2%. According to some embodiments, the LNP comprises at least one PEGylated lipid, wherein the PEGylated lipid is present at a molar percentage of about 1% to about 1.5%. According to some embodiments, the LNP comprises at least one PEGylated lipid, wherein the PEGylated lipid is present at a molar percentage of about 1.25% to about 1.5%. According to some embodiments, the LNP comprises at least one PEGylated lipid, wherein the PEGylated lipid is present at a molar percentage of about 1.5% to about 1.75%.

Depending on the intended use of the lipid particles (e.g., lipid nanoparticles), the proportions of the components can be varied and the delivery efficiency of a particular formulation can be measured using, for example, an endosomal release parameter (ERP) assay.

According to some embodiments, the pharmaceutical compositions may further comprise dexamethasone palmitate.

According to some embodiments of the pharmaceutical compositions described herein, the LNP comprises Lipid A, DOPC, cholesterol and DMG-PEG. According to some embodiments of the pharmaceutical compositions described herein, LNP comprises Lipid A, DOPC, cholesterol, DMG-PEG, and DSPE-PEG. According to some embodiments of the pharmaceutical compositions described herein, the LNP comprises Lipid A, DOPE, cholesterol and DMG-PEG. According to some embodiments of the pharmaceutical compositions described herein, the LNP comprises Lipid A, DOPE, cholesterol, DMG-PEG, and DSPE-PEG. According to some embodiments of the pharmaceutical compositions described herein, the LNP comprises Lipid A, DSPC, cholesterol and DMG-PEG. According to some embodiments of the pharmaceutical compositions described herein, the e LNP comprises Lipid A, DSPC, cholesterol, DMG-PEG, and DSPE-PEG. According to some embodiments of the pharmaceutical compositions described herein, the LNP comprises Lipid A, DOPC, beta-sitosterol and DMG-PEG. According to some embodiments of the pharmaceutical compositions described herein, the LNP comprises Lipid A, DOPC, beta-sitosterol, DMG-PEG, and DSPE-PEG. According to some embodiments of the pharmaceutical compositions described herein, the LNP comprises Lipid A, DOPE, beta-sitosterol and DMG-PEG. According to some embodiments of the pharmaceutical compositions described herein, the LNP comprises Lipid A, DOPE, beta-sitosterol, DMG-PEG, and DSPE-PEG. According to some embodiments of the pharmaceutical compositions described herein, the LNP comprises Lipid A, DSPC, beta-sitosterol and DMG-PEG. According to some embodiments of the pharmaceutical compositions described herein, the LNP comprises Lipid A, DSPC, beta-sitosterol, DMG-PEG, and DSPE-PEG.

According to some embodiments of any of the aspects and embodiments herein, the DMG-PEG is DMG-PEG2000.

According to some embodiments of any of the aspects and embodiments herein, the DSPE-PEG is DSPE-PEG2000.

According to some embodiments of any of the aspects and embodiments herein, the DSPE-PEG is DSPE-PEG5000.

According to some embodiments of any of the aspects and embodiments herein, the LNP comprises Lipid A, DOPC, sterol, DMG-PEG and DSPE-PEG at molar ratios of about 51: 7.3: 38.3:2.9:0.5.

According to some aspects, the disclosure provides a pharmaceutical composition comprising a LNP, a therapeutic messenger RNA (mRNA), and at least one pharmaceutically acceptable excipient; wherein the LNP is capable of delivering the mRNA to a retinal cell. According to some embodiments, the LNP is capable of delivering the mRNA to a photoreceptor (PR) cell. According to some embodiments, the LNP is capable of delivering the mRNA to a retina pigment epithelium (RPE) cell. According to some embodiments, the LNP is capable of being internalized into the PR cell and/or the RPE cell. According to some embodiments, the mRNA expression is evenly distributed in the PR cell and the RPE cell.

According to some embodiments, the LNP is capable of delivering the mRNA to a retinal cell without resulting in retinal degradation or thinning of the outer nuclear layer (ONL), compared to a suitable control.

According to some embodiments, the mRNA is encapsulated in the LNP.

According to some embodiments, the LNP further comprises a lipid selected from the group consisting of a sterol or a derivative thereof, a non-cationic lipid, and at least one PEGylated lipid. According to further embodiments, the sterol or a derivative thereof is a cholesterol. According to other embodiments, the sterol or a derivative thereof is beta-sitosterol. According to some embodiments, the non-cationic lipid is selected from the group consisting of dioleoylphosphatidylcholine (DOPC), distearoylphosphatidylcholine (DSPC), and dioleoyl-phosphatidylethanolamine (DOPE). According to some embodiments, the PEGylated lipid is DMG-PEG, DSPE-PEG, DSPE-PEG-OH, DSG-PEG, or a combination thereof. According to some embodiments, the at least one PEGylated lipid is DMG-PEG2000, DSPE-PEG2000, DSPE-PEG2000-OH, DSG-PEG, or a combination thereof. According to some embodiments, the LNP comprises Lipid A, DOPC, cholesterol and DMG-PEG; or Lipid A, DOPE, cholesterol and DMG-PEG; or

Lipid A, DSPC, cholesterol and DMG-PEG; or Lipid A, DSPC, cholesterol, DMG-PEG, and DSPE-PEG; Lipid A, DOPC, beta-sitosterol and DMG-PEG; or Lipid A, DOPC, beta-sitosterol, DMG-PEG, and DSPE-PEG; or Lipid A, DOPE, beta-sitosterol and DMG-PEG; or Lipid A, DOPE, beta-sitosterol, DMG-PEG, and DSPE-PEG; or Lipid A, DSPC, beta-sitosterol and DMG-PEG; or Lipid A, DSPC, beta-sitosterol, DMG-PEG, and DSPE-PEG.

According to some embodiments, the DMG-PEG is DMG-PEG2000.

According to some embodiments, the DSPE-PEG is DSPE-PEG2000.

According to some embodiments, the DSPE-PEG is DSPE-PEG5000.

According to some embodiments, the LNP comprises Lipid A, DOPC, sterol, DMG-PEG and DSPE-PEG at molar ratios of about 51:7.3:38.3:2.9:0.5.

In one embodiment, the lipid particle formulation is an aqueous solution. In one embodiment, the lipid particle (e.g., lipid nanoparticle) formulation is a lyophilized powder.

According to some aspects, the disclosure provides for a lipid particle formulation further comprising one or more pharmaceutical excipients. In one embodiment, the lipid particle (e.g., lipid nanoparticle) formulation further comprises sucrose, tris, trehalose and/or glycine.

Pharmaceutical compositions for therapeutic purposes can be formulated as a solution, microemulsion, dispersion, liposomes, or other ordered structure suitable for high TNA (e.g., ceDNA) vector concentration. Sterile injectable solutions can be prepared by incorporating the TNA (e.g., ceDNA) vector compound in the required amount in an appropriate buffer (e.g., pharmaceutically acceptable excipient) with one or a combination of ingredients enumerated above, as required, followed by filtered sterilization.

Pharmaceutical compositions for therapeutic purposes typically must be sterile and stable under the conditions of manufacture and storage. The composition can be formulated as a solution, microemulsion, dispersion, liposomes, or other ordered structure suitable to high TNA (e.g., ceDNA) vector concentration. Sterile injectable solutions can be prepared by incorporating the ceDNA vector compound in the required amount in an appropriate buffer with one or a combination of ingredients enumerated above, as required, followed by filtered sterilization.

In one embodiment, lipid particles (e.g., lipid nanoparticles) are solid core particles that possess at least one lipid bilayer. In one embodiment, the lipid particles (e.g., lipid nanoparticles) have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the lipid particles (e.g., lipid nanoparticles) can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid particles (lamellar vs. non-lamellar) can readily be assessed and characterized using, e.g., Cryo-TEM analysis as described in US2010/0130588, the content of which is incorporated herein by reference in its entirety.

In one embodiment, the lipid particles (e.g., lipid nanoparticles) having a non-lamellar morphology are electron dense.

In one embodiment, the disclosure provides for a lipid particle (e.g., lipid nanoparticle) that is either unilamellar or multilamellar in structure. In some aspects, the disclosure provides for a lipid particle (e.g., lipid nanoparticle) formulation that comprises multi-vesicular particles and/or foam-based particles. By controlling the composition and concentration of the lipid components, one can control the rate at which the lipid conjugate exchanges out of the lipid particle and, in turn, the rate at which the lipid particle (e.g., lipid nanoparticle) becomes fusogenic. In addition, other variables including, for example, pH, temperature, or ionic strength, can be used to vary and/or control the rate at which the lipid particle (e.g., lipid nanoparticle) becomes fusogenic. Other methods which can be used to control the rate at which the lipid particle (e.g., lipid nanoparticle) becomes fusogenic will be apparent to those of ordinary skill in the art based on this disclosure. It will also be apparent that by controlling the composition and concentration of the lipid conjugate, one can control the lipid particle size.

In one embodiment, the pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which are incorporated by reference in their entireties). In one embodiment, the preferred range of pKa is ˜5 to ˜7. In one embodiment, the pKa of the cationic lipid can be determined in lipid particles (e.g., lipid nanoparticles) using an assay based on fluorescence of 2-(p-toluidino)-6-napthalene sulfonic acid (TNS).

According to some embodiments, for ophthalmic delivery, interfering RNA-ligand conjugates and nanoparticle-ligand conjugates may be combined with ophthalmologically acceptable preservatives, co-solvents, surfactants, viscosity enhancers, penetration enhancers, buffers, sodium chloride, or water to form an aqueous, sterile ophthalmic suspension or solution.

### Unit Dosage

In one embodiment, the pharmaceutical compositions can be presented in unit dosage form. A unit dosage form will typically be adapted to one or more specific routes of administration of the pharmaceutical composition.

In some embodiments, the unit dosage form is adapted for intravenous, intramuscular, or subcutaneous administration. In some embodiments, the unit dosage form is adapted for intrathecal or intracerebroventricular administration. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the pharmaceutical composition is formulated for topical administration.

The amount of active ingredient which can be combined with a carrier material to produce a single dosage form will generally be that amount of the compound which produces a therapeutic effect.

According to some embodiments, the LNP/TNA is for administration at a dose of about 0.03 μg to about 2.0 μg, or about 0.05 μg to about 2.0 μg, about 0.1 μg to about 2.0 μg, about 0.5 μg to about 2.0 μg, about 1.0 μg to about 2.0 μg, about 1.5 μg to about 2.0 μg, about 0.03 μg to about 1.5 μg, about 0.05 μg to about 1.5 μg, about 0.1 μg to about 1.5 μg, about 0.5 μg to about 1.5 μg, about 1.0 μg to about 1.5 μg, about 0.03 μg to about 1.0 μg, about 0.05 μg to about 1.0 μg, about 0.1 μg to about 1.0 μg, about 0.5 μg to about 1.0 μg, about 0.03 μg to about 0.5 μg, about 0.05 μg to about 0.5 μg, about 0.1 μg to about 0.5 μg, about 0.03 μg to about 0.1 μg, about 0.05 μg to about 0.1 μg, or about 0.03 μg to about 0.05 μg.

According to some embodiments, the LNP/TNA is for administration at a dose of about 0.1 μg to about 1.0 μg, or about 0.1 μg to about 0.9 μg, about 0.1 μg to about 0.8 μg, about 0.1 μg to about 0.7 μg, about 0.1 μg to about 0.6 μg, about 0.1 μg to about 0.5 μg, about 0.1 μg to about 0.4 μg, about 0.1 μg to about 0.3 μg or about 0.1 μg to about 0.2 μg.

According to some embodiments, the LNP/TNA is for administration at a dose of about 0.1 μg to about 0.2 μg, for example a dose of about 0.1 μg, 0.11 μg, 0.12 μg, 0.13 μg, 0.14 μg, 0.15 μg, 0.16 μg, 0.17 μg, 0.18 μg, 0.19 μg or about 0.2 μg.

### VIII. Methods of Treatment

The pharmaceutical compositions comprising a lipid nanoparticle (LNP), a therapeutic nucleic acid (TNA) and at least one pharmaceutically acceptable excipient, wherein the LNP comprises at least one lipid, can be used to introduce a nucleic acid sequence (e.g., a TNA) in a retinal cell. In one embodiment, introduction of a nucleic acid sequence in a host cell using the pharmaceutical compositions comprising a LNP and a therapeutic nucleic acid (TNA), as described herein,

Provided herein are methods of treating a disease or disorder in a subject comprising introducing into a target cell in need thereof (for example, a retinal cell) of the subject a therapeutically effective amount of a pharmaceutical composition comprising a lipid nanoparticle (LNP), a therapeutic nucleic acid (TNA) and at least one pharmaceutically acceptable excipient, wherein the LNP comprises at least one lipid, as described herein. While the TNA lipid nanoparticles can be introduced in the presence of a carrier, such a carrier is not required.

In one embodiment, the target cells are in a human subject.

Provided herein in other aspects are methods of treating an ocular disorder in a subject, the method comprising administering to the subject a therapeutically effective amount of a pharmaceutical composition comprising a lipid nanoparticle (LNP), a therapeutic nucleic acid (TNA) and at least one pharmaceutically acceptable excipient, wherein the LNP comprises at least one lipid, as described herein.

According to some embodiments, the ocular disorder comprises retinal degeneration. According to further embodiments, the ocular disorder is a retinal dystrophy.

According to some embodiments, non-limiting examples of ocular disorders that can be treated by the systems and methods of the invention include the following: autosomal recessive severe early-onset retinal degeneration (Leber congenital amaurosis), congenital color blindness, Stargardt's disease, Best's disease, Doin's disease, cone dystrophy, retinitis pigmentosa, X-linked retinoschisis, Usher's syndrome, age-related macular degeneration, atrophic age-related macular degeneration, angiogenic AMD, diabetic macular disease, proliferation Diabetic retinopathy (PDR), cystoid macular edema, central serous retinopathy, retinal detachment, intraocular inflammation, glaucoma, posterior uveitis, total choroidal atrophy, and Leber's hereditary optic atrophy.

According to some embodiments, the ocular disorder is Stargardt macular dystrophy. According to some embodiments, the ocular disorder is Leber congenital amaurosis-10 (LCA10). According to some embodiments, the ocular disorder is Usher syndrome. According to some embodiments, the ocular disorder is wet AMD. According to some embodiments, the subject is a human in need of treatment with LNP encapsulated with TNA.

In the case of diseases that result in progressive degenerative visual function, treating a subject at an early age can not only slow down or halt the progression of the disease, but also improve or prevent the loss of visual function.

Also provided in another aspect are methods of restoring vision in a subject, the method comprising administering to the subject a therapeutically effective amount of a pharmaceutical composition comprising a lipid nanoparticle (LNP), a therapeutic nucleic acid (TNA) and at least one pharmaceutically acceptable excipient, wherein the LNP comprises at least one lipid, as described herein. By restoring vision in a subject is meant that the subject shows improved vision compared to prior to treatment, for example using vision tests. Restoring includes any degree in improvement, including full restoration of vision to perfect or near perfect vision.

According to some embodiments of any of the aspects and embodiment herein, the human subject has a hereditary eye disorder but has not yet shown clinical signs or symptoms.

According to some embodiments, the methods described herein may comprise a first step of diagnosing a subject for an ocular disease, for example a retinal degenerative condition, for example a retinal dystrophy. A diagnostic step may comprise a visual test, for example a pupillary light reflex (PLR) test, visual acuity test (LogMAR), clinical diagnostic tests for example biomicroscopy/slit-lamp ocular/retinal clinical examination; color vision testing, visual field testing, contrast/full field sensitivity; electrodiagnostic tests including for example EGGs, VEPs; imaging, retinal fundus photography, OCT, and adaptive optics scanning laser ophthalmoscope (AOSLO). Other suitable tests are known to persons skilled in the art.

According to other embodiments, the methods of the disclosure may further comprise a monitoring step comprising monitoring the vision of a subject who has been treated with the pharmaceutical compositions described herein, for an improvement in vision. Improvements in vision may be any one or more of the following: increased pupillary light reflex (PLR), increased contrast sensitivity, increased resolution of low or high frequency flicker, and increased detection of moving images. In addition, increased light induced locomotor activity may be improved in animals such as mice. An improvement in vision may be an ability to respond to or detect light at 10¹⁵-10¹³ photon/cm2/s corneal irradiance. An improvement in vision may comprise an ON-sustained, ON-transient, OFF-excitatory, OFF-inhibitory or ON-OFF response. Preferably, monitoring improvement may comprise a method of quantifying the subjects subjective visual experience or an objective measure of light response, for example a pupillary light reflex (PLR) test, LogMAR visual acuity, clinical examination slit-lamp biomicroscopy; color vision testing, visual field testing, contrast/full field sensitivity; electrodiagnostics-ERGs, VEPs; imaging: retinal fundus photography, OCT, adaptive optics scanning laser ophthalmoscope (AOSLO), or maze navigation tasks.

According to some embodiments, a subject may be monitored every 6, 8, 10, 12 or 24 hours, or every 2, 3, 4, 5 days. This may be repeated after 1, 2, 3, 4, 5, 6 months or a year or more.

According to some embodiments of any of the aspects and embodiments herein, the human subject is younger than 30 years of age. In some embodiments, the human subject being treated is younger than 20 years of age. In some embodiments, the human subject being treated is younger than 18 years of age. In some embodiments, the human subject being treated is less than 15 years of age. In some embodiments, the human subject being treated is younger than 14 years of age. In some embodiments, the human subject being treated is younger than 13 years of age. In some embodiments, the human subject being treated is younger than 12 years of age. In some embodiments, the human subject being treated is younger than 10 years of age. In some embodiments, the human subject being treated is less than 8 years of age. In some embodiments, the human subject being treated is younger than 6 years of age.

Provided herein in some aspects are method of delivering a therapeutic nucleic acid (TNA) or increasing the concentration of the TNA in the retina of a subject, the method comprising administering to the subject a therapeutically effective amount of a pharmaceutical composition comprising a lipid nanoparticle (LNP), a therapeutic nucleic acid (TNA) and at least one pharmaceutically acceptable excipient, wherein the LNP comprises at least one lipid, as described herein.

According to some embodiments, when the pharmaceutical composition is administered to the subject, retinal degeneration does not exceed more than 10% of outer nuclear layer (ONL) loss after 7 days. According to some embodiments, when the pharmaceutical composition is administered to the subject, retinal degeneration does not exceed more than 9%, 8%, 7%, 6%, or 5% of outer nuclear layer (ONL) loss after 7 days. According to some embodiments, when the pharmaceutical composition is administered to the subject, retinal degeneration does not exceed more than 10% of outer nuclear layer (ONL) loss after 14 days. According to some embodiments, when the pharmaceutical composition is administered to the subject, retinal degeneration does not exceed more than 9%, 8%, 7%, 6%, or 5% of outer nuclear layer (ONL) loss after 14 days. According to some embodiments, when the pharmaceutical composition is administered to the subject, retinal degeneration does not exceed more than 10% of outer nuclear layer (ONL) loss after 21 days. According to some embodiments, when the pharmaceutical composition is administered to the subject, retinal degeneration does not exceed more than 9%, 8%, 7%, 6%, or 5% of outer nuclear layer (ONL) loss after 12 days.

According to some embodiments, ONL loss is measured by Optical Coherence Tomography (OCT).

### Administration

In one embodiment, the pharmaceutical compositions comprising a LNP and a therapeutic nucleic acid (TNA), as described herein, can be administered to an organism for transduction of cells in vivo. In one embodiment, the pharmaceutical compositions comprising a LNP and a TNA, as describe herein, can be administered to an organism for transduction of cells ex vivo.

According to some embodiments, the LNP/TNA is for administration at a dose of about 0.03 μg to about 2.0 μg, or about 0.05 μg to about 2.0 μg, about 0.1 μg to about 2.0 μg, about 0.5 μg to about 2.0 μg, about 1.0 μg to about 2.0 μg, about 1.5 μg to about 2.0 μg, about 0.03 μg to about 1.5 μg, about 0.05 μg to about 1.5 μg, about 0.1 μg to about 1.5 μg, about 0.5 μg to about 1.5 μg, about 1.0 μg to about 1.5 μg, about 0.03 μg to about 1.0 μg, about 0.05 μg to about 1.0 μg, about 0.1 μg to about 1.0 μg, about 0.5 μg to about 1.0 μg, about 0.03 μg to about 0.5 μg, about 0.05 μg to about 0.5 μg, about 0.1 μg to about 0.5 μg, about 0.03 μg to about 0.1 μg, about 0.05 μg to about 0.1 μg, or about 0.03 μg to about 0.05 μg.

According to some embodiments, the LNP/TNA is for administration at a dose of about 0.1 μg to about 1.0 μg, or about 0.1 μg to about 0.9 μg, about 0.1 μg to about 0.8 μg, about 0.1 μg to about 0.7 μg, about 0.1 μg to about 0.6 μg, about 0.1 μg to about 0.5 μg, about 0.1 μg to about 0.4 μg, about 0.1 μg to about 0.3 μg or about 0.1 μg to about 0.2 μg.

According to some embodiments, the LNP/TNA is for administration at a dose of about 0.1 μg to about 0.2 μg, for example a dose of about 0.1 μg, 0.11 μg, 0.12 μg, 0.13 μg, 0.14 μg, 0.15 μg, 0.16 μg, 0.17 μg, 0.18 μg, 0.19 μg or about 0.2 μg.

In one embodiment, repeat administrations of the therapeutic product can be made until the appropriate level of expression has been achieved. Thus, in one embodiment, a therapeutic nucleic acid can be administered and re-dosed multiple times. For example, the therapeutic nucleic acid can be administered on day 0. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the therapeutic nucleic acid.

In one embodiment, one or more additional compounds can also be included. Those compounds can be administered separately or the additional compounds can be included in the lipid particles (e.g., LNPs) of the disclosure. In other words, the lipid particles (e.g., LNPs) can contain other compounds in addition to the ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In one embodiment, the one or more additional compound can be a therapeutic agent. The therapeutic agent can be selected from any class suitable for the therapeutic objective. Accordingly, the therapeutic agent can be selected from any class suitable for the therapeutic objective. The therapeutic agent can be selected according to the treatment objective and biological action desired. For example, in one embodiment, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In one embodiment, if the LNP containing the ceDNA is useful for treating an infection, the additional compound can be an antimicrobial agent (e.g., an antibiotic or antiviral compound). In one embodiment, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In one embodiment, different cocktails of different lipid particles containing different compounds, such as a ceDNA encoding a different protein or a different compound, such as a therapeutic may be used in the compositions and methods of the disclosure. In one embodiment, the additional compound is an immune modulating agent. For example, the additional compound is an immunosuppressant. In some embodiments, the additional compound is immunostimulatory.

### REFERENCES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### EXAMPLES

The following examples are provided by way of illustration not limitation.

### Example 1: Delivery of ceDNA LNPs to the Retina

The mammalian retina is a thin layer of photosensitive tissue in the back of the eye. The retina is made up of various cell types, including photoreceptor (PR) cells and retinal pigmented epithelial (RPE) cells, a polarized monolayer of cells, whose apical side faces the neural retina. The RPE forms a part of the blood-retina barrier (BRB). The BRB is composed of both an inner and an outer barrier. The outer BRB refers to the barrier formed at the RPE cell layer and functions, in part, to regulate the movement of solutes and nutrients from the choroid to the sub-retinal space. In contrast, the inner BRB, similar to the blood brain barrier (BBB) is located in the inner retinal microvasculature and comprises the microvascular endothelium which line these vessels. Except for the vehicle, all LNP compositions were formulated with the eGFP mRNA cargo. At 24 h mice were imaged using fundus autofluorescence to visualize GFP expression. Mice were then sacrificed (at 24 h), eyes were enucleated, and processed for GFP ELISA or immunohistochemistry using anti-GFP.

It is to be understood that the term “GFP,” standalone or unless otherwise expressly indicated, as used in the Examples and accompanying figures, by default refer to enhanced green fluorescent protein. Furthermore, it is to be understood that the terms “OS” and “OD,” as used in the Examples and accompany figures, respectively refer to ocular sinister or left eye and ocular dexter or right eye.

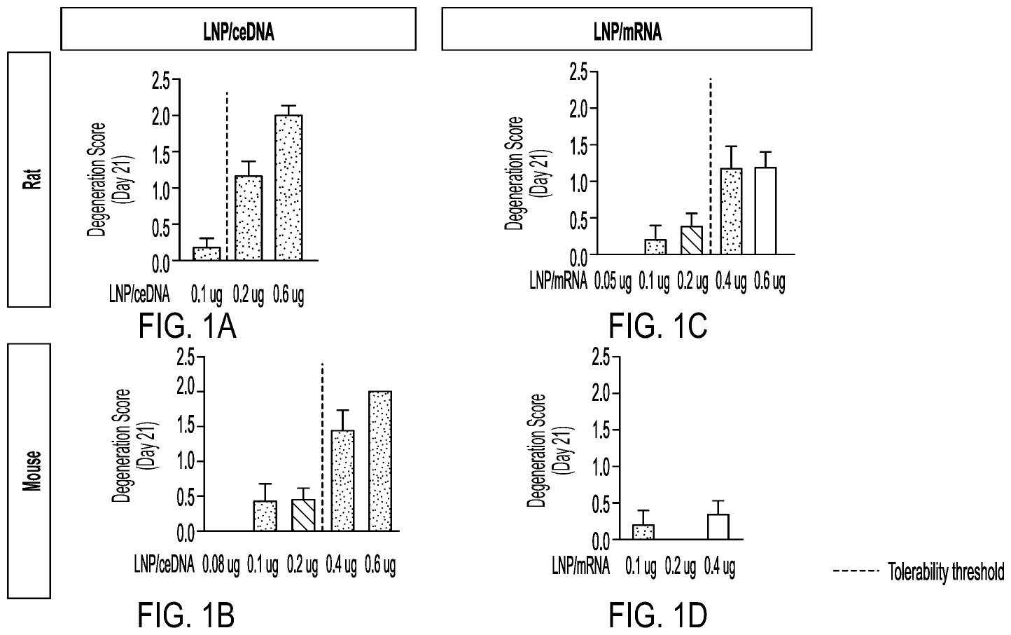

As an overview, FIGS. 1A-1D present graphs that show LNP-delivered mRNA and LNP-delivered ceDNA were tolerated in both mice and rats. LNP/ceDNA and LNP/mRNA compositions were administered at various doses. Degeneration scores (retinal degeneration) were determined at day 21.

### Example 2. Comparison of LNP-Delivered mRNA and AAV Expression Patterns in Mice Following Subretinal Injections

The objective of this study was to compare the expression of different test materials, including LNP-delivered GFP mRNA and gfp gene carried in an AAV vector, following subretinal injections in C57BL/6J mice. All animals were dosed subretinally (SR) with the test articles described in the Table 9A. Mice were pre-treated with a single dose of subcutaneous 0.5 mg/kg methylprednisolone. Furthermore, the GFP expression of transgenic mice was also compared to the GFP expression levels of the aforementioned test materials.

TABLE 9A
Experimental Design for C57BL/6J Mice
Mice: C57BL/6J

| | | Left eye | | | Right eye | | | | |
|---|---|---|---|---|---|---|---|---|---|
| | | | Dose | Volume | | Dose | Volume | | Euthanasia/ |
| Grp | | Treatment | (μg | (μl)/ | Treatment | (μg | (μl)/ | Study | Tissue |
| # | N | (Concentration) | or vg) | ROA | (Concentration) | or vg) | ROA | Endpoints | Collection |
| 1* | 8 | No Treatment | N/A | N/A | Vehicle | 0 | 1 μL | Full | Day 2: |
| | | | | | | | SR | Ocular | n = 2 eyes |
| | | | | | | | | Exams | for IHC, |
| | | | | | | | | (OEs): | n = 2 eyes |
| | | | | | | | | Baseline, | for |
| | | | | | | | | Days 7 | flatmount |
| | | | | | | | | and 21 | analysis |
| | | | | | | | | Color | and n = 6 |
| | | | | | | | | and | eyes for |
| 2* | 8 | Lipid A | 0.4 | 1 μL | Lipid A | 0.4 | | Cobalt | ELISA |
| | | LNP/GFP | | SR | LNP/GFP | | | blue | assay |
| | | mRNA | | | mRNA | | | fundus: | Day 28: |
| | | (0.4 μg/μl) | | | (0.4 μg/μl) | | | Days 1 | n = 6 eyes |
| | | | | | | | | and 27 | for |
| | | | | | | | | post- | flatmount |
| | | | | | | | | injection | analysis |
| | | | | | | | | OCT: | by |
| | | | | | | | | Baseline | Sponsor |
| | | | | | | | | (post- | |
| | | | | | | | | injection) | |
| 3* | 6 | AAV5-CAG- | 1E9 | | AAV5-CAG- | 1E9 | | and | Day 28: |
| | | GFP | | | GFP | | | Days 1 | n = 2 eyes |
| | | (1E9 vg/μL) | | | (1E9 vg/μL) | | | and 27 | for IHC, |
| | | | | | | | | post- | n = 2 eyes |
| | | | | | | | | injection | for |
| | | | | | | | | | flatmount |
| | | | | | | | | | analysis |
| | | | | | | | | | and n = 8 |
| | | | | | | | | | eyes |
| | | | | | | | | | frozen for |
| | | | | | | | | | ELISA |
| | | | | | | | | | assay |

Lipid A LNP/GFP mRNA is any lipid nanoparticle formulated using Lipid A as the ionizable or cationic lipid and the lipid nanoparticle contains mRNA that translates into GFP. The lipid nanoparticle is formulated using Lipid A in combination with any other lipid components (e.g., sterol, non-cationic lipid, PEGylated lipid) and at any molar ratios of Lipid A and the other lipid components).
AAV5-CAG-GFP is AAV serotype 5 virus (Capsid from AAV5 and ITR from AAV2) that expresses GFP under the control of CAG promoter.

Anima/Health and Acclimation: Animals were acclimated to the study environment for a minimum of 3 days prior to anesthesia. At the completion of the acclimation period, each animal was physically examined for determination of suitability for study participation. Examinations included the skin and external ears, eyes, abdomen, neurological, behavior, and general body condition. Animals determined to be in good health were released to the study.

Randomization and Study Identification: Animals were assigned to study groups according to Powered Research Standard Operating Procedures (SOPs). Animals were uniquely identified by corresponding cage card number.

Examination: Mortality and morbidity was done daily.

Body Weights: Baseline (pre-dose) and then at necropsy.

Surgicalprocedures-mice: On the day of the surgical procedure, mice were given buprenorphine 0.01-0.05 mg/kg subcutaneously. Animals were also given a cocktail oftropicamide (1.0%) and phenylephrine (2.5%) topically to dilate and proptose the eyes. Animals were then tranquilized for the surgical procedure with a ketamine/xylazine cocktail, and one drop of 0.5% proparacaine HCl was applied to both eyes. Eyes were prepared for aseptic surgical procedures. Alternatively, mice were tranquilized with inhaled isoflurane. The cornea was kept moistened using topical eyewash, and body temperature was maintained using hot pads as needed. A small pilot hole using the tip of a 30-gauge needle was made in the posterior sclera for subretinal injection using a 34-gauge needle and a 10 μl syringe. Following either injection procedure, 1 drop of Ofloxacin ophthalmic solution followed by eye lube was applied topically to the ocular surface and animals were allowed to recover from surgery. If at any time during the surgical procedure, the surgeon determines the injection was suboptimal, or not successful, the animal was euthanized and replaced. Mice were given atipamezole to reverse the xylazine effects (0.1-1.0 mg/kg).

Subretinal Injections: A 2-mm-long incision through the conjunctiva and Tenon's capsule was made to expose the sclera. A small pilot hole using the tip of a 30 gauge needle was made in the posterior sclera for subretinal injection using a 33 gauge needle and Hamilton syringe. Following either injection procedure, 1 drop of Ofloxacin ophthalmic solution followed by eye lube was applied topically to the ocular surface and animals were allowed to recover from surgery. If at any time during the surgical procedure, the surgeon determines the injection was suboptimal, or not successful, the animal was euthanized and replaced. Rats were given atipamezole to reverse the xylazine effects (0.1-1.0 mg/kg).

Ocular Examination: Ocular examination was done using a slit lamp biomicroscope to evaluate ocular surface morphology at the timepoints indicated in the table above. The scoring table below was used to assess anterior segment inflammation.

TABLE 9B
Inflammation Scoring Table
Clinical Grading of Anterior Segment Inflammation

| Grade^{a} | Criteria |
|---|---|
| 0 | No disease; eye is translucent and reflects light (red reflex) |
| 0.5 (trace) | Dilated blood vessels in the iris |
| 1 | Engorged blood vessels in the iris; abnormal pupil contraction |
| 2 | Hazy anterior chamber; decreased red reflux |
| 3 | Moderately opaque anterior chamber, but pupil still visible; |
| | dull red reflex |
| 4 | Opaque anterior chamber and obscured pupil; red reflex |
| | absent; proptosis |

^{a}Each higher grade includes the criteria of the preceding one.
Agarwal, R J, et al. Rodent Models of Experimental Autoimmune Uveitis. Methods in Molecular Medicine. 2004: Vol 102, pp 395-419.

Fundus Imaging: Both color and cobalt blue (eGFP expression) fundus imaging was done on all enrolled eyes at the timepoints in the experimental design table. Animals were given a cocktail of tropicamide (1.0%) and phenylephrine (2.5%) topically to dilate and proptose the eyes, and topical eye anesthetic was applied to the eyes (proparacaine 0.5% or similar). Color fundus photography was followed by cobalt blue photography.

Optical Coherence Tomography (OCT): On days as indicated by the experimental design table, all animals underwent OCT imaging procedures of the posterior section of the eye, to determine subretinal injection success and changes over time. Eyes were dilated using a cocktail of tropicamide HCL 1% and phenylephrine hydrochloride 2.5% for OCT 15 minutes prior to examination. Outer nuclear layer (ONL) thickness was measured at three positions (left, right, and center) from two OCT scans: one that goes through the injection site (bleb) and one that does not. All numerical thickness values were provided in a separate data report (spreadsheet), along with all associated/annotated OCT images.

Euthanasia Tissue Collections: At the timepoints indicated in the experimental design table, animals were humanely euthanized via carbon dioxide asphyxiation and death was confirmed by cervical dislocation or thoracotomy.

Flatmounts: Immediately after euthanasia, designated eyes were enucleated and fixed overnight at 4C in 4% PFA. Eyes were washed once in lx PBS, transferred into fresh PBS.

Immunohistochemistry: All eyes designated for IHC were enucleated, the approximate site of injection marked, then placed into lx PBS. A 2-3 mm deep incision was made at the limbus and eyes were fixed at room temperature in 4% paraformaldehyde in separately labeled vials for 1 hour. Eyes were then transferred into 0.1M phosphate buffer (PB), brought through a sequential sucrose gradient (10-30%, 1 hour each) followed by embedding in OCT medium and freezing on dry ice. Eyes were cryosectioned (141.1m sections) and stained with the following antibodies: 1/250 chicken anti-GFP, 1/100 rabbit anti-RPE65 (labeled as “RPE” or “RPE65” in the figures). and 1 250 mouse anti-Rhodopsin (labeled as “Rho” in the figures) followed by 1/200 anti-chicken Cy2, 1/200 anti-rabbit Cy3, 1/200 anti-mouse Cy5 and 1/1,000 DAPI (labeled as “DAPI” in the figures).

ELISA: Immediately after euthanasia, all eyes designated for ELISAs were enucleated and snap frozen in individual tubes and subsequently stored at 80° C. Results:

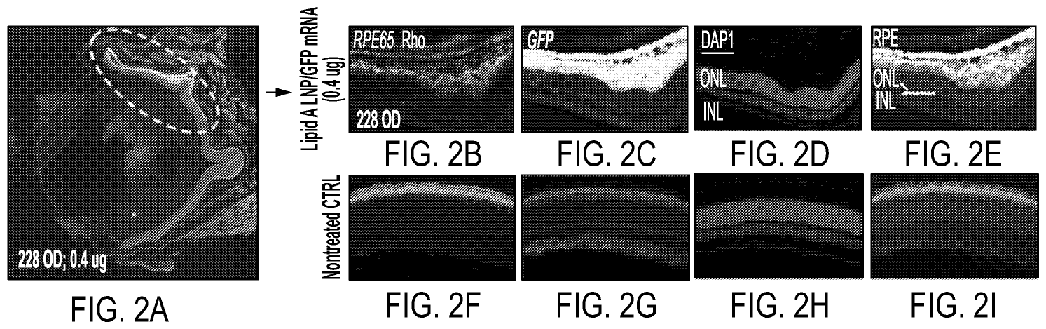

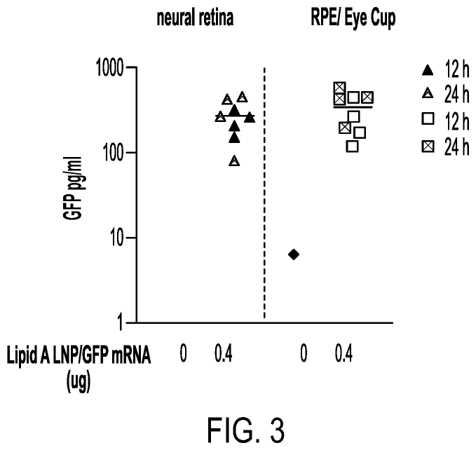

The fundus of the eye is the inside, back surface. It is made up of the retina, macula, optic disc, fovea and blood vessels. FIGS. 2A-2I show fundus imaging of Lipid A LNP/GFP mRNA treated mice (0.4 μg) (Group 2 as described in Table 9, FIGS. 2A-2E) compared to non-treated control mice (Group 1 as described in Table 9) (FIGS. 2F-2I). FIG. 3 is a graph that shows the amount of GFP in the neural retina and RPE/eye cup as determined by ELISA after dosing of wild type mice with Lipid A LNP/GFP mRNA treated mice (0.4 μg) (Group 2 as described in Table 9) at 12 hours and 24 hours post-treatment. Taken together, FIGS. 2A-2I and 3 demonstrate that Lipid A LNP/GFP mRNA transduces photoreceptors (PR) across the outer segment/inner segment (OS/IS) junction of the retina, as well as the retinal pigment epithelium (RPE) in the eye cup in mice.

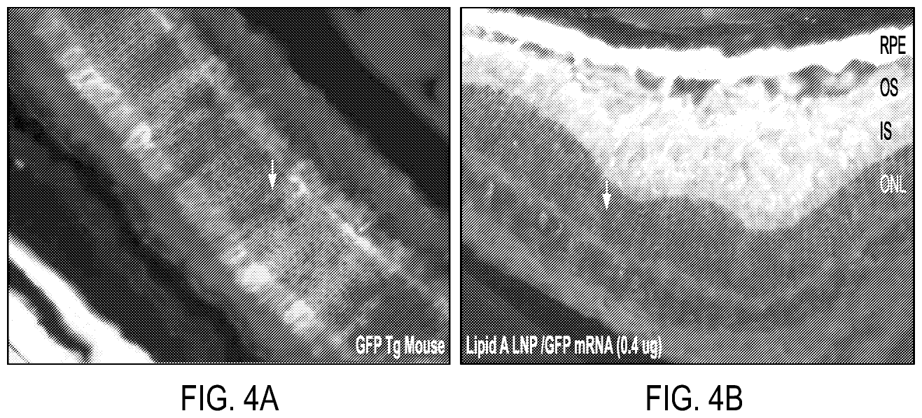

FIG. 4A and FIG. 4B show a comparison of GFP expression pattern in Lipid A LNP/GFP mRNA (0.4 μg) (FIG. 4B) compared to GFP transgenic mice (FIG. 4A). Transduction efficacy to the photoreceptors in the retina have been known to be challenging, even in GFP transgenic mice (FIG. 4A). As shown in FIG. 4B, robust GFP fluorescence could be seen in the outer and inner segments of the retina (OS/IS) as well as in the RPE in eye cup. Furthermore, a honeycomb GFP fluorescence pattern could be seen even past the inner segment (IS) of the retina in the outer nuclear layer (ONL) of the retina, which contains the nuclei of the rod photoreceptor cells, thus demonstrating successful transduction to the photoreceptors.

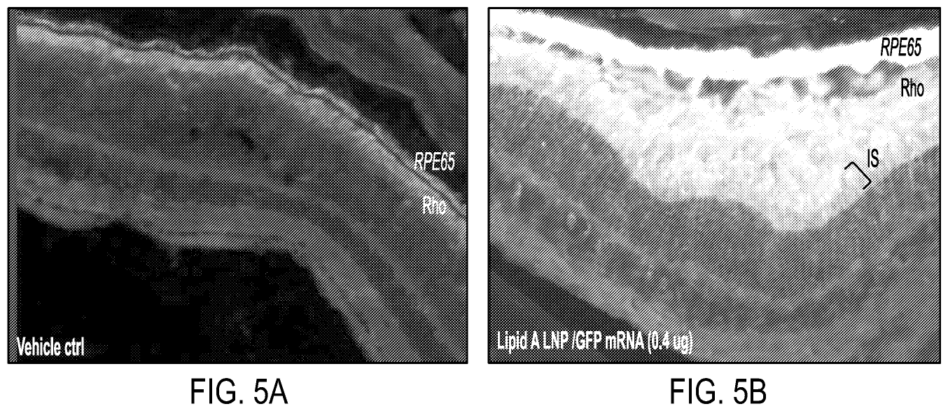

FIG. 5A and FIG. 5B show a comparison of GFP expression pattern in Lipid A LNP/GFP mRNA (0.4 μg) (FIG. 5B) compared to non-treated vehicle control mice (FIG. 5A). In both FIG. 5A and FIG. 5B, Rho was used as a marker of the photoreceptor (PR) outer segments. As can be seen in FIG. 5B, there was robust expression of GFP in the RPE and in the PR outer segments.

FIG. 6 is a graph that quantifies GFP expression. Importantly, FIG. 6 demonstrates that Lipid A LNP/GFP mRNA delivery in mice resulted in an even distribution of GFP expression within the neural retina and also eye cup.

FIGS. 7A-7E compare GFP expression in the neural retina and RPE in mice treated with Lipid A LNP/GFP mRNA (0.4 μg) and AAV5-CAG-GFP. As can be seen in the IHC images, AAV5-CAG-GFP-treated mice had fewer cells in the RPE with GFP expression at day 28 (FIGS. 7C, 7D), compared to Lipid A LNP/GFP mRNA treated mice at 24 hours (FIGS. 7A, 7B). As shown in the graph quantifying the results in FIG. 7E, mRNA expression from the Lipid A LNP/GFP mRNA construct at 24 hours matched levels of peak AAV5-CAG-GFP expression on Day 28, which were used as a benchmark.

The results presented above demonstrate that Lipid A LNP/GFP mRNA was successfully transduced into neural retina (photoreceptors) and RPE, and further, quantification of expression (FIG. 7E) showed that GFP expression levels of Lipid A LNP/GFP mRNA at 24 hours post-treatment were higherthan the GFP expression levels of AAV5-CAG-GFP at day 28 in both the neural retina and eye cup. Finally, the experiments shown above suggest that nucleic acid cargos (including DNA and mRNA) delivered to the retina via LNPs have an advantage over the AAV vector platform in terms of transduction because fewer cells in the RPE showed GFP expression in AAV-treated mice.

### Example 3. Evaluation of Expression of LNP-Delivered mRNA at Different Doses in Mice Following Subretinal Injection

The objective of this study was to evaluate GFP expression in wildtype mice following subretinal (SR)injections of different doses of LNP-delivered GFP mRNA and to identify a period of peak expression. 16 (+8 spares) male C57BL/6J mice were dosed subretinally (SR) with the test articles described below.

TABLE 10
Experimental Design for C57BL6J Mice

| | | | Left eye | | | Right eye | | | | Euthanasia/ |
|---|---|---|---|---|---|---|---|---|---|---|
| Grp | | | SR | Dose | Vol. | SR | Dose | Vol. | Study | Tissue |
| # | # | Strain | Treatment | (μg) | (μl) | Treatment | (μg) | (μl) | Endpoints | Collection |
| 1* | 4 | C57B1/6J | No | N/A | N/A | Vehicle | 0 | 1 | Full | n = 4 eyes |
| | | | Treatment | | | | | | Ocular | will be |
| | | | | | | | | | Exams | enucleated |
| | | | | | | | | | (OEs): | and snap |
| 2* | 4 | | Lipid A | 0.2 | 1 | Lipid A | 0.2 | 1 | Baseline | frozen at |
| | | | LNP/GFP | | | LNP/GFP | | | OCT: | 12 and 24 |
| | | | mRNA | | | mRNA | | | Baseline | hours |
| | | | (0.2 μg/μL) | | | (0.2 μg/μL) | | | (post- | post- |
| | | | | | | | | | injection), | injection |
| 3* | 4 | | Lipid A | 0.4 | 1 | Lipid A | 0.4 | 1 | and 24 | |
| | | | LNP/GFP | | | LNP/GFP | | | hours | |
| | | | mRNA | | | mRNA | | | post- | |
| | | | (0.4 μg/μL) | | | (0.4 μg/μL) | | | injection | |
| | | | | | | | | | Color and | |
| 4* | 4 | | Lipid A | 1 | 1 | Lipid A | 1 | 1 | Cobalt | |
| | | | LNP/GFP | | | LNP/GFP | | | Blue | |
| | | | mRNA | | | mRNA | | | Fundus: | |
| | | | (1 μg/μL) | | | (1 μg/μL) | | | 12 hours | |
| | | | | | | | | | (n = 4 | |
| | | | | | | | | | eyes/group) | |
| | | | | | | | | | and 24 | |
| | | | | | | | | | hours | |
| | | | | | | | | | post- | |
| | | | | | | | | | injection | |

Lipid A LNP/GFP mRNA is any lipid nanoparticle formulated using Lipid A as the ionizable or cationic lipid and the lipid nanoparticle contains mRNA that translates into GFP. The lipid nanoparticle is formulated using Lipid A in combination with any other lipid components (e.g., sterol, non-cationic lipid, PEGylated lipid) and at any molar ratios of Lipid A and the other lipid components.

Procedures related to animal health examination and acclimation, randomization and study identification, mortality and morbidity examination, body weight examination, surgery, ocular examination, optical coherence tomography, fundus imaging, and euthanasia/tissue collection for this Example 3 study are as described in Example 2 with minor modifications.

### Results:

FIGS. 8A and 8B are graphs that quantitate GFP expression by ELISA in the neural retina (with photoreceptors or PR) and eyecup (with retinal pigment epithelium or RPE cells) at increasing doses (0.2 μg, 0.4 μg, 1.0 μg) at 12 and 24 hours, with the GFP concentration expressed as ng/eye (FIG. 8A) and ng/μg cargo (FIG. 8B). Both sets of graphs indicate that GFP expression could be detected at 12 hours post-treatment and the expression levels further increased at 24 hours post-treatment, in both neural retina and eyecup. Significantly, both neural retina and eyecup were saturated with GFP at the lowest dose of 0.2 μg.

### Example 4. Comparison of Lipid-Delivered mRNA and AAV Vector Expression Patterns in Mice and Rats Following Subretinal Injections

The objective of this study was to compare the expression of different test doses of LNP-delivered mRNA following subretinal injections in Sprague Dawley rats and C57BL/6J mice (dose-matched). This study was designed to further develop a delivery platform to treat humans with degenerative retinal disease. 20 (+8 spares) male C57BL/6J mice and 20 (+8 spares) male Sprague Dawley rats were dosed subretinally (SR) with the test articles described below. Pursuant to the results observed in Example 2 where a 0.2 μg dose was demonstrated to be sufficient to saturate GFP expression in both neural retina and eye cup, the mice in this study were administered at lower doses such as 0.03 μg and 0.1 μg as compared to the 0.4 μg dose administered in Example 2 described above. As for the rats, Lipid A LNP/GFP mRNA was dose matched at higher doses such as 0.3 μg and 1.2 μg (i.e., higher than 0.1 μg that was dosed in Example 2 study). Mice on study (Group 1-4) were pre-treated with a single dose of subcutaneous 0.5 mg/kg methylprednisolone. Rats on study (Groups 5-8) were treated with a daily treatment of 0.5 mg/kg methylprednisolone (subcutaneously, SC) beginning on Day-1 and concluding on Day 28.

TABLE 11A
Experimental Design for C57BL/6J Mice
Mice: C57BI/6J

| | | Left eye | | | Right eye | | | | |
|---|---|---|---|---|---|---|---|---|---|
| | | | Dose | Volume | | Dose | Volume | | Euthanasia/ |
| Grp | | Treatment | (μg | (μl)/ | Treatment | (μg | (μl)/ | Study | Tissue |
| # | N | (Concentration) | or vg) | ROA | (Concentration) | or vg) | ROA | Endpoints | Collection |
| 1* | 5 | No Treatment | N/A | N/A | Vehicle | 0 | 1 μL | Full | Day 1: |
| | | | | | | | SR | Ocular | IHC: n = 2 |
| | | | | | | | | Exams | eyes/group |
| | | | | | | | | (OEs): | Sponsor- |
| | | | | | | | | Baseline | developed |
| 2* | 5 | Lipid A/GFP | 0.03 | 1 μL | Lipid A/GFP | 0.03 | | Color | assay: n = 4 |
| | | mRNA | | SR | mRNA | | | and | eyes/group |
| | | (0.03 ug/μL) | | | (0.03 ug/μL) | | | Cobalt | Day 28: |
| | | | | | | | | blue | IHC/TUN |
| | | | | | | | | fundus: | EL: n = 4 |
| | | | | | | | | Day 1 | eyes/group |
| 3* | 5 | Lipid A/GFP | 0.1 | | Lipid A/GFP | 0.1 | | OCT: | |
| | | mRNA | | | mRNA | | | Baseline | |
| | | (0.1 ug/μL) | | | (0.1 ug/μL) | | | (post- | |
| | | | | | | | | injection) | |
| | | | | | | | | 24 | |
| 4* | 5 | Lipid A/GFP | 0.4 | | Lipid A/GFP | 0.4 | | hours, | |
| | | mRNA | | | mRNA | | | (n = 3 | |
| | | (0.4 ug/μL) | | | (0.4 ug/μL) | | | terminal) and | |
| | | | | | | | | Day 21 | |
| | | | | | | | | (remaining | |
| | | | | | | | | n = 2) | |
| | | | | | | | | post- | |
| | | | | | | | | injection | |

Lipid A LNP/GFP mRNA is any lipid nanoparticle formulated using Lipid A as the ionizable or cationic lipid and the lipid nanoparticle contains mRNA that translates into GFP. The lipid nanoparticle is formulated using Lipid A in combination with any other lipid components (e.g., sterol, non-cationic lipid, PEGylated lipid) and at any molar ratios of Lipid A and the other lipid components.

TABLE 11B
Experimental Design for Sprague-Dawley Rats
Rats: Sprague-Dawley

| | | Left eye | | | Right eye | | | | |
|---|---|---|---|---|---|---|---|---|---|
| | | | Dose | Volume | | Dose | Volume | | Euthanasia/ |
| Grp | | Treatment | (μg | (μl)/ | Treatment | (μg | (μl)/ | Study | Tissue |
| # | N | (Concentration) | or vg) | ROA | (Concentration) | or vg) | ROA | Endpoints | Collection |
| 5^{§} | 5 | No Treatment | N/A | N/A | Vehicle | 0 | 2.5 μL | Full | Day 1: |
| | | | | | | | SR | Ocular | IHC: n = 2 |
| | | | | | | | | Exams | eyes/group |
| | | | | | | | | (OEs): | ELISA |
| | | | | | | | | Baseline | assay: n = 4 |
| 6^{§} | 5 | Lipid A | 0.1 | 2.5 μL | Lipid A | 0.1 | | Color | eyes/group |
| | | LNP/GFP | | SR | LNP/GFP | | | and | Day 28: |
| | | mRNA | | | mRNA | | | Cobalt | IHC/TUN |
| | | (0.04 μg/μL) | | | (0.04 μg/μL) | | | blue | EL: n = 4 |
| | | | | | | | | fundus: | eyes/group |
| | | | | | | | | Day 1 | |
| 7^{§} | 5 | Lipid A | 0.3 | | Lipid A | 0.3 | | OCT: | |
| | | LNP/GFP | | | LNP/GFP | | | Baseline | |
| | | mRNA | | | mRNA | | | (post- | |
| | | (0.12 μg/μL) | | | (0.12 μg/μL) | | | injection) | |
| | | | | | | | | 24 hours, | |
| 8^{§} | 5 | Lipid A | 1.2 | | Lipid A | 1.2 | | (n = 3 | |
| | | LNP/GFP | | | LNP/GFP | | | terminal) and | |
| | | mRNA | | | mRNA | | | Day 21 | |
| | | (0.48 μg/μL) | | | (0.48 μg/μL) | | | (remaining | |
| | | | | | | | | n = 2) | |
| | | | | | | | | post- | |
| | | | | | | | | injection | |

Lipid A LNP/GFP mRNA is any lipid nanoparticle formulated using Lipid A as the ionizable or cationic lipid and the lipid nanoparticle contains mRNA that translates into GFP. The lipid nanoparticle is formulated using Lipid A in combination with any other lipid components (e.g., sterol, non-cationic lipid, PEGylated lipid) and at any molar ratios of Lipid A and the other lipid components.

Procedures related to animal health examination and acclimation, randomization and study identification, mortality and morbidity examination, body weight examination, surgery, ocular examination, optical coherence tomography, fundus imaging, euthanasia/tissue collection, IHC, and ELISA for this Example 4 study are as described in Example 2 with minor modifications.

ApopTag Red TUNEL (Powered Research; Day 28 eyes only): All eyes designated for TUNEL were enucleated, the approximate site of injection marked with a fluorescent tissue marker and then placed into lx PBS. A 2-3 mm deep incision was made at the limbus and eyes were fixed at room temperature in 4% paraformaldehyde in separately labeled vials for 1 hour. Eyes were then transferred into 0.1M phosphate buffer (PB), brought through a sequential sucrose gradient (10-30%, 1 hour each) followed by embedding in OCT medium and freezing on dry ice. Eyes were cryosectioned (14 μm sections). Two (2) slides per eye (32 eyes/64 slides) within the central area of the fluorescent tissue marker (marks approximate site of injection) were selected for TUNEL. Slides were post-fixed in 1% PFA in PBS for 10 minutes at room temperature then washed 2×5 minutes in PBS. Seventy-five (75) microliters of equilibration buffer was applied for 10 minutes at RT; flexible plastic coverslips from the kit can be used to conserve volume of reagents needed. Excess liquid was removed and working strength TdT enzyme was applied for 60 minutes at 37° C. in a humidified chamber. Working strength STOP/WASH buffer was applied for 10 minutes. Slides were washed 3 x 1 minute with PBS and Cy3-conjugated anti-digoxin antibody (Jackson Immuno) was applied for 30 minutes in a dark, humidified chamber; alternatively, slides can be placed in Sequenza slide rack. Wash 3×10 minutes in PBS. TUNEL labeled cells will appear red. Slides were then counter-stained with the following antibodies: 1/250 rabbit anti-RPE65 and 1/1,000 DAPI as described in the immunofluorescence section above.

### Results:

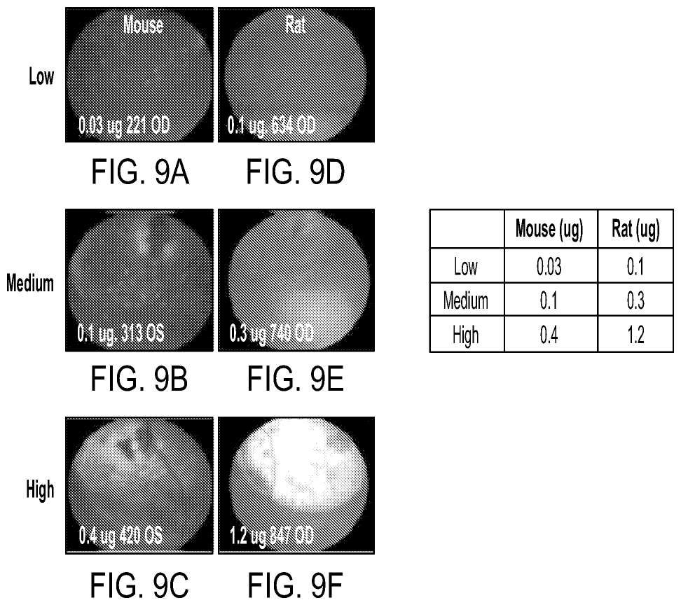

When LNP-delivered mRNA such as Lipid A LNP/GFP mRNA was dose matched in the mouse and rat models, GFP expression by fundus in the rat was found to be comparable to that in the mouse. As shown in FIGS. 9E and 9F, Lipid A LNP/GFP mRNA given at the medium and high doses (0.3 μg and 1.2 μg, respectively) achieved expression levels in rats that were comparable to the expression levels of Lipid A LNP/GFP mRNA given at the medium and high doses mice (0.1 μg and 0.4 μg, respectively, see FIGS. 9B and 9C)

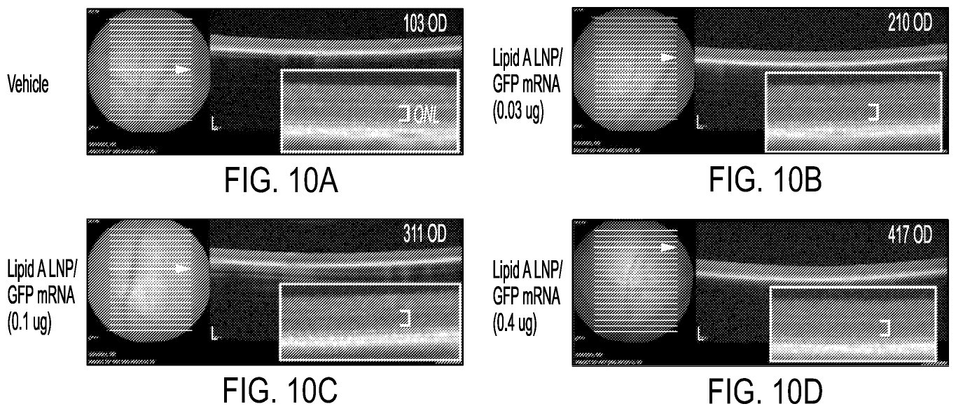

In addition to the demonstrated saturation of GFP expression in both neural retina and eyecup of mice treated with a low dose of 0.2 μg Lipid A LNP/GFP mRNA (see Example 2 above, in particular FIGS. 8A and 8B), the images in FIGS. 10B-10D show that no retinal degeneration occurred a day 1 after the mice were administered with increases Lipid A LNP/GFP mRNA doses of 0.03 μg, 0.1 μg, and 0.4 μg (see vehicle in FIG. 10A for reference), thereby indicating a large tolerability window for LNP-delivered mRNA such as Lipid A LNP/GFP mRNA.

### Example 5. Evaluation of Safety and Test Article Expression of a Therapeutic Following Subretinal Injection in Wildtype Mice

The objective of this study was to compare the expression of different LNP compositions formulated with different ionizable lipids (each carrying GFP/mRNA cargo) following subretinal injections into wildtype C57BL/6J mice. This study was designed to further develop a delivery platform to treat humans with degenerative retinal disease. 36 (+12 spares) male C57BL/6J mice were dosed subretinally (SR) with the test articles described in the Experimental Design below. Mice on study were pre-treated with a single dose of subcutaneous 0.5 mg/kg methylprednisolone.

TABLE 12
Experimental Design for C57BL/6J Mice
Mice: C57BI/6J

| | | OS | | | OD | | | | |
|---|---|---|---|---|---|---|---|---|---|
| | | Tx | Dose | Volume | Tx | Dose | Volume | | Euthanasia/ |
| Grp | | [Final | (μg | (μl)/ | [Final | (μg | (μl)/ | Study | Tissue |
| # | N | Concentration] | or vg) | ROA | Concentration] | or vg) | ROA | Endpoints | Collection |
| 1* | 6 | No Treatment | N/A | N/A | Vehicle | 0 | 1 μL | Full | Day 2^{∇}: |
| | | | | | | | SR | Ocular | n = 2 |
| | | | | | | | | Exams | eyes/group |
| | | | | | | | | (OEs): | fixed for |
| | | | | | | | | Baseline, | GFP IHC |
| 2* | 6 | Lipid A | 0.2 | 1 μL | Lipid A | 0.2 | | Days 7 | and n = 6 |
| | | LNP/GFP | | SR | LNP/GFP | | | and 21 | eyes/group |
| | | mRNA | | | mRNA | | | Color | snap |
| | | (0.2 μg/μL) | | | (0.2 μg/μL) | | | and | frozen for |
| | | | | | | | | Cobalt | ELISA |
| 3* | 6 | MC3 | 0.2 | | MC3 | 0.2 | | blue | assay |
| | | LNP/GFP | | | LNP/GFP | | | fundus: | Day 29: |
| | | mRNA | | | mRNA | | | 24 | n = 4 |
| | | (0.2 μg/μL) | | | (0.2 μg/μL) | | | hours | eyes/group |
| | | | | | | | | post- | snap |
| 4* | 6 | CTRL Lipid | 0.2 | | CTRL Lipid | 0.2 | | injection | frozen for |
| | | Z LNP 1/GFP | | | Z LNP 1/GFP | | | OCT: | ELISA |
| | | mRNA | | | mRNA | | | Baseline | assay |
| | | (0.2 μg/μL) | | | (0.2 μg/μL) | | | (post- | |
| | | | | | | | | Injection), | |
| 5* | 6 | CTRL Lipid | 0.2 | | CTRL Lipid | 0.2 | | 24 | |
| | | Z LNP 2/GFP | | | Z LNP 2/GFP | | | hours | |
| | | mRNA | | | mRNA | | | and | |
| | | (0.2 μg/μL) | | | (0.2 ug/μL) | | | Days 14, | |
| 6* | 6 | Lipid 58 | 0.2 | | Lipid 58 | 0.2 | | and 28 | |
| | | LNP/GFP | | | LNP/GFP | | | post- | |
| | | mRNA | | | mRNA | | | injection | |
| | | (0.2 μg/μL) | | | (0.2 μg/μL) | | | | |

Lipid A LNP/GFP mRNA is any lipid nanoparticle formulated using Lipid A as the ionizable or cationic lipid and the lipid nanoparticle contains mRNA that translates into GFP. The lipid nanoparticle is formulated using Lipid A in combination with any other lipid components (e.g., sterol, non-cationic lipid, PEGylated lipid) and at any molar ratios of Lipid A and the other lipid components.
CTRL Lipid Z LNP 1/GFP mRNA and CTRL Lipid Z LNP 2/GFP mRNA are each a lipid nanoparticle composition formulated using CTRL Lipid Z, which is a different class of ionizable lipid that lacks the disulfide bond in the headgroup that is present in Lipid A. CTRL Lipid Z LNP 1/GFP mRNA and CTRL Lipid Z LNP 2/GFP mRNA were prepared using different processes and also vary in the other lipid components such as the PEGylated lipid, sterol, and/or non-cationic lipid. (CTRL = control).

Procedures related to animal health examination and acclimation, randomization and study identification, mortality and morbidity examination, body weight examination, surgery, ocular examination, optical coherence tomography, fundus imaging, euthanasia/tissue collection, IHC, and ELISA for this Example 5 study are as described in Example 2 with minor modifications. Results:

FIGS. 11A-11E show color fundus imaging of mice eyes at day 2 post-treatment via subretinal injections with various LNP compositions formulated with GFP mRNA and with different ionizable lipids as described in Table 12 (all 0.2 μg dose) while FIGS. 11F-11J show corresponding cobalt blue fundus imaging (for GFP expression) of the same mice eye samples. Specifically, FIGS. 11A and 11F are images of mice eyes treated with 0.2 μg of Lipid A LNP/GFP mRNA; FIGS. 11A and 11G are images of mice eyes treated with 0.2 μg of Lipid A LNP/GFP mRNA; FIGS. 11B and 11F are images of mice eyes treated with 0.2 μg of MC3 LNP/GFP mRNA; FIGS. 11C and 11G are images of mice eyes treated with 0.2 μg of CTRL Lipid Z LNP 1/GFP mRNA; FIGS. 11D and 11H are images of mice eyes treated with 0.2 μg of CTRL Lipid Z LNP 2/GFP mRNA; and FIGS. 11E and 11J are images of mice eyes treated with 0.2 μg of Lipid 58 LNP/GFP mRNA.

The color fundus images of FIGS. 11B-11D indicate that LNP compositions formulated with MC3 or CTRL Lipid Z had severe toxicity that led to choroidal ischemia, which is interruption of vascular or circulatory flow that would result in blindness. The predominantly white and translucent areas seen in FIGS. 11B-11D are indicative of dead retinal cells. The corresponding cobalt blue fundus images of FIGS. 11G-11I, not surprisingly, showed minimal GFP expression.

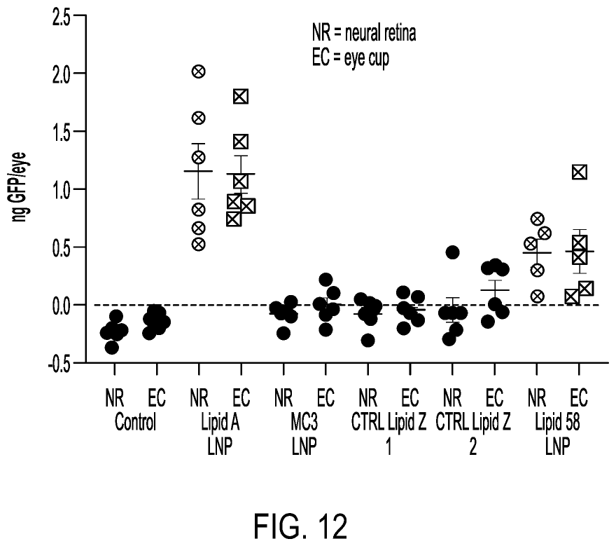

In contrast, the color fundus images of FIGS. 11A and 11E show that the retinal cells of mice eyes treated with LNP compositions formulated with Lipid A or Lipid 58 maintained a healthy, pink reddish color, thereby indicating that these compositions were well-tolerated and did not lead to choroidal ischemia; and good GFP expression was also observed in FIGS. 11F and 11J. FIG. 12 is a graph that quantifies GFP expression in both the neural retina and eye cup. The quantification data is in agreement with the observations noted in FIGS. 11F-11J in that LNP compositions formulated with Lipid A or Lipid 58 had good GFP expression, while LNP compositions formulated with MC3 or CTRL Lipid Z.

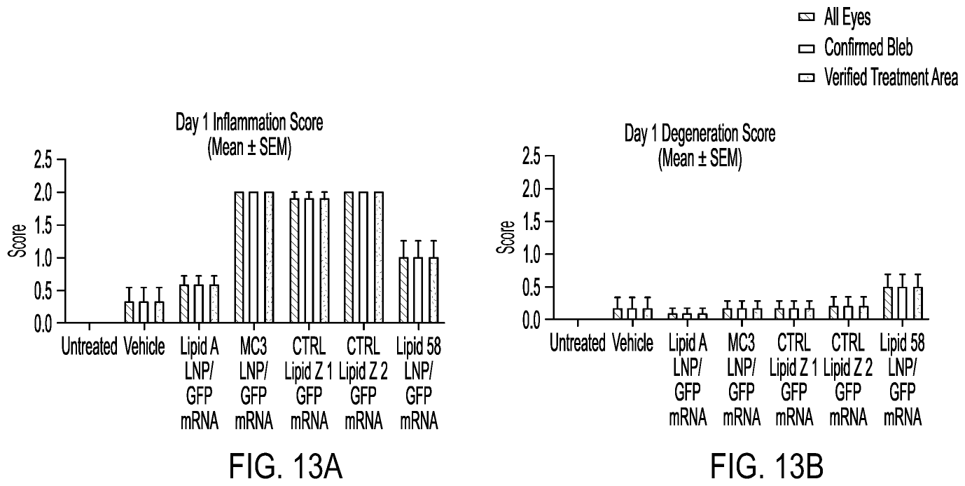

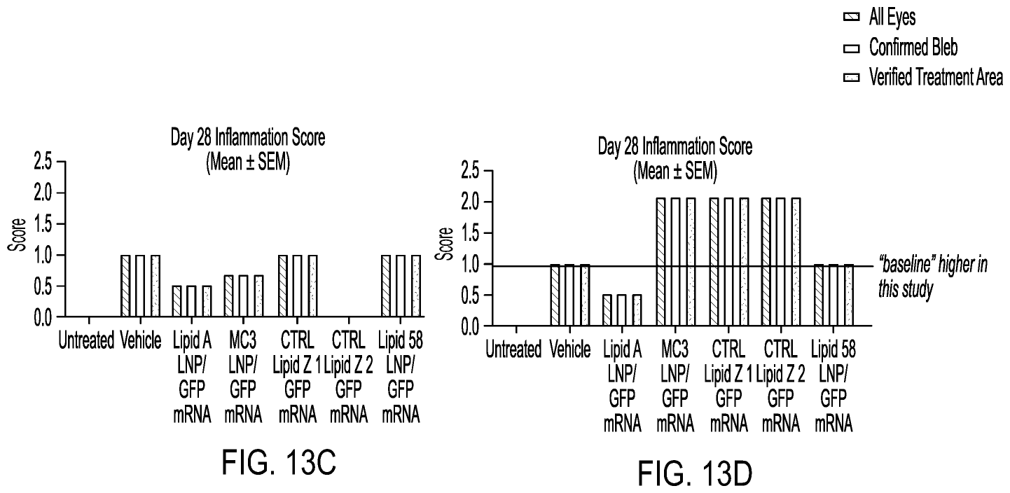

FIGS. 13A-13B are graphs respectively showing day 1 inflammation scores and degeneration scores of mice eyes post-treatment via subretinal injections with various LNP compositions formulated with GFP mRNA and with different ionizable lipids as described in this example (all 0.2 μg dose); whereas FIGS. 13C-13D are graphs respectively showing day 1 inflammation scores and degeneration scores of the same samples. Consistent with the toxicity observed in the fundus imaging described above, LNP compositions formulated with either MC3 or CTRL Lipid Z recorded inflammation scores as high as-2.0 at day 1; while in contrast, Lipid A LNP/GFP mRNA and Lipid 58 LNP/GFP mRNA respectively recorded inflammation scores of <0.5 and <1.0.

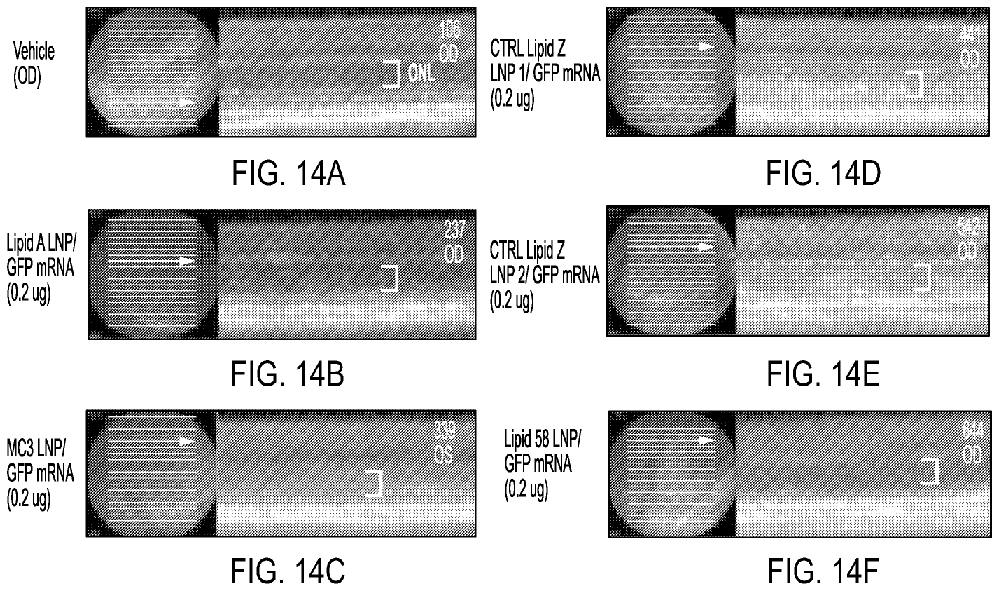

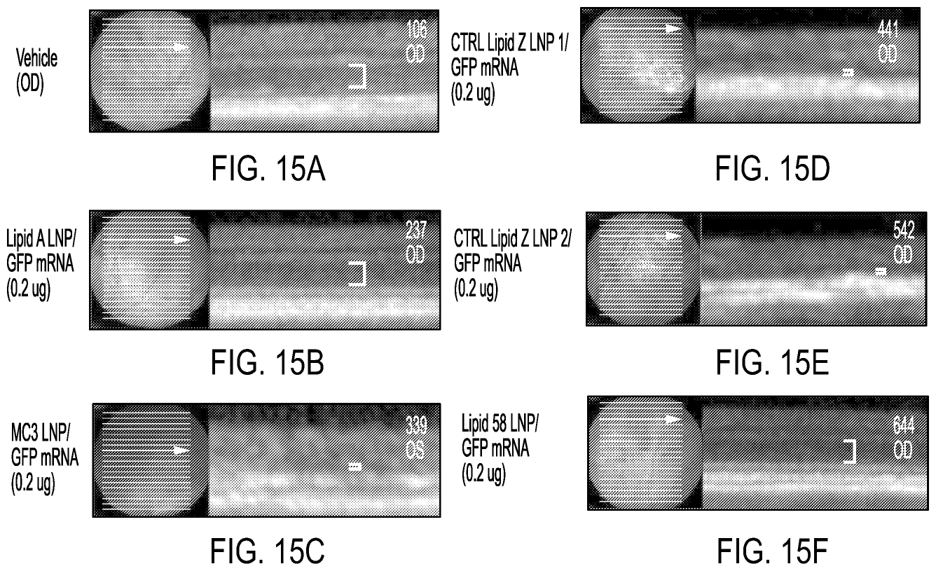

Moreover, at day 28, degeneration scores as high as ˜2.0 were observed in LNP compositions formulated with either MC3 or CTRL Lipid Z; and such high degeneration scores were corroborated by the thinning of the ONL layer or retinal degeneration seen in OCT images taken on day 28 (see FIGS. 15C-15E and using FIG. 15A vehicle as a reference). In contrast, at day 28, Lipid A LNP/GFP mRNA and Lipid 58 LNP/GFP mRNA respectively recorded degeneration scores of <0.5 and ˜1.0 and their corresponding OCT images in FIG. 15B and FIG. 15F (using FIG. 15A vehicle as a reference) corroboratively indicate that the ONL layer maintained a healthy thickness. At day 1, none of the samples exhibited any retinal degradation (see FIGS. 14A-14F, using FIG. 14A vehicle as a reference.

### Example 6. 4-Week Tolerability and Expression Study following Subretinal Administration in Female Cynomolgus Monkeys

The objective of this study was to evaluate the tolerability and expression of LNP-delivered nucleic acid cargo, such as LNP-delivered mRNA, when administered as a single dose via subretinal injection to female cynomolgus monkeys, utilizing a Lipid A LNP/GFP mRNA at either a low dose of 0.6 μg or a high dose of 3.0 μg. Animals were also administered an immunosuppressant once daily through Dosing Phase 1 and continuing through Day 1 (interim sacrifice animals in Groups 2 and 3), 7 (Group 4), or 28 (terminal sacrifice animals in Groups 1, 2, and 3) of Dosing Phase 2. Interim sacrifice animals in Groups 2 and 3 were sacrificed on Day 2 at 24 hours (+2 hours) postdose. After dosing, terminal sacrifice animals in Groups 1, 2, and 3 were observed postdose for 28 days (Day 29 terminal sacrifice) to assess the reversibility or persistence of any effects.

The subretinal route of administration was chosen because it is the intended human therapeutic route. The high dose was intended to be the maximum feasible dose based on subretinal bleb administration limits in this species (150 iL/bleb). The low dose was intended to understand the dose-responsiveness of the test articles.

The cynomolgus monkey was selected as the relevant species because of the similarity of monkeys to humans with respect to the anatomy and physiology of the retina.

TABLE 13A
Dosing Phase 1 (Immunosuppressant)

| | | | Dose | | Dose | Number |
|---|---|---|---|---|---|---|
| | Dose | Days of | Volume | Dose Level | Concentration | of |
| Group^{a} | Regimen | Dosing | (mL/kg) | (mg/kg/day) | (mg/mL) | Females |
| 1 (Control) | Prednisolone | 1 through 14 | 0.33 | 1 | 3 | 1 |
| 2 (Low) | Prednisolone | 1 through 14 | 0.33 | 1 | 3 | 2 |
| 3 (High) | Prednisolone | 1 through 14 | 0.33 | 1 | 3 | 2 |
| 4 (Extras) | Prednisolone | 1 through 14 | 0.33 | 1 | 3 | 1 |

TABLE 13B
Dosing Phase 2 (Immunosuppressant)

| | | | Dose | | Dose | Number |
|---|---|---|---|---|---|---|
| | Dose | | Volume | Dose Level | Concentration | of |
| Group^{a} | Regimen | Days of Dosing | (mL/kg) | (mg/kg/day) | (mg/mL) | Females |
| 1 (Control) | Prednisolone | 1 through 28 | 0.33 | 1 | 3 | 1 |
| 2 (Low) | Prednisolone | 1 or 1 through 28 | 0.33 | 1 | 3 | 2 |
| 3 (High) | Prednisolone | 1 or 1 through 28 | 0.33 | 1 | 3 | 2 |
| 4 (Extras) | Prednisolone | 1 through 7; 9, 11, | 0.33 | 1 | 3 | 1 |
| | | 13, and 15 | 0.167 | 0.5 | 3 | |
| | | 17, 19, 21 | | | | |

TABLE 13C
Dosing Phase 3 (Vehicle Control and Test Article)

| | | | | | | | | Dose | | |
|---|---|---|---|---|---|---|---|---|---|---|
| | | | | Dose Volume | | Dose Level^{c} | | Concentration | | |
| | Dose Regimen | | Days | (μL/eye) | | (μg/eye) | | (mg/mL) | | Number |
| | Left | Right | of | Left | Right | Left | Right | Left | Right | of |
| Group^{b} | Eye | Eye | Dosing | Eye | Eye | Eye | Eye | Eye | Eye | Females |
| 1 | Not | Vehicle | 1 | NA | 150 | NA | 0 | NA | 0 | 1 |
| (Control)^{d} | Dosed | | | | | | | | | |
| 2 (Low) | Test Article | Test Article | 1 | 150 | 150 | 6 | 6 | 0.04 | 0.04 | 2 |
| 3 (High) | Test Article | Test Article | 1 | 150 | 150 | 30 | 30 | 0.2 | 0.2 | 2 |
| 4 (Extras) | Not Dosed | Not Dosed | NA | NA | NA | NA | NA | NA | NA | 1 |

NA = Not Applicable; TA = Test Article.

Animals in Groups 1, 2, and 3 and designated for the terminal sacrifice were administered.

Fundus Autofluorescence Imaging: Animals were fasted (for at least 10 hours) before the procedure. Animals were be anesthetized with ketamine and maintained on sevoflurane. The anesthesia regimen may be adjusted as necessary, at the discretion of the attending Veterinarian and/or Study Director. Pupils were dilated with a mydriatic agent. Images were taken with a Phoenix Micron X® instrument. Fundus autofluorescence images included posterior pole, the bleb (as much as possible), and any areas of hyperfluorescence (if possible).

Fundus Ocular Photography: Animals were fasted (for at least 10 hours) before the procedure. Animals were anesthetized with ketamine and dexmedetomidine (for dosing). The anesthesia regimen was adjusted as necessary. Photographs of both eyes were taken with a digital fundus camera. Color photographs were used to document the location and appearance of the subretinal bleb(s) following dosing. For the left eye of Group 1, representative control images were taken. These images may be used as a reference to identify the treated area at subsequent OCT intervals.

Optical Coherence Tomography (OCT): Animals were fasted (for at least 10 hours) before the procedure. Animals were anesthetized with ketamine and maintained on sevoflurane. The anesthesia regimen was adjusted as necessary, at the discretion of the attending Veterinarian and/or Study Director. Pupils were dilated with a mydriatic agent.

Spectral domain optical coherence tomography (OCT or sdOCT) and imaging were done in a manner to obtain axial views of the retinal surface in the posterior fundus. The instruments were be set to perform standard retinal scans (macular volume scans and/or line scans and/or circle scans).

During the dosing phase, attempts were made to collect scans within the treated area (subretinal bleb) as well as an untreated region (outside the subretinal bleb) at approximately the same distance from the optic nerve head (ONH). Feasibility of these scans will depend on the size, location, and visibility of the subretinal bleb.

As needed, color images collected post-dose on Day 2 were used as a reference to identify the treated area at dosing phase OCT intervals.

Histology: Both eyes from all terminal sacrifice animals, unless noted as missing, were trimmed in a vertical plane by taking a cross section down either side of the optic nerve, perpendicular to the posterior ciliary arteries, and embedded in paraffin (i.e., a central portion). In addition, the remaining temporal calotte were embedded in paraffin.

Embedded tissues were sectioned at a nominal 5 μm and stained with hematoxylin and eosin (H & E). From each eye, the central portion was sectioned to facilitate examination of the optic disc and optic nerve. The temporal calotte was sectioned through the fovea to facilitate examination of the subretinal bleb, as applicable. Results:

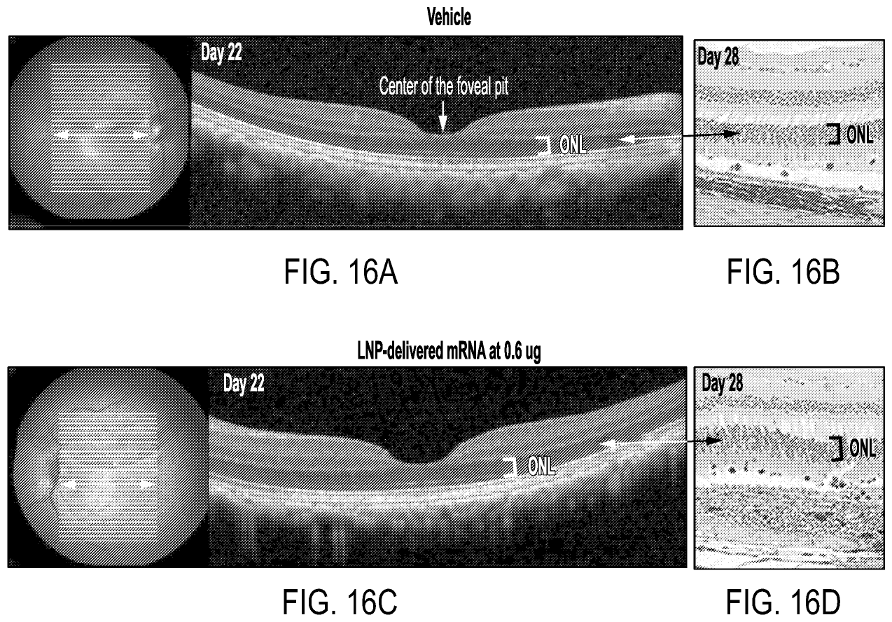

FIGS. 16A-16D show the images of OCT (taken at day 22) and H&E qualitative analysis (taken at day 28) for the vehicle control and the low dose of 6 μg. At the 6 μg dose, Lipid A LNP/GFP mRNA appeared to be well-tolerated in monkeys because there was no meaningful ONL thinning or retinal degradation being observed.

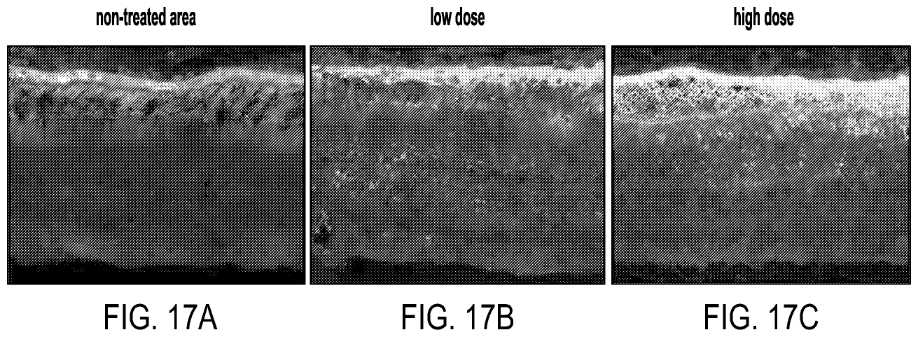

FIGS. 17A-17C are IHC images taken of the untreated area that serves as negative control (FIG. 17A), 6 μg low dose treatment (FIG. 17B), and 30 μg high dose (FIG. 17C), 24 hours post-treatment. GFP expression was seen in both low and high doses, with the expression levels being higher or more robust at the high dose, thereby indicating dose responsiveness in monkeys.

Taken together, the results obtained in this Example indicate that LNP-delivered nucleic acid cargo, such as Lipid A LNP-delivered mRNA, is well-tolerated and achieves good expression in monkeys.

What is claimed is:

1. A pharmaceutical composition comprising a lipid nanoparticle (LNP), a therapeutic nucleic acid (TNA) and at least one pharmaceutically acceptable excipient, wherein the LNP comprises at least one lipid, and wherein the LNP is capable of delivering the TNA to a retinal cell.

2. The pharmaceutical composition of claim 1, wherein the LNP is capable of delivering the TNA to a photoreceptor (PR) cell.

3. The pharmaceutical composition of claim 1, wherein the LNP is capable of delivering the TNA to a retinal pigment epithelium (RPE) cell.

4. The pharmaceutical composition of any of claims 1-3, wherein the LNP is capable of delivering the TNA to a photoreceptor (PR) cell and a retinal pigment epithelium (RPE) cell, wherein expression of the TNA in the PR cell and expression of the TNA in RPE cell is evenly distributed.

5. The pharmaceutical composition of any one of claims 1-4, wherein the pharmaceutical composition is for administration to a subject.

6. The pharmaceutical composition of claim 5, wherein the pharmaceutical composition is for administration to a subject via subretinal injection, suprachoroidal injection, or intravitreal injection.

7. The pharmaceutical composition of claim 5, wherein the LNP/TNA is for administration at a dose of about 0.03 μg to about 2.0 μg.

8. The pharmaceutical composition of claim 5, wherein the LNP/TNA is for administration at a dose of about 0.1 μg to about 1.0 μg.

9. The pharmaceutical composition of claim 5, wherein the LNP/TNA is for administration at a dose of about 0.1 μg to about 0.5 μg.

10. The pharmaceutical composition of claim 5, wherein the subject is a human in need of treatment with LNP encapsulated with TNA.

11. The pharmaceutical composition of any one of claims 1-10, wherein the LNP is capable of being internalized into the PR cell and/or the RPE cell.

12. The pharmaceutical composition of any of claims 1-11, wherein retinal degeneration does not exceed more than 10% of outer nuclear layer (ONL) loss after 7 days.

13. The pharmaceutical composition of any of claims 1-12, wherein retinal degeneration does not exceed more than 10% of outer nuclear layer (ONL) loss after 14 days.

14. The pharmaceutical composition of any of claims 1-13, wherein retinal degeneration does not exceed more than 10% of outer nuclear layer (ONL) loss after 21 days.

15. The pharmaceutical composition of any of claims 1-14, wherein retinal degeneration does not exceed more than 10% of outer nuclear layer (ONL) loss after 4 weeks.

16. The pharmaceutical composition of any one of claims 13-15, wherein ONL loss is measured by Optical Coherence Tomography (OCT).

17. The pharmaceutical composition of any one of claims 1-16, wherein the LNP comprises a lipid selected from the group consisting of: a cationic lipid, a sterol or a derivative thereof, a non-cationic lipid, and a PEGylated lipid.

18. The pharmaceutical composition of any one of claims 1-17, wherein the TNA is encapsulated in the lipid.

19. The pharmaceutical composition of any one of claims 1-18, wherein the TNA is selected from the group consisting of minigenes, plasmids, minicircles, small interfering RNA (siRNA), microRNA (miRNA), antisense oligonucleotides (ASO), ribozymes, closed-ended (ceDNA), ministring, doggybone™, protelomere closed ended DNA, or dumbbell linear DNA, dicer-substrate dsRNA, small hairpin RNA (shRNA), asymmetrical interfering RNA (aiRNA), microRNA (miRNA), mRNA, tRNA, rRNA, gRNA, DNA viral vectors, viral RNA vector, non-viral vector and any combination thereof.

20. The pharmaceutical composition of claim 19, wherein the TNA is ceDNA.

21. The pharmaceutical composition of claim 19, wherein the ceDNA is linear duplex DNA.

22. The pharmaceutical composition of claim 19, wherein the TNA is mRNA.

23. The pharmaceutical composition of claim 19, wherein the TNA is siRNA.

24. The pharmaceutical composition of claim 19, wherein the TNA is a plasmid.

25. The pharmaceutical composition of any one of claims 1-24, wherein the pharmaceutical composition is administered to a subject.

26. The pharmaceutical composition of claim 25, wherein the subject is a human patient in need of treatment with the TNA encapsulated by the LNP.

27. The pharmaceutical composition of claim 17, wherein the cationic lipid is represented by Formula (I): 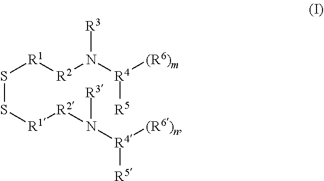
or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or -[C(R^{a})₂]₂CR^{a};
R^{a}, for each occurrence, is independently H or C₁₋₃ alkyl;
or alternatively, when R⁴ is —C(R^{a})₂CR^{a}, or -[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or -[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

28. The pharmaceutical composition of claim 17, wherein the cationic lipid is represented by Formula (II): 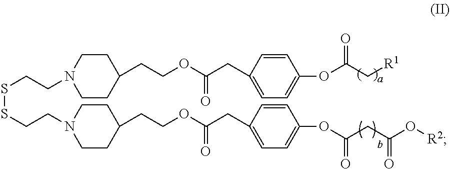
or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

29. The pharmaceutical composition of claim 28, wherein the cationic lipid is 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(tridecan-5-yl) nonanedioate (Lipid 58), represented by the following structural formula:

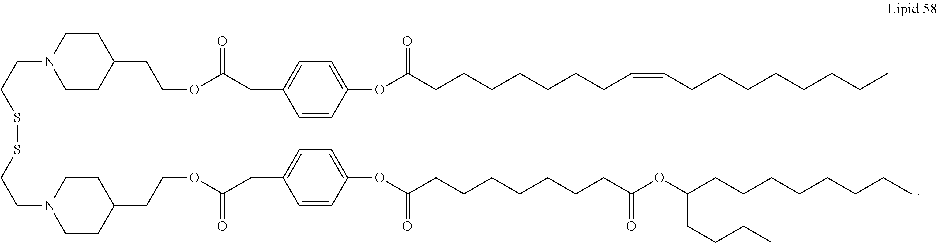 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

30. The pharmaceutical composition of claim 17, wherein the lipid is represented by the Formula (V): 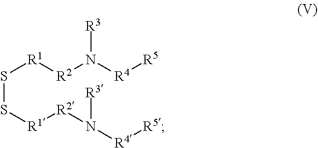
or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R^{b} are each halo or cyano.

31. The pharmaceutical composition of claim 17, wherein the cationic lipid is represented by Formula (XV): 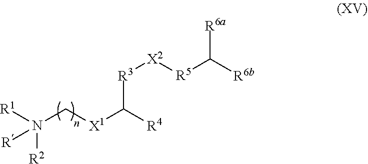
or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is protonated;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₆ unbranched alkyl, C₂-C₁₆ unbranched alkenyl, or 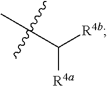
wherein:
R^{4a} and R^{4b} are each independently C₁-C₁₆ unbranched alkyl or C₂-C₁₆ unbranched alkenyl;
R⁵ is absent, C₁-C₅ alkylene, or C₂-C₅ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6.

32. The pharmaceutical composition of claim 17, wherein the cationic lipid is represented by Formula (XX): 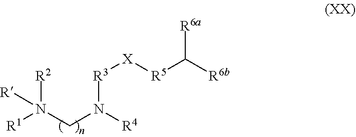
or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is protonated;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or 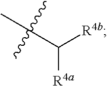
wherein:
R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6.

33. The pharmaceutical composition of claim 17, wherein the cationic lipid is selected from any lipid in Table 2, Table 5, Table 6, Table 7, or Table 8.

34. The pharmaceutical composition of claim 17, wherein the cationic lipid is Lipid A represented by the following structure: 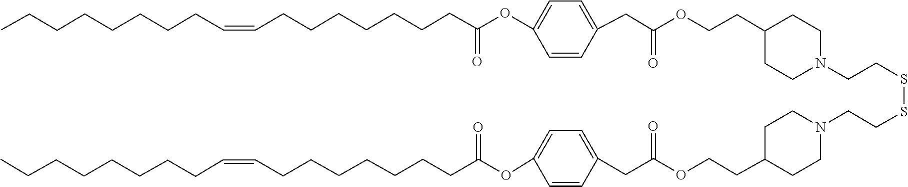
or a pharmaceutically acceptable salt thereof.

35. The pharmaceutical composition of claim 17, wherein the cationic lipid is 1-(4-(2-(2-(1-(2-((2-(4-(2-(2-(4-(oleoyloxy)phenyl)acetoxy)ethyl)piperidin-1-yl)ethyl)disulfaneyl)ethyl)piperidin-4-yl)ethoxy)-2-oxoethyl)phenyl) 9-(tridecan-5-yl) nonanedioate (Lipid 58), represented by the following structural formula: 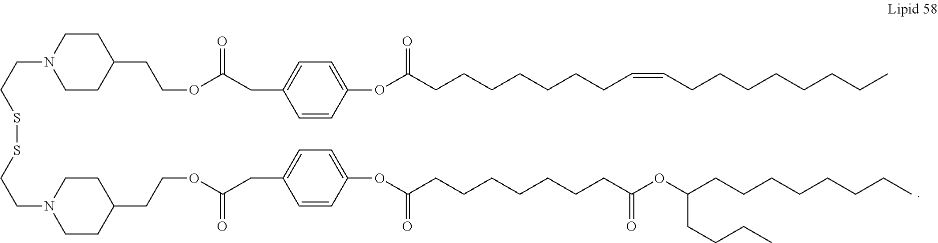 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]
or a pharmaceutically acceptable salt thereof.

36. The pharmaceutical composition of claim 17, wherein the sterol or a derivative thereof is a cholesterol.

37. The pharmaceutical composition of claim 17, wherein the sterol or a derivative thereof is beta-sitosterol.

38. The pharmaceutical composition of claim 17, wherein the non-cationic lipid is selected from the group consisting of distearoyl-sn-glycero-phosphoethanolamine (DSPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, and mixtures thereof.

39. The pharmaceutical composition of claim 38, wherein the non-cationic lipid is selected from the group consisting of dioleoylphosphatidylcholine (DOPC), distearoylphosphatidylcholine (DSPC), and dioleoyl-phosphatidylethanolamine (DOPE).

40. The pharmaceutical composition of claim 17, wherein the PEGylated lipid is selected from the group consisting of PEG-dilauryloxypropyl; PEG-dimyristyloxypropyl; PEG-dipalmityloxypropyl, PEG-distearyloxypropyl; l-(monomethoxy-polyethyleneglycol)-2,3-dimyristoylglycerol (DMG-PEG); 1,2-distearoyl-sn-glycero-3-phosphoethanolamine-N-[azido(polyethylene glycol], and distearoyl-rac-glycerol-poly(ethylene glycol) (DSG-PEG); PEG-dilaurylglycerol; PEG-dipalmitoylglycerol; PEG-disterylglycerol; PEG-dilaurylglycamide; PEG-dimyristylglycamide; PEG-dipalmitoylglycamide; PEG-disterylglycamide; (1-[8′-(Cholest-5-en-3[beta]-oxy)carboxamido-3′,6′-dioxaoctanyl]carbamoyl-[omega]-methyl-poly(ethylene glycol) (PEG-cholesterol); 3,4-ditetradecoxylbenzyl-[omega]-methyl-poly(ethylene glycol) ether (PEG-DMB), and 1,2-dimyristoyl-sn-glycero-3-phosphoethanolamine-N-[methoxy(polyethylene glycol) (DSPE-PEG), and 1,2-distearoyl-sn-glycero-3-phosphoethanolamine-N-poly(ethylene glycol)-hydroxyl (DSPE-PEG-OH).

41. The pharmaceutical composition of claim 40, wherein the PEGylated lipid is DMG-PEG, DSPE-PEG, DSPE-PEG-OH, DSG-PEG, or a combination thereof.

42. The pharmaceutical composition of claim 40 or 41, wherein the PEGylated lipid is DMG-PEG2000, DSPE-PEG2000, DSPE-PEG2000—OH, DSG-PEG2000, or a combination thereof.

43. The pharmaceutical composition of any one of claims 27-35, wherein the cationic lipid is present at a molar percentage of about 30% to about 80%.

44. The pharmaceutical composition of any one of claims 36 or 37, wherein the sterol is present at a molar percentage of about 20% to about 50%.

45. The pharmaceutical composition of any one of claims 38 or 39, wherein the non-cationic lipid is present at a molar percentage of about 2% to about 20%.

46. The pharmaceutical composition of any one of claims 40-42, wherein the PEGylated lipid is present at a molar percentage of about 2.1% to about 10% or wherein the PEGylated lipid is present at a molar percentage of about 1% to about 2%.

47. The pharmaceutical composition of any one of claims 1-46, further comprising dexamethasone palmitate.

48. The pharmaceutical composition of claim 34, wherein the LNP comprises Lipid A, DOPC, cholesterol and DMG-PEG.

49. The pharmaceutical composition of claim 34, wherein the LNP comprises Lipid A, DOPC, cholesterol, DMG-PEG, and DSPE-PEG.

50. The pharmaceutical composition of claim 34, wherein the LNP comprises Lipid A, DOPE, cholesterol and DMG-PEG.

51. The pharmaceutical composition of claim 34, wherein the LNP comprises Lipid A, DOPE, cholesterol, DMG-PEG, and DSPE-PEG.

52. The pharmaceutical composition of claim 34, wherein the LNP comprises Lipid A, DSPC, cholesterol and DMG-PEG.

53. The pharmaceutical composition of claim 34, wherein the LNP comprises Lipid A, DSPC, cholesterol, DMG-PEG, and DSPE-PEG.

54. The pharmaceutical composition of claim 34, wherein the LNP comprises Lipid A, DOPC, beta-sitosterol and DMG-PEG.

55. The pharmaceutical composition of claim 34, wherein the LNP comprises Lipid A, DOPC, beta-sitosterol, DMG-PEG, and DSPE-PEG.

56. The pharmaceutical composition of claim 34, wherein the LNP comprises Lipid A, DOPE, beta-sitosterol and DMG-PEG.

57. The pharmaceutical composition of claim 34, wherein the LNP comprises Lipid A, DOPE, beta-sitosterol, DMG-PEG, and DSPE-PEG.

58. The pharmaceutical composition of claim 34, wherein the LNP comprises Lipid A, DSPC, beta-sitosterol and DMG-PEG.

59. The pharmaceutical composition of claim 34, wherein the LNP comprises Lipid A, DSPC, beta-sitosterol, DMG-PEG, and DSPE-PEG.

60. The pharmaceutical composition of any one of claims 48-59, wherein the DMG-PEG is DMG-PEG2000.

61. The pharmaceutical composition of any one of claims 48-60, wherein the DSPE-PEG is DSPE-PEG2000.

62. The pharmaceutical composition of any one of claims 49, 51, 53, 55, 57 or 59, wherein the DSPE-PEG is DSPE-PEG5000.

63. The pharmaceutical composition of claim 62, wherein the LNP comprises Lipid A, DOPC, sterol, DMG-PEG and DSPE-PEG at molar ratios of about 51: 7.3: 38.3: 2.9: 0.5.

64. The pharmaceutical composition of any one of claims 1 to 63, wherein the LNP has a total lipid to TNA ratio of about 10:1 to about 40:1.

65. A method of treating an ocular disorder in a subject, the method comprising administering to the subject a therapeutically effective amount of the pharmaceutical composition of any one of claims 1 to 64.

66. The method according to claim 65, wherein the subject is a human.

67. The method according to claim 65 or 66, wherein the ocular disorder is Stargardt macular dystrophy.

68. The method according to claim 65 or 66, wherein the ocular disorder is LCA10.

69. The method according to claim 65 or 66, wherein the ocular disorder is Usher syndrome.

70. The method according to claim 65 or 66, wherein the ocular disorder is wet AMD.

71. A method of treating a genetic disorder in a subject, comprising administering to the subject an effective amount of the pharmaceutical composition of any one of claims 1-64.

72. The method according to claim 71, wherein the subject is a human.

73. The method according to claim 72, wherein the genetic disorder is an ocular disorder.

74. A method of delivering a therapeutic nucleic acid (TNA) or increasing the concentration of the TNA in the retina of a subject, the method comprising administering to the subject a therapeutically effective amount of the pharmaceutical composition of any one of claims 1-64.
